(19) United States
(12) Patent Application Publication
(10) Pub. No.: US 2025/0290083 A1
(43) Pub. Date: Sep. 18, 2025

(54) USE OF MORPHOGENIC FACTORS FOR THE IMPROVEMENT OF GENE EDITING

(71) Applicant: PIONEER HI-BRED INTERNATIONAL, INC., JOHNSTON, IA (US)

(72) Inventors: STEPHEN LAWRENCE GASIOR, JOHNSTON, IA (US); WILLIAM JAMES GORDON-KAMM, URBANDALE, IA (US); KEITH S LOWE, JOHNSTON, IA (US); DAVID J PETERSON, AMES, IA (US); SERGEI SVITASHEV, JOHNSTON, IA (US); JOSHUA K YOUNG, JOHNSTON, IA (US)

(73) Assignee: PIONEER HI-BRED INTERNATIONAL, INC., JOHNSTON, IA (US)

(21) Appl. No.: 19/220,291

(22) Filed: May 28, 2025

Related U.S. Application Data
(63) Continuation of application No. 17/717,357, filed on Apr. 11, 2022, now Pat. No. 12,344,851.
Application No. 17/717,357 is a continuation of application No. 16/979,917, filed on Sep. 11, 2020, now Pat. No. 11,332,752.
(60) Provisional application No. 62/641,725, filed on Mar. 12, 2018.
(60) Provisional application No. 62/641,733, filed on Mar. 12, 2018.

(51) Int. Cl. C12N 15/82

(52) U.S. Cl. CPC C12N 15/8213; C12N 15/8205; C12N 15/8207; C12N 15/8216

(57) ABSTRACT

Methods and compositions are provided for the improvement of double-strand-break-inducing agent activity in eukaryotic cells, through the usage of one or more morphogenic factors or developmental genes. The morphogenic factor may be provided to the same cell or to a different cell than that comprising or receiving the double-strand-break-inducing agent. The morphogenic factor may be provided to a cell as a polynucleotide composition on a recombinant vector, and may be placed on the vector outside of a T-DNA border. The morphogenic factor may be provided via an upregulation of an endogenous gene. The morphogenic factor, or the double-strand-break-inducing agent, may further comprise a cell penetrating peptide. The morphogenic factor may be co-introduced with a vector comprising RepA.

### CROSS-REFERENCE TO RELATED APPLICATIONS

This application is s a continuation of U.S. application Ser. No. 17/717,357, now allowed, which claims priority a continuation of U.S. application Ser. No. 16/979,917, allowed as U.S. patent Ser. No. 11/332,752, which claims priority to a 371 National Phase Entry of PCT/US19/21631 filed on 11 Mar. 2019, which claims the benefit of U.S. Provisional Patent Application Ser. No. 62/641,733 filed 12 Mar. 2018 and of U.S. Provisional Patent Application Ser. No. 62/641,725 filed 12 Mar. 2018, each of which are herein incorporated by reference in their entireties.

### REFERENCE TO SEQUENCE LISTING SUBMITTED ELECTRONICALLY

The official copy of the sequence listing is submitted electronically via EFS-Web as an .XML formatted sequence listing with a file named “106602-US-CON-2_Application.DOCX” and have a size of 144,985 bytes and is filed concurrently with the specification. The sequence listing comprised in this ASCII formatted document is part of the specification and is herein incorporated by reference in its entirety.

### FIELD OF THE INVENTION

The present disclosure relates generally to the field of plant molecular biology, specifically the targeted modification of polynucleotides in plants.

### BACKGROUND

Standard methods for genome modification of an organism have multiple steps that may limit speed and efficiency, and can negatively impact product development timelines. In plants, there are growing season limitations that can further delay the creation and testing of plants comprising modified polynucleotides. For example, many standard methods of transformation and regeneration require the use of high auxin or cytokinin levels and require steps involving either embryogenic callus formation or organogenesis, leading to procedures that take many weeks before producing plants for growth in a greenhouse setting following transformation.

Currently, plant genetic transformation and genome modification protocols require delivery of various DNA vectors coding for different components, including for example double-strand break reagents (Cas9 nuclease and guide RNA (gRNA)), a selectable marker, morphogenic factors (for example, ODP2, BBM, and WUS) and donor DNA (in case of a polynucleotide insertion or a swap).

Multiple, co-delivered DNA molecules tend to co-integrate into a DSB site through a non-homologous end joining (NHEJ) repair pathway, significantly reducing the efficiency of homology directed repair (HDR)-based genome editing. Modifying protocols for the delivery of some components, such as a morphogenic factor, and minimizing delivery of DNA may be beneficial and lead to higher frequencies of quality HDR events.

There remains a need for a faster, more efficient system to produce organisms comprising desired polynucleotide modifications quickly and efficiently.

### SUMMARY OF INVENTION

In some aspects, methods and compositions are provided for editing a polynucleotide in the genome of a cell, by introduction of a double-strand-break-inducing agent and a morphogenic factor.

In some aspects, the morphogenic factor is provided to the same cell as the cell receiving, or comprising, the double-strand-break-inducing agent. In some aspects, the morphogenic factor is provided to a different cell than the cell receiving, or comprising, the double-strand-break inducing agent. In some aspects, the morphogenic factor is provided on the same construct as that comprising a double-strand-break-inducing agent. In some aspects, the morphogenic factor is provided on a different construct as that comprising a double-strand break-inducing agent. In some aspects, a construct is provided that comprises a heterologous polynucleotide for introduction into a target cell, wherein said construct may optionally further comprise either a morphogenic factor, a double-strand-break-inducing agent, or both.

In some aspects, the morphogenic factor is provided on the same recombinant DNA vector as a heterologous polynucleotide, wherein the morphogenic factor is located outside of the TDNA borders of the vector.

In some aspects, the morphogenic factor is endogenous to the cell receiving, or comprising, the double-strand-break-inducing agent, and expression or activity of the endogenous morphogenic factor is altered. In some aspects, the alteration is up-regulation. In some aspects, the alteration is down-regulation.

In some aspects, a cell penetrating peptide is linked to the morphogenic factor, enabling its movement between cells.

In some aspects, the morphogenic factor is co-introduced with RepA.

In some aspects, combinations of other aspects are utilized to improve polynucleotide modification. For example, in one non-limiting embodiment, a cell penetrating peptide is linked to a morphogenic factor that is provided to an adjacent cell to the one receiving, or comprising, the double-strand-break-inducing agent. For example, in one non-limiting embodiment, an endogenous morphogenic factor is upregulated in a cell adjacent to the cell receiving, or comprising, the double-strand-break-inducing agent. Any combination of any aspect described herein may be utilized to improve polynucleotide modification by a double-strand-break-inducing agent provided to a cell.

In some aspects, the morphogenic factor is provided as a polynucleotide sequence encoding a polypeptide. In some aspects, the morphogenic factor shares at least 80%, at least 85%, at least 90%, at least 95%, at least 96%, at least 97%, at least 98%, at least 99%, at least 99.5%, or greater than 99.5% identity with a sequence selected from the group consisting of: SEQID NOs: 6-10, 17-21, and 48-73. In some aspects, the morphogenic factor shares at least 80%, at least 85%, at least 90%, at least 95%, at least 96%, at least 97%, at least 98%, at least 99%, at least 99.5%, or greater than 99.5% identity with a sequence selected from the group consisting of: SEQID NOs: 1-5, 11-16, 22, and 23-47. In some aspects, the morphogenic factor is selected from the group consisting of: Wuschel, Ovule Development Protein, and Babyboom. In some aspects the morphogenic factor is selected from the group consisting of: WUS1, WUS2, WUS3, WOX2A, WOX4, WOX5, WOX9, BBM2, BMN2, BMN3, and ODP2.

In some aspects, the morphogenic factor is operably linked to a heterologous promoter.

In any aspect, the editing of a polynucleotide may be selected from the group consisting of: insertion of at least one polynucleotide, deletion of at least one polynucleotide, modification of at least one polynucleotide, substitution of at least one polynucleotide, and a combination of at least two of the preceding.

In some aspects, the double-strand-break-inducing agent comprises a Cas endonuclease, a TALEN, a zinc finger endonuclease, a meganuclease, or a restriction endonuclease.

In some aspects, the Cas endonuclease has one or more mutations, that eliminate double-strand break activity and/or single-strand nicking activity.

In some aspects, the double-strand-break-inducing agent is provided as a ribonucleoprotein complex comprising a Cas endonuclease protein and a guide RNA.

In some aspects, the method or composition may further comprise a heterologous polynucleotide donor DNA molecule introduced into said target cell.

In some aspects, the modification of a polynucleotide confers a benefit to an organism comprising, or derived from, said target cell. In some aspects, said benefit is selected from the group consisting of: improved health, improved growth, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction.

In some aspects, the cell is a eukaryotic cell. In some aspects, the cell is a plant cell. In some aspects, the plant cell. In some aspects, the plant cell is obtained from or derived from a monocot or dicot plant. In some aspects, the plant cell is obtained from or derived from a monocot selected from the group consisting of: Zea mays, Sorghum bicolor, Sorghum vulgare, Triticum aestivum, Medicago sativa, Oryza sativa, Setaria italica, and Saccharum spp. In some aspects, the plant cell is obtained from or derived from a dicot selected from the group consisting of: Helianthus annuus, Glycine max, Nicotiana tabacum, Gossypium barbadense, Gossypium hirsutum, Manihot esculenta, Beta vulgaris, Brassica spp., and Arabidposis thaliana.

In some aspects, editing of a polynucleotide in the genome of a plant cell via any of the compositions or methods described herein results in the modulation of a trait of agronomic importance in a plant, selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition; as compared to an isoline plant not comprising or derived from a cell whose genome was edited with said double-strand-break-inducing agent.

In some aspects, methods and compositions are provided for editing a polynucleotide in the genome of a cell, by introduction of a double-strand-break-inducing agent and a morphogenic factor, wherein said cell does not have the morphogenic factor stably integrated into its genome.

In some aspects, methods and compositions are provided for editing a polynucleotide in the genome of a cell, by introduction of a double-strand-break-inducing agent and a morphogenic factor, wherein the morphogenic factor is co-introduced with REPA.

In some aspects, methods and compositions are provided for editing a polynucleotide in the genome of a cell, by introduction of a double-strand-break-inducing agent and a morphogenic factor, wherein the morphogenic factor is co-introduced with a polynucleotide encoding deactivated Cas9 molecule fused to a repressor, wherein said polynucleotide is located outside of the T-DNA borders.

### BRIEF DESCRIPTION OF THE DRAWINGS AND THE SEQUENCE LISTING

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

FIG. 1A depicts a DNA vector encoding for WUS (polynucleotide SEQID NO: 23 encoding polypeptide SEQID NO:48) operably linked to a PLTP promoter (SEQID NO:74).

FIG. 1B depicts a maize-optimized S. pyogenes Cas9 protein (polynucleotide SEQID NOs: 103-104 encoding polypeptide SEQID NOs: 108-109) and an appropriate gRNA (SEQID NO: 82).

FIG. 1C depicts a DNA vector comprising a selectable marker gene (NPTII, polynucleotide SEQID NO: 77 encoding polypeptide SEQID NO:100) flanked with homology arms (SEQID NOs: 78 and 79) as donor DNA.

FIG. 2A depicts the general location of the designed guides (UTRs and exon/intron structures based on the maize B73 version of the gene, using genomic maize Variety 1 contig sequences.

FIG. 2B depicts the map of a dCas9-GRA-CBF1A vector.

FIG. 2C shows transformation enhancement in embryos bombarded with the sense guide RNAs.

FIG. 2D shows plants after 5-7 weeks of outgrowth.

FIG. 2E shows the protein sequence differences between the WUS2 from a recombinant vector (polynucleotide SEQID NOs: 73 (exon 1) and 24 (exon 2) encoding polypeptide SEQID NO:49), from a native B73 maize plant (polynucleotide SEQID NO:25 encoding polypeptide SEQID NO:50), and from a native Variety 1 maize plant (polynucleotide SEQID NO:26 encoding polypeptide SEQID NO:51). The consensus sequence is given as SEQID NO:106.

FIG. 3 depicts an example of a recombinant vector comprising morphogenic factor polynucleotides outside of the left border.

Table 1 depicts some of the compositions useful for the methods described herein, given as SEQID NOs: 1-110.

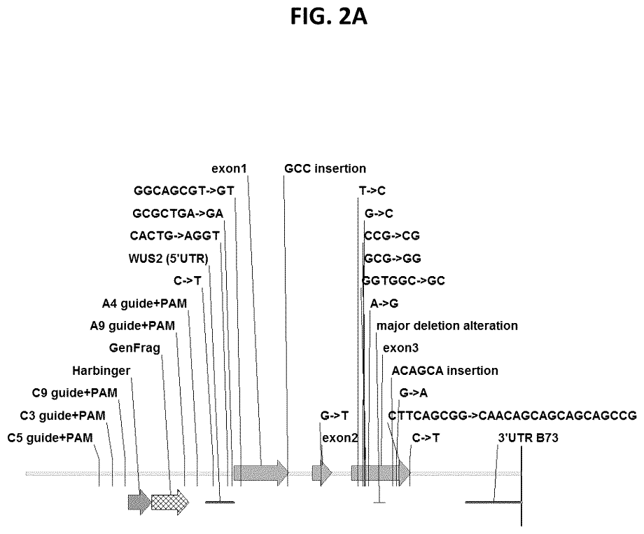

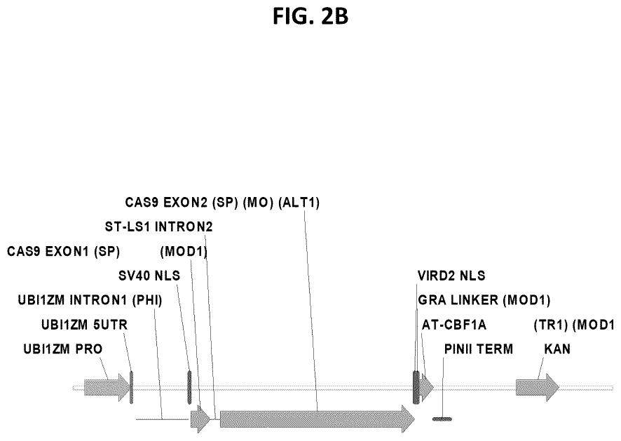

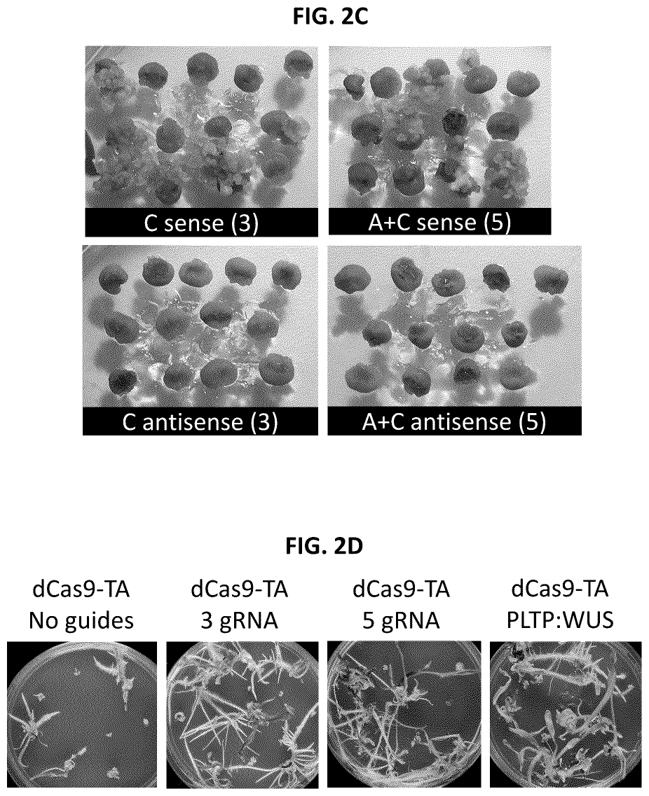

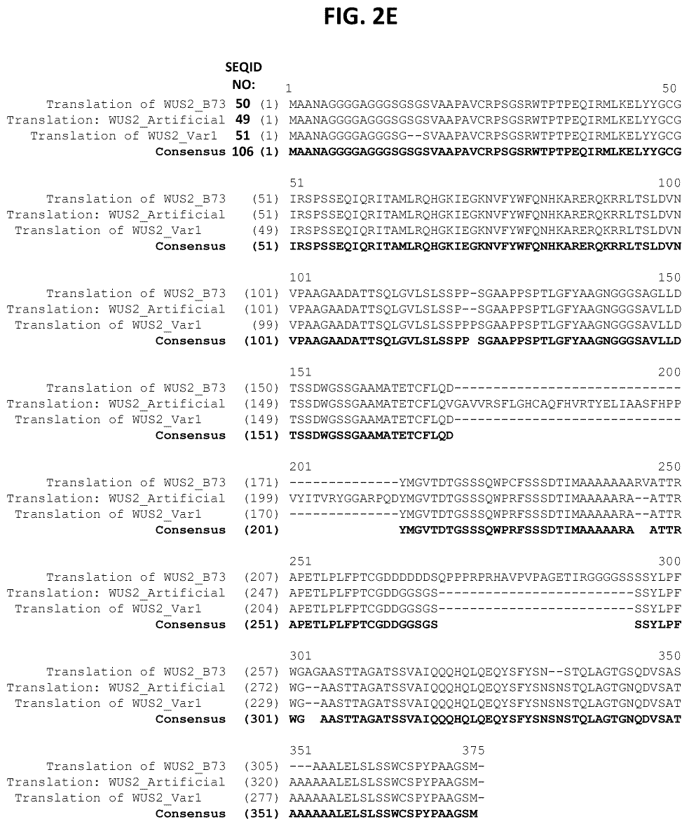

TABLE 1
Sequence Descriptions

| SEQID | | | | |
|---|---|---|---|---|
| NO | Description | Type | Genus | Species |
| 1 | Z. mays BBM2 coding sequence | DNA | Zea | mays |
| 2 | Oryza sativa BBM1 coding sequence | DNA | Oryza | sativa |
| 3 | Oryza sativa BBM2 coding sequence | DNA | Oryza | sativa |
| 4 | Oryza sativa BBM3 coding sequence | DNA | Oryza | sativa |
| 5 | Sorghum bicolor BBM2 coding sequence | DNA | Sorghum | bicolor |
| 6 | Z. mays BBM2 protein sequence | PRT | Zea | mays |
| 7 | Oryza sativa BBM1 protein sequence | PRT | Oryza | sativa |
| 8 | Oryza sativa BBM2 protein sequence | PRT | Oryza | sativa |
| 9 | Oryza sativa BBM3 protein sequence | PRT | Oryza | sativa |
| 10 | Sorghum bicolor BBM2 protein sequence | PRT | Sorghum | bicolor |
| 11 | Z. mays ODP2 coding sequence | DNA | Zea | mays |
| 12 | Z. mays ODP2 coding sequence (synthetic) | DNA | Zea | mays |
| 13 | Sorghum bicolor ODP2 coding sequence | DNA | Sorghum | bicolor |
| 14 | Setaria italica ODP2 coding sequence | DNA | Setaria | italica |
| 15 | Brachypodium distachyum ODP2 coding sequence | DNA | Brachypodium | distachyum |
| 16 | Sorghum bicolor ODP2 genomic sequence | DNA | Sorghum | bicolor |
| 17 | ODP2 (ALT3) | PRT | Zea | mays |
| 18 | Z. mays ODP2 protein sequence | PRT | Zea | mays |
| 19 | Sorghum bicolor ODP2 protein sequence | PRT | Sorghum | bicolor |
| 20 | Setaria italica ODP2 protein sequence | PRT | Setaria | italica |
| 21 | Brachypodium distachyum ODP2 protein | PRT | Brachypodium | distachyum |
| | sequence | | | |
| 22 | odp2 (ALT3) | DNA | Zea | mays |
| 23 | wus | DNA | Zea | mays |
| 24 | wus2 artificial vector exon 2 | DNA | Zea | mays |
| 25 | wus2 B73 | DNA | Zea | mays |
| 26 | wus2 Variety 1 | DNA | Zea | mays |
| 27 | wus2 (ALT1) | DNA | Zea | mays |
| 28 | Arabidopsis thaliana WUS coding sequence | DNA | Arabidopsis | thaliana |
| 29 | Lotus japonicus WUS coding sequence | DNA | Lotus | japonicus |
| 30 | Glycine max WUS coding sequence | DNA | Glycine | max |
| 31 | Camelina sativa WUS coding sequence | DNA | Camelina | sativa |
| 32 | Capsella rubella WUS coding sequence | DNA | Copsella | rubella |
| 33 | Arabis alpina WUS coding sequence | DNA | Arabis | alpina |
| 34 | Raphanus sativus WUS coding sequence | DNA | Raphanus | sativus |
| 35 | Brassica napus WUS coding sequence | DNA | Brassica | napus |
| 36 | Brassica oleracea var. oleracea WUS coding | DNA | Brassica | oleracea |
| | sequence | | | |
| 37 | Helianthus annuus WUS coding sequence | DNA | Helianthus | annuus |
| 38 | Populus trichocarpa WUS coding sequence | DNA | Populus | trichocarpa |
| 39 | Vitus vinifera WUS coding sequence | DNA | Vitus | vinifera |
| 40 | Arabidopsis thaliana WUS coding sequence (soy | DNA | Arabidopsis | thaliana |
| | optimized) | | | |
| 41 | Lotus japonicus WUS coding sequence (soy | DNA | Lotus | japonicus |
| | optimized) | | | |
| 42 | Medicago trunculata WUS coding sequence (soy | DNA | Medicago | trunculata |
| | optimized) | | | |
| 43 | Petunia hybrida WUS coding sequence (soy | DNA | Petunia | hybrida |
| | optimized) | | | |
| 44 | Phaseolus vulgaris WUS coding sequence (soy | DNA | Phaseolus | vulgaris |
| | optimized) | | | |
| 45 | 3-ZM-WUS1-Z. mays WUS1 coding sequence | DNA | Zea | mays |
| 46 | 5-ZM-WUS2-Z. mays WUS2 coding sequence | DNA | Zea | mays |
| 47 | 7-ZM-WUS3-Z. mays WUS3 coding sequence | DNA | Zea | mays |
| 48 | WUS | PRT | Zea | mays |
| 49 | WUS artificial vector | PRT | Zea | mays |
| 50 | WUS2 B73 | PRT | Zea | mays |
| 51 | WUS2 Variety 1 | PRT | Zea | mays |
| 52 | WUS2 (ALT1) | PRT | Zea | mays |
| 53 | Arabidopsis thaliana WUS protein sequence | PRT | Arabidopsis | thaliana |
| 54 | Lotus japonicus WUS protein sequence | PRT | Lotus | japonicus |
| 55 | Glycine max WUS protein sequence | PRT | Glycine | max |
| 56 | Camelina sativa WUS protein sequence | PRT | Camelina | saliva |
| 57 | Capsella rubella WUS protein sequence | PRT | Copsella | rubella |
| 58 | Arabis alpina WUS protein sequence | PRT | Arabis | alpina |
| 59 | Raphanus sativus WUS protein sequence | PRT | Raphanus | sativus |
| 60 | Brassica napus WUS protein sequence | PRT | Brassica | napus |
| 61 | Brassica oleracea var. oleracea WUS protein | PRT | Brassica | oleracea |
| | sequence | | | |
| 62 | Helianthus annuus WUS protein sequence | PRT | Helianthus | annuus |
| 63 | Populus trichocarpa WUS protein sequence | PRT | Populus | trichocarpa |
| 64 | Vitus vinifera WUS protein sequence | PRT | Vitus | vinifera |
| 65 | Arabidopsis thaliana WUS protein sequence | PRT | Arabidopsis | thaliana |
| 66 | Lotus japonicus WUS protein sequence | PRT | Lotus | japonicus |
| 67 | Medicago trunculata WUS protein sequence | PRT | Medicago | trunculata |
| 68 | Petunia hybrida WUS protein sequence | PRT | Petunia | hybrida |
| 69 | Phaseolus vulgaris WUS protein sequence | PRT | Phaseolus | vulgaris |
| 70 | 4-ZM-WUS1-Z. mays WUS1 protein sequence | PRT | Zea | mays |
| 71 | 6-ZM-WUS2-Z. mays WUS2 protein sequence | PRT | Zea | mays |
| 72 | 8-ZM-WUS3-Z. mays WUS3 protein sequence | PRT | Zea | mays |
| 73 | wus2 artificial vector exon 1 | DNA | Zea | mays |
| 74 | PLTP promoter | DNA | Zea | mays |
| 75 | UBI promoter | DNA | Zea | mays |
| 76 | potato LS1 intron 2 | DNA | Solanum | tuberosum |
| 77 | donor DNA GOI (NPTII selectable marker) | DNA | Escherichia | coli |
| 78 | donor DNA homology arm 1 | DNA | Artificial | |
| 79 | donor DNA homology arm 2 | DNA | Artificial | |
| 80 | gRNA (gene deletion site 1) | DNA | Artificial | |
| 81 | gRNA (gene deletion site 2) | DNA | Artificial | |
| 82 | gRNA (Example 3 site specific gene insertion) | DNA | Artificial | |
| 83 | At-cbf1a | DNA | Arabidopsis | thaliana |
| 84 | polIII promoter | DNA | Zea | mays |
| 85 | gRNA | DNA | Artificial | |
| 86 | gRNA C5 (Example 4) | DNA | Artificial | |
| 87 | gRNA C3 (Example 4) | DNA | Artificial | |
| 88 | gRNA C9 (Example 4) | DNA | Artificial | |
| 89 | gRNA A9 (Example 4) | DNA | Artificial | |
| 90 | gRNA A4 (Example 4) | DNA | Artificial | |
| 91 | PLTP promoter | DNA | Zea | mays |
| 92 | PLTP 5′UTR | DNA | Zea | mays |
| 93 | UBI3 promoter | DNA | Setarica | italica |
| 94 | UBI3 intron 1 | DNA | Setarica | italica |
| 95 | PINII terminator | DNA | Solanum | tuberosum |
| 96 | ALS promoter | DNA | Sorghum | bicolor |
| 97 | ALS (HRA) | DNA | Zea | mays |
| 98 | PEPC1 terminator | DNA | Sorghum | bicolor |
| 99 | T28 terminator | DNA | Oryza | sativa |
| 100 | NPTII selectable marker | PRT | Escherichia | coli |
| 101 | At-CBF1A | PRT | Arabidopsis | thaliana |
| 102 | cas9 (with ST-L1S intron, SV40 NLS, VIRD2 | DNA | Streptococcus | pyogenes |
| | NLS) | | | |
| 103 | cas9 exon 1 | DNA | Streptococcus | pyogenes |
| 104 | cas9 exon 2 | DNA | Streptococcus | pyogenes |
| 105 | cas9 (D10A + H840A) | DNA | Streptococcus | pyogenes |
| 106 | consensus sequence for WUS (n = any or no | PRT | Artificial | |
| | aamino cid) - FIG. 2E | | | |
| 107 | Cas9 | PRT | Streptococcus | pyogenes |
| 108 | Cas9 exon 1 | PRT | Streptococcus | pyogenes |
| 109 | Cas9 exon 2 | PRT | Streptococcus | pyogenes |
| 110 | dCas9 (D10A + H840A) | PRT | Streptococcus | pyogenes |

### DETAILED DESCRIPTION

Various compositions and methods for modifying a polynucleotide target site in a cell, for example a plant cell, are provided. The modification can include an insertion, deletion, mutation, replacement, or molecular alteration of a nucleotide sequence. The target site is modified through the activity of a double-strand break-inducing agent that recognizes a recognition sequence within the target site.

Double-strand breaks induced by double-strand inducing agents can result in the induction of DNA repair mechanisms, including the non-homologous end-joining pathway, and homologous recombination. Error-prone DNA repair mechanisms can produce mutations at double-strand break sites. The nonhomologous end-joining (NHEJ) pathways are the most common repair mechanism that serve to bring the broken polynucleotide ends together (Bleuyard et al. (2006) DNA Repair 5:1-12). The structural integrity of chromosomes is typically preserved by the repair, but deletions, insertions, or other rearrangements are possible. The two ends of one double-strand break are the most prevalent substrates of NHEJ (Kirik et al. (2000) EMBO J 19:5562-6). If two different double-strand breaks occur, however, the free ends from different breaks can be ligated to one another, resulting in chromosomal deletions (Siebert and Puchta (2002) Plant Cell 14:1121-31), or chromosomal translocations between different chromosomes (Pacher et al. (2007) Genetics 175:21-9).

Episomal DNA molecules, for example T-DNAs, can also be ligated into the double-strand break, resulting in integration of the episomal DNA molecule into the host genome (Chilton and Que (2003) Plant Physiol 133:956-65; Salomon and Puchta (1998) EMBO J 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (S, G2, M phases of a cell cycle) (Molinier et al. (2004) Plant Cell 16:342-52). Heterologous, ectopic and/or epigenic DNA sequences may also serve as a DNA repair template for homologous recombination (Puchta (1999) Genetics 152:1173-81).

DNA double-strand breaks (DSBs) appear to be an effective factor to stimulate homologous recombination pathways in every organism tested to date (Puchta et al. (1995) Plant Mol Biol 28:281-92; Tzfira and White (2005) Trends Biotechnol 23:567-9; Puchta (2005) J Exp Bot 56:1-14). For example, using DNA break-inducing a, a two- to nine-fold increase of homologous recombination was observed between artificially constructed homologous DNA repeats in plants (Puchta et al. (1995) Plant Mol Biol 28:281-92). Thus, double-strand break-inducing agents can be used for targeted modification of polynucleotides in organisms and the provision of one or more morphogenic proteins enhances the frequency of targeted modification.

Morphogenic proteins can enhance the rate of targeted modification of a target site in a cell of an organism, such as a plant, that has been induced by a double-strand break-inducing agent. In some methods, at least one morphogenic protein and a double-strand break-inducing agent are introduced into a cell having a target site with at least one recognition sequence. The double-strand break-inducing agent recognizes the recognition sequence and introduces a double-strand break at or near the recognition sequence to produce a modified target site. Modifications to the target site can include a deletion, mutation, replacement, chemical or molecular modification, homologous recombination, or insertion of a nucleotide sequence. In certain embodiments, the target site is stably integrated into the genome of the plant. In some of these embodiments, the genomic target site is a native genomic target site. These methods can be used to stimulate recombination at a target site, integrate polynucleotides into a target site, invert or excise a polynucleotide, directly select transformed organisms, minimize or eliminate expression resulting from random integration into the genome of an organism, combine multiple transfer cassettes, silence genes, and characterize transcriptional regulatory regions.

The methods disclosed herein involve the use of a morphogenic factor, such as babyboom (BBM)/ovule development protein (ODP) and/or Wuschel (WUS), that serves to enhance and promote the polynucleotide modification effected by the double-strand-break-inducing agent. The morphogenic factor may be provided to a cell as a protein or as a polynucleotide encoding a protein. In one aspect, a heterologous morphogenic factor is provided to the target cell. In one aspect, an endogenous morphogenic factor in the target cell or adjacent cell is activated or upregulated. In one aspect, a morphogenic factor is provided to an adjacent cell instead of to the target cell, whereby the target cell benefits from the presence of the morphogenic factor in the adjacent cell. In one aspect, the morphogenic factor is provided to the target cell as part of a transformation cassette, with the polynucleotide encoding the morphogenic factor located outside of the transformation borders such that its integration into the target cell genome is transient.

Here we describe different approaches for cellular genome editing to leverage the benefits of morphogenic factors, without stably integrating the morphogenic factor into the target cell that receives the double-strand-break agent components for targeted polynucleotide modification. Such approaches include: leveraging the expression of an endogenous morphogenic factor in the target cell or adjacent cell, delivery of a morphogenic factor to a different cell than the target cell, and/or providing the morphogenic factor to the target cell as part of a recombinant vector wherein said morphogenic factor lies outside of the T-DNA borders.

These novel methods provide improved efficiency of genome editing, improved percentage of regenerated transformed plants, lower attrition rate of the target cells/organisms, and reduced integration of unwanted DNA.

### Definitions

Terms used in the claims and specification are defined as set forth below unless otherwise specified. It must be noted that, as used in the specification and the appended claims, the singular forms “a,” “an” and “the” include plural referents unless the context clearly dictates otherwise.

The term “genome” refers to the entire complement of genetic material (genes and non-coding sequences) that is present in each cell of an organism, or virus or organelle; and/or a complete set of chromosomes inherited as a (haploid) unit from one parent. The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

The terms “provided (to)” and “introduced (into)” are used interchangeably herein. In another aspect, it is meant that a particular composition becomes functionally associated with a cell or other molecule. In one aspect, it is meant that a particular composition is taken up by the cell into its interior.

By the term “endogenous” it is meant a sequence or other molecule that naturally occurs in a cell or organism. In one aspect, an endogenous polynucleotide is normally found in the genome of a cell; that is, not heterologous.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more compositions, such as those provided herein, may be entirely synthetic.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The relationship between two or more polynucleotides or polypeptides may be determined. Polynucleotide and polypeptide sequences, fragments thereof, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment.

Sequence relationships may be defined by their composition comparisons, or by their ability to hybridize, or by their ability to engage in homologous recombination.

Methods of alignment of sequences for comparison are well known in the art. Thus, the determination of percent sequence identity between any two sequences can be accomplished using a mathematical algorithm. Sequence alignments and percent identity or similarity calculations may be determined using a variety of comparison methods designed to detect homologous sequences including, but not limited to, the MegAlign program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

“Sequence identity” or “identity” in the context of nucleic acid or polypeptide sequences refers to the nucleic acid bases or amino acid residues in two sequences that are the same when aligned for maximum correspondence over a specified comparison window. The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program. Unless otherwise stated, sequence identity/similarity values provided herein refer to the value obtained using GAP Version 10 (GCG, Accelrys, San Diego, CA) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, an “isolated” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture media components when produced by recombinant techniques, or substantially free of chemical precursors or other molecules when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide of the invention or biologically active portion thereof is recombinantly produced, optimally culture medium represents less than about 30%, 20%, 10%, 5%, or 1% (by dry weight) of chemical precursors or non-protein-of-interest molecules.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of another organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene, is recombinant.

The terms “recombinant polynucleotide”, “recombinant nucleotide”, “recombinant DNA” and “recombinant DNA construct” are used interchangeably herein. A recombinant construct comprises an artificial or heterologous combination of nucleic acid sequences, e.g., regulatory and coding sequences that are not found together in nature. For example, a transfer cassette can comprise restriction sites and a heterologous polynucleotide of interest. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments provided herein. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

“Open reading frame” is abbreviated ORF.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. As used herein, the term “morphogenic factor” means a morphogenic gene and/or the protein expressed by a morphogenic gene.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225 236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated yet has an effect on cellular processes. The terms “complement” and “reverse complement” are used interchangeably herein with respect to mRNA transcripts, and are meant to define the antisense RNA of the message.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

The term “expression”, as used herein, refers to the production of a functional end-product (e.g., an mRNA, guide RNA, or a protein) in either precursor or mature form.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

The term “fragment” refers to a contiguous set of polynucleotides or polypeptides. In one embodiment, a fragment is 2, 3, 4, 5, 6, 7 8, 9, 10, 11, 12, 13, 14, 15, 16, 17, 18, 19, 20, or greater than 20 contiguous polynucleotides. In one embodiment, a fragment is 2, 3, 4, 5, 6, 7 8, 9, 10, 11, 12, 13, 14, 15, 16, 17, 18, 19, 20, or greater than 20 contiguous polypeptides. A fragment may or may not exhibit the function of a sequence sharing some percent identity over the length of said fragment.

The terms “fragment that is functionally equivalent”, “functional fragment”, and “functionally equivalent fragment” are used interchangeably herein. These terms refer to a portion or subsequence of a nucleic acid fragment or polypeptide that displays the same activity or function as the longer sequence from which it derives. In one example, the fragment retains the ability to alter gene expression, create a double strand nick or break, or produce a certain phenotype whether or not the fragment encodes the whole protein as found in nature. In some aspects, part of the activity is retained. In some aspects, all of the activity is retained.

The terms “variant that is functionally equivalent”, “functional variant”, and “functionally equivalent variant” are used interchangeably herein. These terms refer to a nucleic acid fragment or polypeptide that displays the same activity or function as the source sequence from which it derives, but differs from the source sequence by at least one nucleotide or amino acid. In one example, the variant retains the ability to alter gene expression, create a double strand nick or break, or produce a certain phenotype. In some aspects, part of the activity is retained. In some aspects, all of the activity is retained.

A functional fragment or functional variant shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native source polynucleotide or polypeptide, and retains at least partial activity.

“Modified”, “edited”, or “altered, with respect to a polynucleotide or target sequence, refers to a nucleotide sequence that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) association of another molecule or atom via covalent, ionic, or hydrogen bonding, or (v) any combination of (i)-(iv).

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides may be but are not limited to intracellular localization signals.

An “optimized” polynucleotide is a sequence that has been optimized for improved expression in a particular heterologous host cell.

A “codon-modified gene” or “codon-preferred gene” or “codon-optimized gene” is a gene having its frequency of codon usage designed to mimic the frequency of preferred codon usage of the host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

A “polynucleotide of interest” includes any nucleotide sequence encoding a protein or polypeptide that improves desirability of an organism, for example, animals or plants. Polynucleotides of interest: include, but are not limited to, polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

As used herein, a “genomic region of interest” is a segment of a chromosome in the genome of a plant that is desirable for introducing a double-strand break, a polynucleotide of interest, or a trait of interest. The genomic region of interest can include, for example, one or more polynucleotides of interest. Generally, a genomic region of interest of the present invention comprises a segment of chromosome that is 0-15 centimorgan (cM).

The terms “knock-out”, “gene knock-out” and “genetic knock-out” are used interchangeably herein. A knock-out represents a DNA sequence of a cell that has been rendered partially or completely inoperative by targeting with a DSB agent; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a DSB agent (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

“Introducing” is intended to mean presenting to a target, such as a cell or organism, a polynucleotide or polypeptide or polynucleotide-protein complex, in such a manner that the component(s) gains access to the interior of a cell of the organism or to the cell itself.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

As used herein, the terms “target site”, “target sequence”, and “target polynucleotide” are used interchangeably herein and refer to a polynucleotide sequence in the genome of a plant cell or yeast cell that comprises a recognition site for a double-strand-break-inducing agent.

A “target cell” is a cell that comprises a target sequence and is the object for receipt of a particular double-strand-break-inducing agent.

A “break-inducing agent” is a composition that creates a cleavage in at least one strand of a polynucleotide. In some aspect, a break-inducing agent may be capable of, or have its activity altered such that it is capable of, creating a break in only one strand of a polynucleotide. Producing a single-strand-break in a double-stranded target sequence may be referred to herein as “nicking” the target sequence.

The term “double-strand-break-inducing agent”, or equivalently “double-strand-break agent” or “DSB agent”, as used herein refers to any composition which produces a double-strand break in a target polynucleotide sequence; that is, creates a break in both strands of a double stranded polynucleotide. Examples of a DSB agent include, but are not limited to: meganucleases, TAL effector nucleases, Argonautes, Zinc Finger nucleases, and Cas endonucleases (either individually or as part of a ribonucleoprotein complex). Producing the double-strand break in a target sequence may be referred to herein as “cutting” or “cleaving” the target sequence. In some aspects, the DSB agent is a nuclease. In some aspects, the DSB agent is an endonuclease. An “endonuclease” refers to an enzyme that cleaves the phosphodiester bond within a polynucleotide chain. In some embodiments, the double-strand break results in a “blunt” end of a double-stranded polynucleotide, wherein both strands are cut directly across from each other with no nucleotide overhang generated. A “blunt” end cut of a double-stranded polynucleotide is created when a first cleavage of the first stand polynucleotide backbone occurs between a first set of two nucleotides on one strand, and a second cleavage of the second strand polynucleotide backbone occurs between a second set of two nucleotides on the opposite strand, wherein each of the two nucleotides of the first set are hydrogen bonded to one of the two nucleotides of the second set, resulting in cut strands with no nucleotide on the cleaved end that is not hydrogen bonded to another nucleotide on the opposite strand. In some embodiments, the double-strand break results in a “sticky” end of a double-stranded polynucleotide, wherein cuts are made between nucleotides of dissimilar relative positions on each of the two strands, resulting in a polynucleotide overhang of one strand compared to the other. A “sticky” end cut of a double-stranded polynucleotide is created when a first cleavage of the first strand polynucleotide backbone occurs between a first set of two nucleotides on one strand, and a second cleavage of the second strand polynucleotide backbone occurs between a second set of two nucleotides on the opposite strand, wherein no more than one nucleotide of the first set is hydrogen bonded to one of the nucleotides of the second set on the opposite strand, resulting in an “overhang” of at least one polynucleotide on one of the two strands wherein the lengths of the two resulting cut strands are not identical. In some embodiments, the DSB agent comprises more than one type of molecule. In one non-limiting example, the DSB agent comprises an endonuclease protein and a polynucleotide, for example a Cas endonuclease and a guide RNA. In some aspects, the DSB agent is a fusion protein comprising a plurality of polypeptides. In one non-limiting example, the DSB agent is a Cas endonuclease with a deactivated nuclease domain, and another polypeptide with nuclease activity.

As used herein, the term “recognition site” refers to a polynucleotide sequence to which a double-strand-break-inducing agent is capable of alignment, and may optionally contact, bind, and/or effect a double-strand break. The terms “recognition site” and “recognition sequence” are used interchangeably herein. The recognition site can be an endogenous site in a host (such as a yeast, animal, or plant) genome, or alternatively, the recognition site can be heterologous to the host (yeast, animal, or plant) and thereby not be naturally occurring in the genome, or the recognition site can be found in a heterologous genomic location compared to where it occurs in nature. The length and the composition of a recognition site can be characteristic of, and may be specific to, a particular double-strand-break-inducing agent. The cleavage site of a DSB agent may be the same or different than the recognition site, and may be the same or different than the binding site.

As used herein, the term “endogenous recognition (or binding or cleavage) site” refers to a double-strand-break-inducing agent recognition (or binding or cleavage) site that is endogenous or native to the genome of a host (such as a plant, animal, or yeast) and is located at the endogenous or native position of that recognition (or binding or cleavage) site in the genome of the host (such as a plant, animal, or yeast). The length of the recognition (or binding or cleavage) site can vary, and includes, for example, recognition (or binding or cleavage) sites that are at least 1, 2, 3, 4, 5, 6, 7, 8, 9, 10, 11, 12, 13, 14, 15, 16, 17. 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more than 70 nucleotides in length. The composition of the recognition (or binding or cleavage) site can vary, and includes, for example, a plurality of specific nucleotides whose compositions are recognized by the DSB agent. In some aspects, the plurality of specific nucleotides is contiguous in the primary sequence. In some aspects, the plurality of specific nucleotides is non-contiguous in the primary sequence. It is further possible that the recognition site could be palindromic, that is, the sequence on one strand reads the same in the opposite direction on the complementary strand. The binding and/or nick/cleavage site could be within the recognition sequence or the binding and/or nick/cleavage site could be outside of the recognition sequence. In another variation, the DSB cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

As used herein, the term “target recognition site” refers to the polynucleotide sequence to which a double-strand-break-inducing agent is capable of aligning perfectly (i.e., zero nucleotide mismatches, gaps, or insertions), and in some aspects, induces a double-strand break.

As used herein, the term “target binding site” refers to the polynucleotide sequence at which the double-strand-break-inducing agent is capable of forming a functional association, and to which it forms bonds with complementary nucleotides of the target polynucleotide strand, with perfect alignment (i.e., zero nucleotide mismatches, gaps, or insertions).

As used herein, the term “target cleavage site” refers to the polynucleotide sequence at which a double-strand-break-inducing agent is capable of producing a double-strand break, with perfect alignment (i.e., zero nucleotide mismatches, gaps, or insertions).

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes but is not limited to: the novel Cas-delta protein disclosed herein, a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. The Cas-delta endonucleases of the disclosure may include those having RuvC or RuvC-like nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity.

The terms “cascade” and “cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of an organism, produced via sexual or asexual reproduction.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%), at least about 400% or more higher than the control.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kb” means kilobase(s).

### Compositions for Modifying the Genome of a Target Cell

### Morphogenic Factors

The present disclosure comprises methods and compositions for producing genomic modifications in an organism using a double-strand-break agent and a morphogenic factor. Morphogenic factors can enhance the rate, efficiency, and efficacy of targeted polynucleotide modification by a number of mechanisms, some of which are related to the capability of stimulating growth of a cell or tissue, including but not limited to promoting progression through the cell cycle, inhibiting cell death, such as apoptosis, stimulating cell division, and/or stimulating embryogenesis. The polynucleotides can fall into several categories, including but not limited to, cell cycle stimulatory polynucleotides, developmental polynucleotides, anti-apoptosis polynucleotides, hormone polynucleotides, transcription factors, or silencing constructs targeted against cell cycle repressors or pro-apoptotic factors. Methods and compositions for rapid and efficient transformation of plants by transforming cells of plant explants with an expression construct comprising a heterologous nucleotide encoding a morphogenic factor are described in US Patent Application Publication No. US2017/0121722 (published 4 May 2017).

A morphogenic factor (gene or protein) may involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof.

In some aspects, the morphogenic factor is a molecule selected from one or more of the following categories: 1) cell cycle stimulatory polynucleotides including plant viral replicase genes such as RepA, cyclins, E2F, prolifera, cdc2 and cdc25; 2) developmental polynucleotides such as Lec1, Kn1 family, WUSCHEL, Zwille, BBM, Aintegumenta (ANT), FUS3, and members of the Knotted family, such as Kn1, STM, OSH1, and SbH1; 3) anti-apoptosis polynucleotides such as CED9, Bcl2, Bcl-X(L), Bcl-W, A1, McL-1, Mac1, Boo, and Bax-inhibitors; 4) hormone polynucleotides such as IPT, TZS, and CKI-1; and 5) silencing constructs targeted against cell cycle repressors, such as Rb, CKl, prohibitin, and wee1, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference, cosuppression, chimerplasty, or transposon insertion.

In some aspects, the morphogenic gene is a member of the WUS/WOX gene family (WUS1, WUS2, WUS3, WOX2A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39. The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Modulation of WUS/WOX is expected to modulate plant and/or plant tissue phenotype including plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Expression of Arabidopsis WUS can induce stem cells in vegetative tissues, which can differentiate into somatic embryos (Zuo, et al. (2002) Plant J 30:349-359). Also of interest in this regard would be a MYB 118 gene (see U.S. Pat. No. 7,148,402), MYB 115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

In some embodiments, the morphogenic gene or protein is a member of the AP2/ERF family of proteins. The AP2/ERF family of proteins is a plant-specific class of putative transcription factors that regulate a wide variety of developmental processes and are characterized by the presence of an AP2 DNA binding domain that is predicted to form an amphipathic alpha helix that binds DNA (PFAM Accession PF00847). The AP2 domain was first identified in APETALA2, an Arabidopsis protein that regulates meristem identity, floral organ specification, seed coat development, and floral homeotic gene expression. The AP2/ERF proteins have been subdivided into distinct subfamilies based on the presence of conserved domains. Initially, the family was divided into two subfamilies based on the number of DNA binding domains, with the ERF subfamily having one DNA binding domain, and the AP2 subfamily having 2 DNA binding domains. As more sequences were identified, the family was subsequently subdivided into five subfamilies: AP2, DREB, ERF, RAV, and others. (Sakuma et al. (2002) Biochem Biophys Res Comm 290:998-1009).

Members of the APETALA2 (AP2) family of proteins function in a variety of biological events, including but not limited to, development, plant regeneration, cell division, embryogenesis, and morphogenic (see, e.g., Riechmann and Meyerowitz (1998) Biol Chem 379:633-646; Saleh and Pages (2003) Genetika 35:37-50 and Database of Arabidopsis Transcription Factors at daft.cbi.pku.edu.cn). The AP2 family includes, but is not limited to, AP2, ANT, Glossy15, AtBBM, BnBBM, and maize ODP2/BBM.

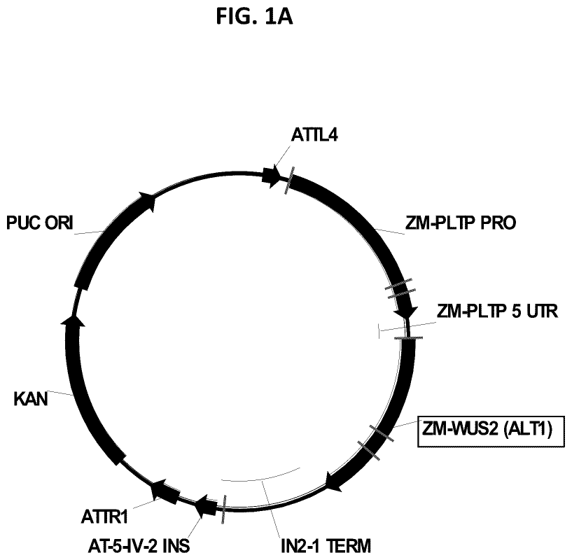

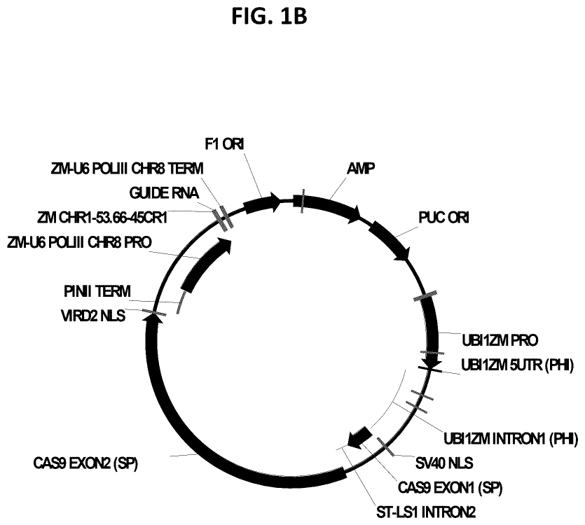

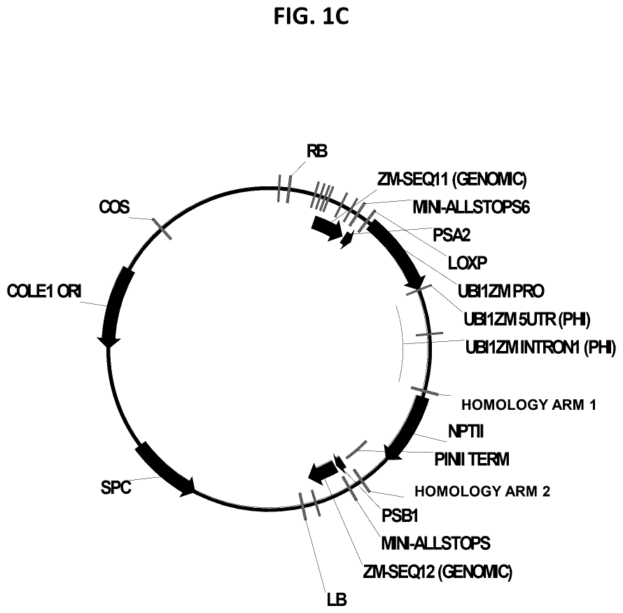

Other morphogenic genes useful in the present disclosure include, but are not limited to, Ovule Development Protein 2 (ODP2) polypeptides, and related polypeptides, e.g., Babyboom (BBM) protein family proteins. In an aspect, the polypeptide comprising the two AP2-DNA binding domains is an ODP2, BBM2, BMN2, or BMN3 polypeptide. The ODP2 polypeptides of the disclosure contain two predicted APETALA2 (AP2) domains and are members of the AP2 protein family (PFAM Accession PF00847). The AP2 family of putative transcription factors has been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins. The ODP2 polypeptides share homology with several polypeptides within the AP2 family, e.g., see FIG. 1 of U.S. Pat. No. 8,420,893, which is incorporated herein by reference in its entirety, provides an alignment of the maize and rice ODP2 polypeptides with eight other proteins having two AP2 domains. A consensus sequence of all proteins appearing in the alignment of U.S. Pat. No. 8,420,893 is also provided in FIG. 1 therein.

In some embodiments, the morphogenic factor is a babyboom (BBM) polypeptide, which is a member of the AP2 family of transcription factors. The BBM protein from Arabidopsis (AtBBM) is preferentially expressed in the developing embryo and seeds and has been shown to play a central role in regulating embryo-specific pathways. Overexpression of AtBBM has been shown to induce spontaneous formation of somatic embryos and cotyledon-like structures on seedlings. See, Boutiler et al. (2002) The Plant Cell 14:1737-1749. The maize BBM protein also induces embryogenesis and promotes transformation (See, U.S. Pat. No. 7,579,529, which is herein incorporated by reference in its entirety). Thus, BBM polypeptides stimulate proliferation, induce embryogenesis, enhance the regenerative capacity of a plant, enhance transformation, and as demonstrated herein, enhance rates of targeted polynucleotide modification. As used herein “regeneration” refers to a morphogenic response that results in the production of new tissues, organs, embryos, whole plants or parts of whole plants that are derived from a single cell or a group of cells. Regeneration may proceed indirectly via a callus phase or directly, without an intervening callus phase. “Regenerative capacity” refers to the ability of a plant cell to undergo regeneration.

Other morphogenic genes useful in the present disclosure include, but are not limited to, LEC1 (Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabidopsis AGL15 gene (Harding et al., 2003, Plant Physiol. 133:653-663), and the FUSCA gene (Castle and Meinke, Plant Cell 6:25-41), and the PICKLE gene (Ogas et al., 1999, PNAS 96:13839-13844).

The morphogenic factor can be derived from a monocot. In various aspects, the morphogenic factor is derived from barley, maize, millet, oats, rice, rye, Setaria sp., sorghum, sugarcane, switchgrass, triticale, turfgrass, or wheat.

The morphogenic factor can be derived from a dicot. The morphogenic factor can be derived from kale, cauliflower, broccoli, mustard plant, cabbage, pea, clover, alfalfa, broad bean, tomato, cassava, soybean, canola, alfalfa, sunflower, safflower, tobacco, Arabidopsis, or cotton.

The present disclosure encompasses isolated or substantially purified polynucleotide or polypeptide morphogenic factor compositions.

The morphogenic factor may be altered in various ways including amino acid substitutions, deletions, truncations, and insertions. Methods for such manipulations are generally known in the art. For example, amino acid sequence variants of the morphogenic proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, polynucleotides or polypeptides having homology to a known morphogenic factor and/or sharing conserved functional domains can be identified by screening sequence databases using programs such as BLAST, or using standard nucleic acid hybridization techniques known in the art, for example as described in Tijssen (1993) Laboratory Techniques in Biochemistry and Molecular Biology—Hybridization with Nucleic Acid Probes, Part I, Chapter 2 (Elsevier, NY); Ausubel et al., eds. (1995) Current Protocols in Molecular Biology, Chapter 2 (Greene Publishing and Wiley-Interscience, NY); and, Sambrook et al. (1989) Molecular Cloning: A Laboratory Manual (2d ed., Cold Spring Harbor Laboratory Press, Plainview, NY).

In some aspects, the morphogenic gene is selected from the group consisting of: SEQID NOs:1-5, 11-16, 22, and 23-47. In some aspects, the morphogenic protein is selected from the group consisting of: SEQID NOs: 6-10, 17-21, and 48-73.

In some aspects, a plurality of morphogenic factors is selected. When multiple morphogenic factors are used, the polynucleotides encoding each of the factors can be present on the same expression cassette or on separate expression cassettes. Likewise, the polynucleotide(s) encoding the morphogenic factor(s) and the polynucleotide encoding the double-strand break-inducing agent can be located on the same or different expression cassettes. When two or more factors are coded for by separate expression cassettes, the expression cassettes can be provided to the organism simultaneously or sequentially.

In some aspects, the expression of the morphogenic factor is transient. In some aspects, the expression of the morphogenic factor is constitutive. In some aspects, the expression of the morphogenic factor is specific to a particular tissue or cell type. In some aspects, the expression of the morphogenic factor is temporally regulated. In some aspects, the expression of the morphogenic factor is regulated by an environmental condition, such as temperature, time of day, or other factor. In some aspects, the expression of the morphogenic factor is stable. In some aspects, expression of the morphogenic factor is controlled. The controlled expression may be a pulsed expression of the morphogenic factor for a particular period of time. Alternatively, the morphogenic factor may be expressed in only some transformed cells and not expressed in others. The control of expression of the morphogenic factor can be achieved by a variety of methods as disclosed herein.

### Double Strand Break Agents

A double-strand-break-inducing agent (equivalently, “double-strand-break agent” or “DSB agent”) described herein may be introduced to a target polynucleotide to create a “functional association”. A “functional association” means that the DSB agent is introduced to a target polynucleotide molecule, may optionally bind to it, and is capable of producing a double-strand-break on the backbone of the target polynucleotide to which it is introduced. The position (location with respect to the polynucleotide sequence) and nature (blunt-end, sticky-end, or mixed) of the double-strand break is dependent upon the exact DSB agent used.

The term “double-strand-break-inducing agent” as used herein refers to any composition which produces a double-strand break in a target sequence in the genome of an organism. Double-strand-break-inducing agents may be proteins that include but are not limited to: restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, DC)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

An “engineered double-strand-break-inducing agent” refers to any double-strand-break-inducing agent that is engineered (modified or derived) from its native form to specifically recognize and induce a double-strand break in the desired recognition site. Thus, an engineered double-strand-break-inducing agent can be derived from a native, naturally-occurring nuclease or it could be artificially created or synthesized. The modification of the nuclease can be as little as one nucleotide. In some embodiments, the engineered double-strand-break-inducing agent induces a double-strand break in a recognition site, wherein the recognition site was not a sequence that would have been recognized by a native (non-engineered or non-modified) double-strand-break-inducing agent. Producing a double-strand break in a recognition site or other DNA can be referred to herein as “cutting” or “cleaving” the recognition site or other DNA.

In some aspects, the DSB agent can be provided as a polypeptide, that can be purified and substantially free of other molecules, or can be in association with one or more heterologous components. In one embodiment, the DSB agent is a polypeptide and a polynucleotide. In one embodiment, the DSB agent is a fusion protein, comprising two or more domains, wherein one domain can effect the cleavage of a target polynucleotide. In one embodiment, the DSB agent is a plurality of polypeptides.

In some aspects, the DSB agent can be provided via a polynucleotide encoding the DSB agent polypeptide. Such a polynucleotide encoding may optionally be modified to substitute codons having a higher frequency of usage in a particular host cell or organism, as compared to the naturally-occurring polynucleotide sequence. For example, the polynucleotide encoding the DSB agent can be modified to substitute codons having a higher frequency of usage in a maize or soybean plant, as compared to the naturally occurring polynucleotide sequence.

### Restriction Endonucleases

Endonucleases include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex.

Type I and Type III restriction endonucleases recognize specific recognition sites, but typically cleave at a variable position from the recognition site, which can be hundreds of base pairs away from the recognition site. In Type II systems the restriction activity is independent of any methylase activity, and cleavage typically occurs at specific sites within or near to the recognition site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, DC)).

### Meganucleases

A “meganuclease” refers to a homing endonuclease, which like restriction endonucleases, bind and cut at a specific recognition site, however the recognition sites for meganucleases are typically longer, about 18 bp or more. In some embodiments of the invention, the meganuclease has been engineered (or modified) to cut a specific endogenous recognition sequence, wherein the endogenous target sequence prior to being cut by the engineered double-strand-break-inducing agent was not a sequence that would have been recognized by a native (non-engineered or non-modified) endonuclease.

A “meganuclease polypeptide” refers to a polypeptide having meganuclease activity and thus capable of producing a double-strand break in the recognition sequence.

Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing open reading frames, introns, and inteins, respectively. For example, intron-, intein-, and freestanding gene encoded meganuclease from Saccharomyces cerevisiae are denoted I-SceI, PI-SceI, and F-SceII, respectively. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Examples of meganucleases include, but are not limited to: I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any functional variants or fragments thereof.

### Argonautes

Small non-coding RNAs are one type of contributor to gene regulation, and require a unique class of proteins called Argonautes. Argonaute proteins are highly specialized binding modules that can bind small non-coding RNAs and control protein synthesis, affect messenger RNA stability and even participate in the production of a new class of small RNAs, Piwi-interacting RNAs. Argonautes coordinate downstream gene-silencing events by interacting with other protein factors.

First identified in plants and subsequently discovered to be ubiquitous in many organisms, Argonaute proteins are defined by the presence of PAZ (Piwi-Argonaute-Zwille) and PIWI domains. They are evolutionarily conserved and can be phylogenetically subdivided into the Ago subfamily and the Piwi subfamily. Ago proteins are ubiquitously expressed and bind to siRNAs or miRNAs to guide post-transcriptional gene silencing either by destabilization of the mRNA or by translational repression. The expression of Piwi proteins is mostly restricted to the germ line and Piwi proteins associate with piRNAs to facilitate silencing of mobile genetic elements.

Many Argonaute proteins bind RNA guides to cleave foreign RNA, while others are capable of cleaving plasmid and genomic DNA. Natronobacterium gregoryi Argonaute uses 5′ phosphorylated DNA guides (rather than the RNA guides employed by Cas9 or Cpf1), without requiring a PAM sequence, to randomly remove 1-20 nucleotides from the cleavage site specified by the gDNA.

### TAL Effector Nucleases (TALENs)

TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

### Zinc-Finger Endonucleases

Zinc fingers are structural domains found in eukaryotic proteins which control gene transcription. The zinc finger domain of the Cys₂His₂class of ZFPs is a polypeptide structural motif folded around a bound zinc ion, and has a sequence of the form X₃-Cys-X₂₋₄-Cys-X₁₂-His-X₃₋₅-His-X₄- (wherein X is any amino acid). The zinc finger is an independent folding domain which uses a zinc ion to stabilize the packing of an antiparallel j-sheet against an α-helix. There is a great deal of sequence variation in the amino acids designated as X, however, the two consensus histidine and cysteine residues are invariant. Although most ZFPs have a similar three-dimensional structure, they bind polynucleotides having a wide range of nucleotide sequences. the binding of the zinc finger domain is dependent on the sequence of the polynucleotides other than those which directly contact amino acids within the zinc finger domain.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms

Zinc-finger nucleases are site-specific endonucleases comprising two protein domains: a DNA-binding domain, comprising a plurality of individual zinc finger repeats that each recognize between 9 and 18 base pairs, and a DNA-cleavage domain that comprises a nuclease domain (typically Fok1). The cleavage domain dimerizes in order to cleave DNA; therefore, a pair of ZFNs are required to target non-palindromic target polynucleotides.

### Cas Endonucleases

As used herein, the term “Cas gene” refers to a gene that is generally coupled, associated or close to or in the vicinity of flanking CRISPR loci.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence.

Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position.

Cas endonucleases may be capable of forming a complex with a guide polynucleotide (e.g., guide RNA or gRNA) that is capable of recognizing, binding to, and optionally nicking, unwinding, or cleaving all or part of a target sequence. In some aspects, the guide polynucleotide/Cas endonuclease complex is capable of introducing a double-strand-break into a target polynucleotide. In some aspects, the guide polynucleotide comprises solely RNA, solely DNA, a chimeric molecule comprising both DNA and RNA, and/or comprises a chemically modified nucleotide. The guide polynucleotide (e.g., guide RNA) may be a single guide RNA (sgRNA) that is capable of binding to a sequence on the target polynucleotide.

Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases include, but are not limited to, Cas endonucleases identified from the following systems: Class 1, Class 2, Type I, Type II, Type III, Type IV, Type V, and Type VI. In some aspects, the Cas endonuclease is Cas3 (a feature of Class 1 type I systems), Cas9 (a feature of Class 2 type II systems) or Cas12 (Cpf1) (a feature of Class 2 type V systems).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10: 957-963).

A Cas endonuclease, effector protein, functional variant, or a functional fragment thereof, for use in the disclosed methods, can be isolated from a native source, or from a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

### Polynucleotides of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK: sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g., the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins (such as green fluorescent protein (GFP), cyan (CFP), yellow (YFP), red (RFP), and blue (BFP), (Shaner et al., 2005, Nature Methods 2:905-909)), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying agent, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Target Sites

The target site may be part of the organism's native genome or integrated therein or may be present on an episomal polynucleotide. The genomic target sequence may be on any region of any chromosome, and may or may not be in a region encoding a protein or RNA. The target site may be native to the cell or heterologous. In some embodiments, the heterologous target sequence may have been transgenically inserted into the organism's genome, and may be on any region of any chromosome, including an artificial or satellite chromosome, and may or may not be in a region encoding a protein or RNA. In some aspects, the target site polynucleotide is of nuclear origin (genomic), and may be either endogenous to the cell or may be heterologous (e.g. an introduced transgene). In some aspects, the target site polynucleotide is a plasmid or vector that exists within the cell or has been introduced. In some aspects, the target site polynucleotide exists in the cytoplasm of the cell (extra-nuclear). In some aspects, the target site polynucleotide exists in another organelle of the cell (e.g. plastid or mitochondrion).It is recognized that the cell or the organism may comprise multiple target sites, which may be located at one or multiple loci within or across chromosomes. Multiple independent manipulations of each target site in the organism can be performed using the presently disclosed methods.

The target site comprises at least one recognition sequence. The length of the recognition site sequence can vary, and includes, for example, sequences that are at least about 3, 4, 6, 8, 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 90, 100, or more nucleotides in length. In some embodiments, the recognition site is of a sufficient length to only be present in a genome of an organism one time. In some embodiments, the recognition site is palindromic, that is, the sequence on one strand reads the same in the opposite direction on the complementary strand. The double-strand break-inducing agent recognizes the recognition sequence and introduces a double-strand break at or near the recognition sequence. The nick/cleavage site could be within the sequence that is specifically recognized by the agent or the nick/cleavage site could be outside of the sequence that is specifically recognized by the agent. In some embodiments, the double-strand break is introduced about1,2,3,4,5,6,7,8,9, 10, 11, 12, 13, 14, 15, 16, 17, 18, 19,20,21,22,23,24, 25, 26, 27, 28, 29, 30, 40, 50, 60, 70, 80, 90, 100, 110, 120, 130, 140, 150, 160, 170, 180, 190, 200, or more nucleotides away from the recognition sequence.

In some embodiments, the cleavage occurs at nucleotide positions immediately opposite each other to produce a blunt end cut or, in alternative embodiments, the cuts are staggered to produce single-stranded overhangs, also called “sticky ends”, which can be either 5′ overhangs, or 3′ overhangs. The recognition sequence can be endogenous (native) or heterologous to the plant cell. When the recognition site is an endogenous sequence, it may be recognized by a naturally-occurring, or native double-strand break-inducing agent. Alternatively, an endogenous recognition sequence may be recognized and/or bound by a modified or engineered double-strand break-inducing agent designed or selected to specifically recognize the endogenous recognition sequence to produce a double-strand break.

### Expression Elements

Any polynucleotide encoding a Cas protein or other CRISPR system component disclosed herein may be functionally linked to a heterologous expression element, to facilitate transcription or regulation in a host cell. Such expression elements include but are not limited to: promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like.

Tissue-preferred promoters can be utilized to target enhanced expression within a particular plant tissue. Tissue-preferred promoters include, for example, WO2013103367 published 11 Jul. 2013, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J 4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8; Gotor et al., (1993) Plant J 3:509-18; Orozco et al., (1993) Plant Mol Biol 23:1129-38; Matsuoka et al., (1993) Proc. Natl. Acad. Sci. USA 90:9586-90; Simpson et al., (1958) EMBO J 4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersonii and Trema tomentosa); Leach and Aoyagi, (1991) Plant Sci 79:69-76 (A. rhizogenes rolC and rolD root-inducing genes); Teeri et al., (1989) EMBO J 8:343-50 (Agrobacterium wound-induced TR1′ and TR2′ genes); VfENOD-GRP3 gene promoter (Kuster et al., (1995) Plant Mol Biol 29:759-72); and rolB promoter (Capana et al., (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, CimI (cytokinin-induced message); cZ19B1 (maize 19 kDa zein); and milps (myo-inositol-1-phosphate synthase); and for example, those disclosed in WO2000011177 published 2 Mar. 2000 and U.S. Pat. No. 6,225,529. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO2000012733 published 9 Mar. 2000, where seed-preferred promoters from END1 and END2 genes are disclosed.

Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-II-27, WO1993001294 published 21 Jan. 1993), activated by hydrophobic electrophilic compounds used as pre-emergent herbicides, and the tobacco PR-1a promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Pathogen inducible promoters induced following infection by a pathogen include, but are not limited to those regulating expression of PR proteins, SAR proteins, beta-1,3-glucanase, chitinase, etc.

A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally-occurring stress conditions.

Another example of an inducible promoter useful in plant cells, is the ZmCAS1 promoter, described in US20130312137 published 21 Nov. 2013.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, NY: Academic Press), pp. 1-82.

Examples of promoters useful for the expression of morphogenic factors in cells include, but are not limited to: Figwort Mosaic Virus (FMV) promoter, FMV enhanced promoter, MMV enhanced promoter, Cauliflower Mosaic Virus (CaMV) promoter, CaMV enhanced promoter, 35S promoter, enhanced 35S promoter, minimal promoters, plant PLTP promoter, plant Ubiquitin promoter, plant Heat Shock Protein promoter, Nopaline Synthase (nos) promoter, plant Elongation Factor (EF) promoter, those described in U.S. Patent Application Publication No. 2017/0121722 (published 4 May 2017), those described in U.S. Pat. No. 8,710,206.

### Polynucleotide Constructs

One or more of the polynucleotide compositions herein may be provided to a cell as part of a expression construct, plasmid, or vector. Said construct may comprise one or more expression cassettes for transcription in the cell.

The transcriptional cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the potato proteinase inhibitor (PinII) gene or sequences from Ti-plasmid of A. tumefaciens, such as the nopaline synthase, octopine synthase and opaline synthase termination regions. See also, Guerineau et al., (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. 1989) Nucleic Acids Res. 17: 7891-7903; Joshi et al. (1987) Nucleic Acid Res. 15: 9627-9639.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′noncoding region) (Elroy-Stein, O., Fuerst, T. R., and Moss, B. (1989) PNAS USA, 86: 6126-6130); potyvirus leaders, for example, TEV leader (Tobacco Etch Virus) (Allison et al. (1986); MDMV leader (Maize Dwarf Mosaic Virus); Virology, 154: 9-20), and human immunoglobulin heavy-chain binding protein (BiP), (Macejak, D. G., and P. Sarnow (1991) Nature, 353: 90-94; untranslated leader from the coat protein MARNA of alfalfa mosaic virus (AMV RNA 4), (Jobling, S. A., and Gehrke, L., (1987) Nature, 325: 622-625; tobacco mosaic virus leader (TMV), (Gallie et al. (1989) Molecular Biology of RNA, pages 237-256, Gallie et al. (1987) Nucl. Acids Res. 15: 3257-3273; maize chlorotic mottle virus leader (MCMV) (Lornmel, S. A. et al. (1991) Virology, 81: 382-385). See also, Della-Cioppa et al. (1987) Plant Physiology, 84: 965-968; and endogenous maize 5′ untranslated sequences. Other methods known to enhance translation can also be utilized, for example, introns, and the like.

The expression cassettes may contain one or more than one gene or nucleic acid sequence to be transferred and expressed in the transformed plant. Thus, each nucleic acid sequence will be operably linked to 5′ and 3′ regulatory sequences. Alternatively, multiple expression cassettes may be provided.

### Modification of a Target Polynucleotide in a Cell

According to the presently disclosed methods, morphogenic factors are used to enhance the modification of a target site polynucleotide within a cell that is effected by DSB agent activity. In some aspects, the morphogenic factor and the DSB agent may be introduced into the same cell. In some aspects, the morphogenic factor and the DSB agent may each be introduced into a different cell. In some aspects, the morphogenic factor may be endogenous to the cell receiving the DSB agent. In some aspects, the morphogenic factor may be endogenous to a cell other than that receiving the DSB agent. In some aspects, the morphogenic factor is a heterologous molecule to the cell into which it is introduced. In some aspects, the morphogenic factor is provided on a particle. In some aspects, the morphogenic factor is provided as part of a transformation vector. In some aspects, the morphogenic factor is provided as a sequence outside of the T-DNA borders on a transformation vector. In some aspects, the introduction of the morphogenic factor to the cell results in transient association of the morphogenic factor with the cell. In some aspects, the introduction of the morphogenic factor to the cell results in the morphogenic factor becoming stably integrated into the genome of the recipient cell.

The guide polynucleotides, Cas endonucleases, polynucleotide modification templates, donor DNAs, guide polynucleotide/Cas endonuclease systems disclosed herein, and any one combination thereof, optionally further comprising one or more morphogenic factors, further comprising one more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

The introduction of a morphogenic factor into a cell can enhance the rate of targeted integration of a polynucleotide of interest. In these methods, at least one morphogenic factor is introduced into a cell and a double-strand break-inducing agent is introduced, along with a transfer cassette comprising the polynucleotide of interest. As used herein, a “transfer cassette” refers to a polynucleotide that can be introduced into a cell, wherein the polynucleotide comprises a polynucleotide of interest that is to be inserted into a target site of a cell. The introduction of a double-strand break can result in the integration of the polynucleotide of interest through non-homologous end joining or if the transfer cassette comprises at least one region of homology to the target site, the polynucleotide of interest can be integrated through homologous recombination.

A double-strand-break-inducing agent polynucleotide may be provided in expression cassettes for expression in a cell of interest. The cassette can include 5′ and 3′ regulatory sequences operably linked to an endonuclease polynucleotide or functional variant or functional fragment thereof. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally comprise at least one additional gene to be co-transformed into the organism. Alternatively, the additional gene(s) can be provided on multiple expression cassettes. Such an expression cassette is provided with a plurality of restriction sites and/or recombination sites for insertion of the endonuclease polynucleotide or functional variant or functional fragment thereof to be under the transcriptional regulation of the regulatory regions. The expression cassette may additionally comprise one or more selectable marker gene(s).

In some aspects, the DSB agent comprises a Cas endonuclease that is in a functional combination with a guide polynucleotide, that is capable of recognizing, binding to, and cleaving or nicking a target polynucleotide. In some aspects, the functional combination is a ribonucleoprotein complex.

In one embodiment, the expression constructs of the disclosure comprise a promoter operably linked to a nucleotide sequence encoding a Cas gene (or plant optimized, including a Cas endonuclease gene described herein) and a promoter operably linked to a guide RNA of the present disclosure. The promoter is capable of driving expression of an operably linked nucleotide sequence in a prokaryotic or eukaryotic cell/organism. In some aspects, the polynucleotides encoding the Cas gene and the guide RNA are on the same transformation vector. In some aspects, the polynucleotides encoding the Cas gene and the guide RNA are provided on different vectors. In some aspects, the polynucleotides encoding the Cas gene and the guide RNA are provided to the target cell concurrently. In some aspects, the polynucleotides encoding the Cas gene and the guide RNA are provided to the target cell sequentially.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

In some aspects, the Cas endonuclease and guide polynucleotide complex effects a double-strand break in a target polynucleotide, that results in a single base insertion, deletion, or modification (SDN1). In some aspects, the complex effects a double-strand break in a target polynucleotide, and a donor polynucleotide may change the sequence of the target (SDN2). In some aspects, the complex effects a double-strand break in a target polynucleotide, and new genetic material may be added into the break site (SDN3).

DNA double-strand breaks appear to be an effective factor to stimulate homologous recombination pathways (Puchta et al., (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al., (1995) Plant Mol Biol 28:281-92). In maize protoplasts, experiments with linear DNA molecules demonstrated enhanced homologous recombination between plasmids (Lyznik et al., (1991)Mol Gen Genet 230:209-18).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

Alteration of the genome of a prokaryotic and eukaryotic cell or organism cell, for example, through homologous recombination (HR), is a powerful tool for genetic engineering. Homologous recombination has been demonstrated in plants (Halfter et al., (1992)Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., Scientific American Books distributed by WH Freeman & Co.).

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct.

The donor DNA construct further comprises a first and a second region of homology that flank the polynucleotide of interest. The first and second regions of homology of the donor DNA share homology to a first and a second genomic region, respectively, present in or flanking the target site of the cell or organism genome.

In some aspects, the donor DNA is provided to the target cell on the same transformation construct as the polynucleotide encoding either the Cas9 and the guide RNA, or on the same construct as the polynucleotides encoding both the Cas9 and the guide RNA. In some aspects, the donor DNA is provided to the target cell on a different transformation construct as the polynucleotide encoding either the Cas9 and the guide RNA.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

In some aspects, the Cas endonuclease is part of a Cleavage Ready Cascade (crCascade) Complexes. Following characterization of the guide RNA and PAM sequence, components of the cleavage ready Cascade (crCascade) complex and associated CRISPR RNA (crRNA) may be utilized to modify chromosomal DNA in other organisms including plants. To facilitate optimal expression and nuclear localization (for eukaryotic cells), the genes comprising the crCascade may be optimized as described in WO2016186953 published 24 Nov. 2016, and then delivered into cells as DNA expression cassettes by methods known in the art. The components necessary to comprise an active crCascade complex may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 Apr. 2017), or any combination thereof. Additionally, a part or part(s) of the crCascade complex and crRNA may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof. To produce crRNAs in-vivo, tRNA derived elements may also be used to recruit endogenous RNAses to cleave crRNA transcripts into mature forms capable of guiding the crCascade complex to its DNA target site, as described, for example, in WO2017105991 published 22 Jun. 2017. crCascade nickase complexes may be utilized separately or concertedly to generate a single or multiple DNA nicks on one or both DNA strands. Furthermore, the cleavage activity of the Cas endonuclease may be deactivated by altering key catalytic residues in its cleavage domain (Sinkunas, T. et al., 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

As described herein, once the appropriate guide RNA requirement is established, the PAM preferences for each new system disclosed herein may be examined. If the cleavage ready Cascade (crCascade) complex results in degradation of the randomized PAM library, the crCascade complex can be converted into a nickase by disabling the ATPase dependent helicase activity either through mutagenesis of critical residues or by assembling the reaction in the absence of ATP as described previously (Sinkunas, T. et al., 2013, EMBO J 32:385-394). Two regions of PAM randomization separated by two protospacer targets may be utilized to generate a double-stranded DNA break which may be captured and sequenced to examine the PAM sequences that support cleavage by the respective crCascade complex.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/Cas endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 Sep. 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 Mar. 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

Thus, the disclosed methods and compositions can further comprise compositions and methods for the directional, targeted integration of exogenous nucleotides into a transformed plant are provided. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the transfer cassette.

It is recognized that the transformed plant prepared in this manner may comprise multiple target sites; i.e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

### Cells and Organisms

The methods and composition of the present disclosure may be used to improve genomic polynucleotide modification of a cell or organism.

In some aspects, the cell or organism is a prokaryote, for example but not limited to E. coli.

In some aspects, the cell or organism is from the Animal kingdom, for example a mammalian cell, for example a human cell. In some aspects, the cell is selected from the group consisting of: mouse connective tissue cell, mouse fibroblast, mouse embryonic stem cell, mouse monocyte, mouse macrophage, mouse spleen cell, mouse 3T3 NIH cell, mouse L cell, rat fibroblast, rat hepatoma, human lymphoma cell, human keratinocyte, human small cell lung cancer cell, human lymphocyte EBV transformed, human embryonic kidney cell, HEK293 cell, Chinese hamster ovary (CHO) cell, feline kidney cell, African green monkey kidney cell, SV 40 transformed cell, African monkey kidney cell, canine primary hepatocyte, chick embryonic fibroblast cell, HeLa cell, myeloma cell, bovine fetal heart cell, human egg, mouse egg, Xenopus egg, bovine egg, porcine egg, sheep egg, sheep or bovine udder epithelial cell, sheep embryonic epidermal cell, mouse blastocyst, stem cells, Syrian hamster kidney cell fibroblasts BHK-1 cell, monkey kidney epithelial cell BSC, mouse myeloma lymphoid cell MPC, frog egg cells RHP, and human nasopharyngeal tumor KB cell.

In some aspects, the cell or organism is from the Plant kingdom. In some aspects, the plant is a monocot. In some aspects, the plant is a dicot. Examples of plant species of interest include, but are not limited to, Arabidposis, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), teff (Eragrostis tef), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (crRNA+tracrRNA molecules) in said cell. The specific promoter can be, but is not limited to, a RNA polymerase III promoter, which allow for transcription of RNA with precisely defined, unmodified, 5′- and 3′-ends (Ma et al., 2014, Mol. Ther. Nucleic Acids 3:e161; DiCarlo et al., 2013, Nucleic Acids Res. 41: 4336-4343; WO2015026887, published 26 Feb. 2015). Any promoter capable of transcribing the guide RNA in a cell can be used and includes a heat shock/heat inducible promoter operably linked to a nucleotide sequence encoding the guide RNA.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 Apr. 2017.

Introducing a guide RNA/Cas endonuclease complex described herein, into a cell includes introducing the individual components of said complex either separately or combined into the cell, and either directly (direct delivery of a ribonucleoprotein complex, or RNP, as RNA for the guide and protein for the Cas endonuclease and Cas protein subunits, or functional fragments thereof) or via recombination constructs expressing the components (guide RNA, Cas endonuclease, Cas protein subunits, or functional fragments thereof). Introducing a guide RNA/Cas endonuclease complex (RNP) into a cell includes the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one Cas protein subunits) can be assembled in vitro or assembled by any means known in the art prior to being introduced into a cell (targeted for genome modification as described herein).

Plant cells differ from human and animal cells in that plant cells comprise a plant cell wall which may act as a barrier to the direct delivery of the RNP and/or of the direct delivery of the RNP components.

In some aspects, direct delivery of the RNP into plant cells can be achieved through particle mediated delivery (particle bombardment). Based on the experiments described herein, a skilled artesian can envision that any other direct method of delivery, such as but not limiting to, polyethylene glycol (PEG)-mediated transfection to protoplasts, electroporation, cell-penetrating peptides, or mesoporous silica nanoparticle (MSN)-mediated direct protein delivery, can be successfully used for delivering RNP into plant cells.

Direct delivery of the RNP, allows for genome editing at a target site in the genome of a cell which can be followed by rapid degradation of the complex, and only a transient presence of the complex in the cell. This transient presence of the RNP complex may lead to reduced off-target effects. In contrast, delivery of RNP components (guide RNA, Cas endonuclease) via plasmid DNA sequences can result in constant expression of RNPs from these plasmids which can intensify off target effects (Cradick, T. J. et al. (2013) Nucleic Acids Res 41:9584-9592; Fu, Yet al. (2014) Nat. Biotechnol. 31:822-826).

Direct delivery can be achieved by combining any one component of the guide RNA/Cas endonuclease complex (RNP) (such as at least one guide RNA, at least one Cas protein, and at least one Cas protein), with a particle delivery matrix comprising a microparticle (such as but not limited to of a gold particle, tungsten particle, and silicon carbide whisker particle) (see also WO2017070032 published 27 Apr. 2017).

In one aspect, the guide polynucleotide/Cas endonuclease complex is a complex wherein the guide RNA and Cas endonuclease protein forming the guide RNA/Cas endonuclease complex are introduced into the cell as RNA and protein, respectively.

In one aspect, the guide polynucleotide/Cas endonuclease complex is a complex wherein the guide RNA and Cas endonuclease protein and the at least one protein subunit of a Cas protein forming the guide RNA/Cas endonuclease complex are introduced into the cell as RNA and proteins, respectively.

In one aspect, the guide polynucleotide/Cas endonuclease complex is a complex wherein the guide RNA and Cas endonuclease protein and the at least one protein subunit of a Cascade forming the guide RNA/Cas endonuclease complex (cleavage ready cascade) are preassembled in vitro and introduced into the cell as a ribonucleotide-protein complex.

Protocols for introducing polynucleotides, polypeptides or polynucleotide-protein complexes into eukaryotic cells, such as plants or plant cells are known and include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher: InTech, Rijeka, Croatia. CODEN: 69PQBP; ISBN: 978-953-307-201-2), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, New York), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

The methods and composition of the disclosure can utilize a variety of transformation methods as appropriate. That is, transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides introduced into an explant by the disclosed methods and compositions can be operably linked to a suitable promoter. “Promoter” means a region of DNA that is upstream from the start of transcription and is involved in recognition and binding of RNA polymerase and other proteins to initiate transcription, either including or not including the 5′ UTR. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as from Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissues are referred to as “tissue specific”. A “cell type” specific promoter primarily drives expression in certain cell types in one or more organs, for example, vascular cells in roots or leaves. An “inducible” or “repressible” promoter can be a promoter which is under either environmental or exogenous control. Examples of environmental conditions that may affect transcription by inducible promoters include anaerobic conditions, or certain chemicals, or the presence of light. Alternatively, exogenous control of an inducible or repressible promoter can be affected by providing a suitable chemical or other agent that via interaction with target polypeptides result in induction or repression of the promoter. Tissue specific, tissue preferred, cell type specific, and inducible promoters constitute the class of “non-constitutive” promoters. A “constitutive” promoter is a promoter which is active under most conditions. Promoters useful in the present disclosure include those disclosed in WO2017/112006 and those disclosed in U.S. Provisional Application 62/562,663.

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. “Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The expression cassette can include in the 5′-3′ direction of transcription, a transcriptional and translational initiation region (i.e., a promoter), an endonuclease polynucleotide or functional variant or functional fragment thereof, and a transcriptional and translational termination region (i.e., termination region) functional in the recipient organism. The regulatory regions (i.e., promoters, transcriptional regulatory regions, and translational termination regions) and/or the endonuclease polynucleotide or functional variant or functional fragment thereof may be native/analogous to the host cell or to each other. Alternatively, the regulatory regions and/or the endonuclease polynucleotide of or functional variant or functional fragment thereof may be heterologous to the host cell or to each other.

While it may be desired in some embodiments to express the sequences using heterologous promoters, the native promoter sequences to the polynucleotide encoding the double-strand-break-inducing agent may alternatively be used. Such constructs can change expression levels of the polynucleotide in the cell. Thus, the phenotype of recipient cell can be altered.

Where appropriate, the polynucleotides may be optimized for increased expression in the recipient cell or organism. That is, the polynucleotides can be synthesized using organism-preferred codons for improved expression. See, for example, Campbell and Gowri (1990) Plant Physiol. 92:1-11 for a discussion of host-preferred codon usage in plants.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, sulfonylureas, dicamba, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad Aci. USA 86:5400-5404; Fuerst et al. (1989) Proc. Natl. Acad Sci. USA 86:2549-2553; Deuschle et al. (1990) Science 248:480-483; Gossen (1993) Ph.D. Thesis, University of Heidelberg; Reines et al. (1993) Proc. Natl. Acad Sci. USA 90:1917-1921; Labow et al. (1990) Mol. Cell. Biol. 10:3343-3356; Zambretti et al. (1992) Proc. Natl. Acad Sci. USA 89:3952-3956; Baim et al. (1991) Proc. Natl. Acad Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. The above list of selectable marker genes is not meant to be limiting. Any selectable marker gene may be used with any of the methods or compositions disclosed herein.

In some aspects, the DSB agent may be introduced into a target cell and the morphogenic factor may be introduced into the same target cell.

In some aspects, the DSB agent may be introduced into a target cell and the morphogenic factor may be introduced into a different cell. In some aspects, the target cell and the different cell may each comprise the same target polynucleotide. In some aspects, the different cell is an “adjacent” cell. As used herein, the term “adjacent cell” (or the plural “adjacent cells”) is used to describe a cell that is part of the same organism, the same tissue, in the same cell culture, or in the same cell layer as the target cell, but is not the same cell as the target cell. In some aspects, the adjacent cell is in physical contact with the target cell. In some aspects, the adjacent cell is separated from the target cell, for example by at another cell.

### Traits of Agronomic Importance

The methods and compositions provided herein, which provide for improved efficacy of DSB agent activity on a target polynucleotide in a cell by use of a morphogenic factor, are useful for the production of organisms, including plants, with desirable characteristics including traits of importance.

As used herein, “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance. An “enhanced trait” as used in describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, enhanced nitrogen use efficiency, early plant growth and development, late plant growth and development, enhanced seed protein, and enhanced seed oil production.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

### Aspects

Aspect 1: A method of editing a polynucleotide in the genome of a target cell, comprising: (a) providing a double-strand-break-inducing agent to the target cell, and (b) providing a morphogenic factor to an adjacent cell.

Aspect 2: A method of editing a polynucleotide in the genome of a target cell, comprising: (a) providing a double-strand-break-inducing agent to the target cell, and (b) upregulating the transcription of an endogenous morphogenic factor gene in the genome of said target cell or in the genome of an adjacent cell.

Aspect 3: A method of editing a polynucleotide in the genome of a target cell, comprising: (a) providing a double-strand-break-inducing agent into the target cell, (b) providing to the target cell or to an adjacent cell a T-DNA vector comprising a gene for a morphogenic factor outside of said left border and said right border.

Aspect 4: A method of editing a polynucleotide in the genome of a target cell, comprising: (a) providing to the target cell a double-strand-break-inducing agent, and (b) providing a morphogenic factor to the target cell or to an adjacent cell; wherein either the double-strand-break-inducing agent of (a) or the morphogenic factor of (b) or both further comprise(s) a cell penetrating peptide motif at its 3′ end, its 5′ end, or both ends.

Aspect 5: A method of editing a polynucleotide in the genome of a target cell, comprising: (a) providing to a cell a first vector comprising a polynucleotide encoding a double-strand-break-inducing agent, and (b) providing a morphogenic factor to the target cell or to an adjacent cell; wherein the first vector further comprises outside of the T-DNA borders a repressor fused to a gene encoding a Cas endonuclease that lacks nuclease activity, wherein said repressor is capable of binding to a regulatory element operably linked to the polynucleotide encoding the double-strand-break-inducing agent of (a).

Aspect 6: A method of editing a polynucleotide in the genome of a target cell, comprising: (a) providing to the target cell a double-strand-break-inducing agent, (b) providing to a cell a composition selected from the group consisting of: i. a molecule that stimulates the expression of an endogenous morphogenic factor in the cell, and ii. a T-DNA vector comprising a gene for a morphogenic factor outside of said left border and said right border, wherein the cell of (a) is the same cell as the cell of (b), and wherein either the double-strand-break-inducing agent of (a) or the composition of (b) or both further comprise(s) a cell penetrating peptide motif at its 3′ end, its 5′ end, or both ends.

Aspect 7: A method of editing a polynucleotide in the genome of a target cell, comprising: (a) providing to the target cell a double-strand-break-inducing agent, (b) providing to a cell a composition selected from the group consisting of: i. a morphogenic factor, ii. a molecule that stimulates the expression of an endogenous morphogenic factor, and iii. a T-DNA vector comprising a polynucleotide of interest between the left border and the right border, further comprising a gene for a morphogenic factor outside of said left border and said right border; wherein the cell of (a) is different than the cell of (b), and wherein either the double-strand-break-inducing agent of (a) or the composition of (b) or both further comprise(s) a cell penetrating peptide motif at its 3′ end, its 5′ end, or both ends.

Aspect 8: The method of any of Aspects 1-6, wherein the morphogenic factor is selected from the group consisting of: Wuschel, Ovule Development Protein, and Babyboom.

Aspect 9: The method of any of Aspects 1-6, wherein the composition of (a) is provided on a different construct than the composition of (b).

Aspect 10: The method of any of Aspects 1-6, wherein the composition of (a) is provided on the same construct as the composition of (b).

Aspect 11: The method of any of Aspects 1-6, wherein the morphogenic factor is provided as a polynucleotide sequence encoding a polypeptide.

Aspect 12: The method of any of Aspects 1-6, wherein the morphogenic factor shares at least 80% identity with a sequence selected from the group consisting of: SEQID NOs: 6-10, 17-21, and 48-73.

Aspect 13: The method of any of Aspects 1-6, wherein the morphogenic factor is provided as a polypeptide.

Aspect 14: The method of any of Aspects 1-6, wherein the morphogenic factor is encoded by a polynucleotide that shares at least 80% identity with a sequence selected from the group consisting of: SEQID NOs:1-5, 11-16, 22, and 23-47.

Aspect 15: The method of any of Aspects 1-6, wherein the morphogenic factor is selected from the group consisting of: WUS1, WUS2, WUS3, WOX2A, WOX4, WOX5, WOX9, BBM2, BMN2, BMN3, and ODP2.

Aspect 16: The method of any of Aspects 1-6, wherein said editing is selected from the group consisting of: insertion of at least one polynucleotide, deletion of at least one polynucleotide, modification of at least one polynucleotide, substitution of at least one polynucleotide, and a combination of at least two of the preceding.

Aspect 17: The method of any of Aspects 1-6, wherein said double-strand-break-inducing agent comprises a Cas endonuclease, a TALEN, a meganuclease, a zinc-finger endonuclease, or a restriction endonuclease.

Aspect 18: The method of any of Aspects 1-6, wherein said double-strand-break-inducing agent is provided as a ribonucleoprotein complex comprising a Cas endonuclease protein and a guide RNA.

Aspect 19: The method of any of Aspects 1-6, further comprising introducing a heterologous polynucleotide donor DNA molecule into said target cell.

Aspect 20: The method of any of Aspects 1-6, wherein the morphogenic factor is operably linked to a heterologous promoter.

Aspect 21: The method of Aspect 19, wherein said promoter is selected from the group consisting of: a ZM-PLTP promoter, a ZM-PLTP1 promoter, a ZM-PLTP2 promoter, a SB-PLTP1 promoter, a SB-PLTP2 promoter, a SB-PLTP3 promoter, an OS-PLTP1 promoter, an OS-PLTP2 promoter, a SI-PLTP1 promoter, a ZM-FBP1 promoter, a ZM-RFP promoter, a ZM-APMP promoter, a ZM-RfeSP promoter, a ZM-CRR6 promoter, a ZM-G3K promoter, a ZM-CAB7 promoter, a ZM-UBR promoter, a ZM-HBP promoter, a ZM-PS1-N promoter, a ZM-SDR promoter, an OS-SDR promoter, a SB-SDR promoter, a ZM-SDR (long) promoter, a ZM-LGL promoter, a ZM-LEA14-A promoter, a ZM-LEA34-D promoter, a GM-LTP3 promoter, a GM-EF1A promoter, a GM-HBSTART3 promoter, a GM-HBSTART3 (TRUNCATED) promoter, an AT-ML1 promoter, a GM-ML1-Like promoter, a GM-ML1-Like (TRUNCATED) promoter, a ZM-HBSTART3 promoter, an OS-HBSTART3 promoter, an AT-PDF1 P2 promoter, a GM-PDF1 promoter, a GM-PDF1 (TRUNCATED) promoter, a SB-PDF1 promoter, an OS-PDF1 promoter, an OS-PDF1 (TRUNCATED) promoter, a PT-PDF1 promoter, a PT-PDF1 (TRUNCATED) promoter, a SI-PDF1 promoter, a SI-PDF1 (TRUNCATED) promoter, an AT-PDF2 promoter, a GM-PDF2 promoter, a GM-PDF2 (TRUNCATED) promoter, a ZM-GL1 promoter, an AT-PDF2a promoter, an AT-PDF2a (TRUNCATED) promoter, a GM-PDF2a promoter, a GM-PDF2a (TRUNCATED) promoter, an OS-PDF2 promoter, an OS-PDF2 (TRUNCATED) promoter, a PT-PDF2 promoter, a PT-PDF2 (TRUNCATED) promoter, a VV-PDF2 promoter, a VV-PDF2 (TRUNCATED) promoter, aZM-PDF2 promoter, a SI-PDF2 promoter, a SI-PDF2 (TRUNCATED) promoter, aVV-PDF2a promoter, a PT-PDF2a promoter, a PT-PDF2a (TRUNCATED) promoter, a MT-PDF2 promoter, a MT-PDF2 (TRUNCATED) promoter, an AT-HDG2 promoter, a GM-HDG2 promoter, a GM-HDG2 (TRUNCATED) promoter, a SB-HDG2 promoter, a SB-HDG2 (TRUNCATED) promoter, an AT-CER6 promoter, an AT-CER60 promoter, an AT-CER60 (TRUNCATED) promoter, a GM-CER6 promoter, a GM-CER6 (TRUNCATED) promoter, a PT-CER6 promoter, a PT-CER6 (TRUNCATED) promoter, a VV-CER6 promoter, a VV-CER6 (TRUNCATED) promoter, a SB-CER6 promoter, a ZM-CER6 promoter, a SI-CER6 promoter, a SI-CER6 (TRUNCATED) promoter, an OS-CER6 promoter, an OS-CER6 (TRUNCATED) promoter, a GM-HBSTART2 promoter, a GM-MATE1 promoter, a GM-NED1 promoter; a SB-GL1 promoter, an OS-GL1 promoter, an AT-GL1 promoter, a GM-GL1 promoter, an AT-ANL1 promoter, a ZM-OCL1 promoter, and an OS-OCL1 promoter.

Aspect 22: The method of Aspect 18, wherein said donor DNA molecule comprises a polynucleotide that confers a benefit to an organism comprising, or derived from, said target cell.

Aspect 23: The method of Aspect 21, wherein said benefit is selected from the group consisting of: improved health, improved growth, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction.

Aspect 24: The method of any of Aspects 1-6, wherein said cell is a plant cell.

Aspect 25: The method of Aspect 23, wherein said plant cell is obtained from or derived from a monocot or dicot plant.

Aspect 26: The method of Aspect 24, wherein the monocot selected from the group consisting of: Zea mays, Sorghum bicolor, Sorghum vulgare, Triticum aestivum, Medicago sativa, Oryza sativa, Setaria italica, and Saccharum spp.

Aspect 27: The method of Aspect 24, wherein the dicot selected from the group consisting of: Helianthus annuus, Glycine max, Nicotiana tabacum, Gossypium barbadense, Gossypium hirsutum, Manihot esculenta, Beta vulgaris, Brassica spp., and Arabidposis thaliana.

Aspect 28: The method of any of Aspects 1-6, wherein said editing of a polynucleotide in the genome of the cell modulates a trait of agronomic importance in a plant, selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition; as compared to an isoline plant not comprising or derived from a cell whose genome was edited with said double-strand-break-inducing agent.

Aspect 29: A plant derived from a plant cell produced by the method of any of Aspects 1-6, wherein said plant does not have a heterologous morphogenic factor stably integrated into its genome.

Aspect 30: Any of the preceding aspects, wherein the morphogenic factor is engineered or synthetic, wherein the engineered or synthetic morphogenic factor has an altered type or level of activity as compared to a non-engineered or non-synthetic morphogenic factor.

Aspect 31: Any of the preceding aspects, wherein the Cas endonuclease is provided as a polypeptide, or wherein the Cas endonuclease is provided as a polynucleotide encoding a Cas endonuclease polypeptide, or wherein the Cas endonuclease is provided as a ribonucleoprotein complex with a guide polynucleotide.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

### Example 1: Particle Bombardment Transformation of Corn Plants

In this example, transformation of immature maize embryos via particle bombardment is described. It is understood that a similar protocol may be used for the transformation of other plants, such as (but not limited to): soybean, cotton, canola, wheat, rice, sorghum, or sunflower.

Prior to bombardment, 10-12 DAP immature embryos are isolated from ears of a corn plant and placed on culture medium plus 16% sucrose for three hours to plasmolyze the scutellar cells.

Single plasmids or multiple plasmids may be used for each particle bombardment. In one example, a plurality of plasmids include: 1) a plasmid comprising a donor cassette, 2) a plasmid comprising an expression cassette for the double-strand-break-inducing agent, and 3) a plasmid comprising an expression cassette for the morphogenic factor.

To attach the DNA to 0.6 μm gold particles, the plasmids are mixed by adding 10 μl of each plasmid together in a low-binding microfuge tube (Sorenson Bioscience 39640T). To this suspension, 50 μl of 0.6 μm gold particles (30 μg/μl) and 1.0 μl of Transit 20/20 (Cat No MIR5404, Mirus Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all the remaining ethanol. Particle bombardment is performed using a Biolistics PDF-1000, at 28 inches of Mercury using a 200 PSI rupture disc.

After particle bombardment, the immature embryos are selected on 506J medium modified to contain 12.5 g/l mannose and 5 g/l maltose and no sucrose. After 10-12 weeks on selection, plantlets are regenerated and analyzed using qPCR.

### Example 2: Agrobacterium-Mediated Transformation of Corn Plants

In this example, transformation of immature maize embryos via Agrobacterium mediation is described. It is understood that a similar protocol may be used for the transformation of other plants, such as (but not limited to): soybean, cotton, canola, wheat, rice, sorghum, or sunflower.

### Preparation of Agrobacterium Master Plate.

Agrobacterium tumefaciens harboring a binary donor vector is streaked out from a −80° C. frozen aliquot onto solid 12V medium and cultured at 28° C. in the dark for 2-3 days to make a master plate.

### Growing Agrobacterium on Solid Medium.

A single colony or multiple colonies of Agrobacterium are picked from the master plate and streaked onto a second plate containing 810I medium and incubated at 28° C. in the dark overnight.

Agrobacterium infection medium (700 medium A; 5 ml) and 100 mM 3′-5′-Dimethoxy-4′-hydroxyacetophenone (acetosyringone; 5 μL) are added to a 14 mL conical tube in a hood. About 3 full loops of Agrobacterium from the second plate are suspended in the tube and the tube was then vortexed to make an even suspension. One mL is transferred to a spectrophotometer tube and the optical density (550 nm) of the suspension is adjusted to a reading of about 0.35-1.0. The Agrobacterium concentration is approximately 0.5 to 2.0×10⁹ cfu/mL. The final Agrobacterium suspension is aliquoted into 2 mL microcentrifuge tubes, each containing about 1 mL of the suspension. The suspensions are then used as soon as possible.

### Growing Agrobacterium on Liquid Medium.

Alternatively, Agrobacterium can be prepared for transformation by growing in liquid medium. One day before infection, a 125 ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate is suspended into the flasks and placed on an orbital shaker set at 200 rpm and incubated at the 28° C. overnight. The Agrobacterium culture is centrifuged at 5000 rpm for 10 min. The supernatant is removed and the Agrobacterium infection medium with acetosyringone solution was added. The bacteria are resuspended by vortex and the optical density (550 nm) of Agrobacterium suspension is adjusted to a reading of about 0.35 to 2.0.

### Maize Transformation.

Ears of a maize (Zea mays L.) cultivar are surface-sterilized for 15-20 min in 20% (v/v) bleach (5.25% sodium hypochlorite) plus 1 drop of Tween 20 followed by 3 washes in sterile water. Immature embryos (IEs) are isolated from ears and are placed in 2 ml of the Agrobacterium infection medium with acetosyringone solution. The optimal size of the embryos varies based on the inbred, but for transformation with WUS2 and ODP2 a wide size range of immature embryo sizes could be used. The solution is drawn off and 1 ml of Agrobacterium suspension was added to the embryos and the tube vortexed for 5-10 sec. The microfuge tube is allowed to stand for 5 min in the hood. The suspension of Agrobacterium and embryos are poured onto 7101 (or 562V) co-cultivation medium (see Table 2). Any embryos left in the tube are transferred to the plate using a sterile spatula. The Agrobacterium suspension is drawn off and the embryos placed axis side down on the media. The plate is sealed with Parafilm M® film (moisture resistant flexible plastic, available at Bemis Company, Inc., 1 Neenah Center 4^{th} floor, PO Box 669, Neenah, WI 54957) and incubated in the dark at 21° C. for 1-3 days of co-cultivation.

Embryos are transferred to resting medium (605T medium) without selection. Three to 7 days later, they are transferred to maturation medium (289Q medium) supplemented with a selective agent

### Example 3: Delivery of a Morphogenic Factor to an Adjacent Cell Improves CRISPR-Cas Mediated Polynucleotide Modification of Target Cells

This example demonstrates that delivery of DNA vector encoding for WUS protein on a separate set of gold particles into maize immature embryo cells is sufficient to support embryogenesis and plant regeneration with targeted mutations/deletions or site-specific insertions and no WUS gene integration with practical frequencies.

Previously, plant transformation and genome editing approaches required delivery of various DNA vectors coding for different “helper” components, including double-strand break reagents (for example, meganucleases, ZFNs, TALENs, or Cas9 nuclease and guide RNA (gRNA)), a selectable marker, morphogenic factors (e.g., ODP2 and WUS), in addition to the “donor” DNA—single-stranded or double-stranded oligonucleotides in gene editing experiments or plasmid DNA containing parts or entire genes with regulatory elements (promoters and terminators) for insertions as the result of double-strand break (DSB) through homology directed repair (HDR) pathway.

Multiple, co-delivered DNA molecules tend to co-integrate into a DSB site through the non-homologous end joining (NHEJ) repair pathway, significantly reducing the frequency of usable events. Moreover, stable integration of CRISPR components may lead to plant chimerism and increase chances of off-site mutagenesis. Minimizing the introduction of the described above “helper” DNA molecules into the target cell, and limiting delivery to the donor DNA, may be beneficial and lead to higher frequencies of quality events (QEs).

Previously, we have described a method for Cas9-gRNA delivery in the form of ribonucleoproteins (RNPs) using gold microparticles. We also described a method for activation of broken pre-integrated selectable marker gene through non-homologous end joining (NHEJ) mechanism upon Cas9-gRNA delivery as DNA vectors or RNP complex (WO 2017/070029 A1).

Morphogenic factor genes (also referred to as “developmental genes”, for example but not limited to: ODP2 and WUS) are desired components of the transformation process: their delivery into plant cells facilitates cell division and significantly increases transformation frequencies. Moreover, these genes allow successful transformation of many elite genotypes, which transformation, otherwise, cannot be effectively accomplished. However, stable integration of morphogenic factor genes (e.g., ODP2 and WUS) into the target cell genome may have deleterious effect on plant regenerability and fertility.

Here we describe an approach allowing prevention of morphogenic factor (developmental genes, or “dev genes”) integration into the genomes of regenerated plants. First, we demonstrated that delivery of only one of the two morphogenic factor genes (WUS) under a strong promoter (for example, PLTP) was sufficient to stimulate embryogenesis in most tested genotypes. Second, WUS protein has a cell-penetrating peptide (CPP) and therefore has an ability to penetrate cell walls. Thus, the delivery of vector DNA encoding for WUS into a cell led to protein expression and its migration to the neighboring cells stimulating their division. Moreover, overexpression of the protein in the targeted cell is usually toxic preventing the call division, embryogenesis and plant regeneration. Based on these observations, we conducted biolistic delivery of RNP and donor DNA on the first set of gold particles (first shot) followed by delivery of vector DNA containing WUS expression cassette on a separate set of gold particles (second shot). This allowed plant regeneration from cells receiving the DSB and editing components and stimulated to division and embryogenesis by WUS protein molecules coming from adjacent cells.

### Gene Deletion

A gene deletion of approximately 21 kb in size was generated. Maize immature embryos were bombarded using gold particles coated with 2 RNP complexes targeting two sites:

| | site 1 |
|---|---|
| | (PAM: CGG) |
| SEQ ID NO: 80 | |
| | GGATTCCGCGGAAATGGGTG |
| | site 2 |
| | (PAM: AGG) |
| SEQ ID NO: 81 | |
| | GTCAAGGACATACGAGACC |

to generate the deletion and then immediately bombarded embryos with the second set of gold particles coated with a DNA vector encoding for WUS (polynucleotide SEQID NO: 23 encoding polypeptide SEQID NO:48) operably linked to a PLTP promoter (SEQID NO:74) (FIG. 1A). Plants were regenerated without selection and analyzed by PCR and sequencing, and qPCR for the presence of deletions and WUS integration, respectively. Results of this experiment are presented in Table 2.

TABLE 2
Frequency of RecQ4 gene deletion

| | | Plants | Plants with | Plants with deletion |
|---|---|---|---|---|
| Embryos | Plants | with | WUS | No WUS |
| bombarded | sampled | deletion | integration | integration |
| 385 | 1128 | 54 (5%) | 34 (3%) | 51 |

These results demonstrate efficient, “DNA-free” gene deletion with high efficiency.

### Site-Specific Gene Insertion

Separate WUS delivery was used to generated a gene insertion of approximately 3.5 kb in size to a specific genomic target site. Maize immature embryos were bombarded using gold particles co-coated with a DNA vector comprising polynucleotides encoding an optimized S. pyogenes Cas9 protein (exons 1 and 2 given as polynucleotide SEQID NOs: 103-104 encoding polypeptide SEQID NOs: 108-109, with a potato LS1 intron inserted between the two exons, and further comprising SV40 and VIRD2 NLS sequences) and an appropriate gRNA (SEQID NO: 82) (FIG. 1i), and a DNA vector comprising a selectable marker gene (NPTII, polynucleotide SEQID NO: 77 encoding polypeptide SEQID NO:100) flanked with homology arms (SEQID NOs: 78 and 79) as donor DNA (FIG. 1C). The donor DNA comprised a gene of interest flanked with sequences homologous to the genomic sequences on both sides of the DSB (homology arms). The same embryos were then bombarded a second time, delivering a DNA vector encoding for WUS under the PLTP promoter (FIG. 1A). Plants were regenerated on media containing a selective agent (G418), which is toxic to cells that have not integrated and express the NPTII gene. Plants were analyzed by junction PCR for integration of the gene of interest into the targeted genomic site. Regenerants were also tested by qPCR for the presence of WUS. Results of the experiment are presented in Table 3.

TABLE 3
Frequency of NPTII targeted gene integration generated

| | | | Plants | Plants with NPTII |
|---|---|---|---|---|
| | | Plants with | with NPTII | insertion, |
| Embryos | Plants | targeted | insertion, WUS | No WUS |
| bombarded | sampled | integration | integration | integration |
| 305 | 115 | 10 (8.7%) | 6 (60%) | 4 (40%) |

Separate delivery of WUS was sufficient to promote plant regeneration from cells with targeted mutations/deletions, gene edits or gene insertions and, therefore, replaced co-delivery of morphogenic factor genes (ODP2 and/or WUS) along with other components. Thus, plants regenerated in these experiments didn't contain morphogenic factor genes randomly integrated into the genome, thus improving regenerability and fertility of the plants.

This method of separate delivery of a morphogenic factor gene facilitated a “DNA-free” approach to gene knockout, targeted gene editing, and gene insertion in plant cells of agronomically important crop plants including but not limited to maize, soybean, wheat, rice, millet, sorghum, cotton, and canola.

### Example 4: Activation of Endogenous Morphogenic Factors Improves CRISPR-Cas Mediated Polynucleotide Modification of Target Cells

In this example, a method is provided for enabling plant transformation using the targeted overexpression of a developmental gene using an RNA programmable homing endonuclease-transcriptional activator complex. The basic platform this is built upon is the CRISPR/Cas9 system (specifically S. pyogenes, abbreviated Spy Cas9) which is capable of being directed to any target sequence with the modifications of removing its endonuclease activity and combining it with a known transcriptional activator motif. In this case, the D10A and H840A mutations in Spy Cas9 were made (polynucleotide SEQID NO:105 encoding polypeptide SEQID NO:110) and the transcriptional activator component of Arabidopsis CBF1 protein (referred to as AT-CBF1A, polynucleotide SEQID NO:83 encoding polypeptide SEQID NO:101) was fused. This method is a novel advancement to the method of plant transformation and creation of polynucleotide modifications in cell genomes via a double-strand-break-inducing agent, in which manipulation of the endogenous gene's expression is enhanced.

To test the ability of a dSpy-Cas9 and guides expressed from bombarded plasmids, pol III (promoter polynucleotide SEQID NO:84) expression cassettes with guides targeting the Variety 1 promoter region of WUS2 were developed. FIG. 2A shows the general location of the 5 effective guides (UTRs and exon/intron structures based on the maize B73 version of the gene, using genomic maize Variety 1 contig sequences, with markups representing the allele differences.

guide sequences used in expression cassette construction:

| | C5 |
|---|---|
| (SEQ ID NO: 86) | |
| | GCCTTTGCAGTTTGCACC |
| | C3 |
| (SEQ ID NO: 87) | |
| | GTTGCCACAAGGGGAGCC |
| | C9 |
| (SEQ ID NO: 88) | |
| | GCAAATGACTTCTGTCTCTA |
| | A9 |
| (SEQ ID NO: 89) | |
| | GACTCTTCCAAATTTCGAAG |
| | A4 |
| (SEQ ID NO: 90) | |
| | GTCGTATCACCCATGGGCAA |

The map of the dCas9-GRA-CBF1A vector is shown in FIG. 2B. The GRA sequence was: GGCAGGGCT.

Different combinations of guides were delivered into Variety 1 along with NPT II selection marker and dCas9-GRA-CBF1A and were monitored for embryonic stem cell growths. At 4 weeks post bombardment the embryos were analyzed. Notably, the 3 sense C guides and all 5 of the A and C sense guides gave strong responses. The antisense guides did not provide transformation enhancement (FIG. 2C).

The experiment was repeated with full growth with NPTII selection until plants developed using only the 3 sense C guides and the 5 sense A+C guides. FIG. 2D shows plants after 5-7 weeks of outgrowth.

FIG. 2E shows the protein sequence differences between the WUS2 from a recombinant vector (polynucleotide SEQID NOs: 73 (exon 1) and 24 (exon 2) encoding polypeptide SEQID NO:49), from a native B73 maize plant (polynucleotide SEQID NO:25 encoding polypeptide SEQID NO:50), and from a native Variety 1 maize plant (polynucleotide SEQID NO:26 encoding polypeptide SEQID NO:51).

As shown in Table 4, fertile TO plants were recovered from the plants. “Total recovered” was the number of plantlets successfully generated in the transformation lab through NPTII selection and sent to greenhouse. “Number discarded” refers to those that were observed from the greenhouse to be off phenotype, runts, sterile, or died. “Pollinated” refers to the number of plants that either via carry-in or carry-out gave >10 seed number.

TABLE 4
Recovery of Fertile T0s from Endogenous
Morphogenic Factor Activation

| | 3 guides | 5 guides | PLTP:wus | no DevGenes |
|---|---|---|---|---|
| total recovered | 30 | 31 | 48 | 12 |
| number discarded | 8 | 11 | 24 | 6 |
| pollinated | 22 | 20 | 24 | 6 |
| % pollinated/recovered | 73.3% | 64.5% | 50% | 50% |

### Example 5: Morphogenic Factor Gene Placement Outside of the Transformation Cassette Borders Improves CRISPR-Cas Mediated Polynucleotide Modification of Target Cells

Three plasmids are prepared for Agrobacterium-mediated transformation as described above, each comprising the same T-DNA:
RB+Promoter::SelectableMarker::Terminator+Promoter::DSBagent::Terminator+LB but differing in the placement of the morphogenic factor expression cassettes beyond the left border.

Plasmid A comprises two expression cassettes immediately outside the Left Border:
Promoter::MorphogenicFactor1::Terminator
Promoter::MorphogenicFactor2::Terminator

Plasmid B comprises a single expression cassette:
Promoter::MorphogenicFactor::Terminator

Plasmid C comprises a single expression cassette outside the Left Border:
Promoter::MorphogenicFactorl+MorphogenicFactor2::Terminator

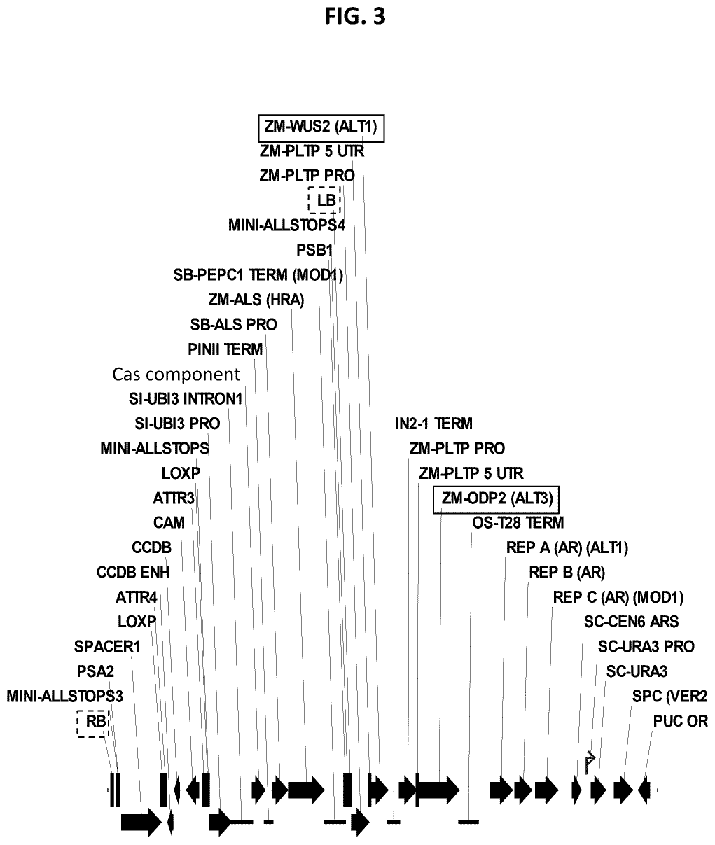

One example of a vector comprising two morphogenic factor genes (WUS2 given as polynucleotide SEQID NO:27 encoding polypeptide SEQID NO:52, and ODP2 given as polynucleotide SEQID NO:22 encoding polypeptide SEQID NO:17) outside of the left T-DNA border is shown in FIG. 3.

After Agrobacterium-mediated transformation with these respective T-DNAs and selection during somatic embryo maturation and regeneration, TO plants are analyzed by qPCR. Based on qPCR analysis, using Plasmid A results in 10.2% of the recovered TO plants being single copy for the DSB agent gene within the T-DNA, while the values for Plasmid B and Plasmid C were 12.2% and 12.3%, respectively, while lacking integrated morphogenic factor gene, with no additional indications of plasmid backbone contamination.

A cell transformed with any of these plasmids exhibit modifications of its genomic polynucleotide sequence as a result of the presence of the introduced DSB agent, without the heterologous morphogenic factor gene integrated into the genomic DNA.

These results demonstrate that positioning the morphogenic factor expression cassettes outside the T-DNA Left Border provides the necessary stimulation of transformation without the concomitant integration of these morphogenic factors into the genome of the target cell.

### Example 6: Enabling Morphogenic Factor Mobility Improves CRISPR-Cas Mediated Polynucleotide Modification of Target Cells

For this experiment, the maize WUS2 gene is modified through either replacing the WUS Cell Penetrating Peptide (CPP) motif with a KN1 CPP motif, or by leaving the WUS CPP unaltered and adding a KN1 CPP to the amino terminus of the protein. In both cases, when an expression cassette is used that expresses the wild-type WUS protein (e.g. PLTP PRO::WUS2::IN2-1 TERM) along with UBI::CAS9::PINII TERM+U6-POL PRO::gRNA::U6 TERM cassette where the gRNA is designed to guide the dCAS9 protein to bind and cut the endogenous maize ALS gene, edits of the ALS gene to confer chlorsulfuron resistance to the cells are readily recovered. However, when either the KN1-CPP is substituted for the WUS-CPP or the KN1-CPP is added to the WUS protein, more diffusion of the modified WUS proteins results in increased growth and a higher rate of recovering chlorsulfuron-resistant plants.

### Example 7: Using a Combination of WUS and REPA Results in Higher Rates of Cell Division and Concomitant CRISPR-Cas Mediated Polynucleotide Modification of Target Cells

For these experiments, two treatments are compared. In the first treatment, an Agrobacterium containing a T-DNA with the following components is used; In the first treatment, a single Agrobacterium is used containing a single T-DNA comprising the following components: RB+U6-POL PRO::gRNA::U6 TERM cassette where the gRNA is designed to guide the dCAS9 protein to bind and cut the endogenous maize ALS gene, ii) a UBI PRO::CAS9::PINII TERM, iii) an expression cassette containing a PLTP PRO::ZM-WUS2::IN2-1 TERM expression cassette (the Control Plasmid). In the second treatment, a mixture of two Agrobacteria are used; in the first Agrobacterium, a plasmid contains a binary vector with the following components within the T-DNA; i) a) a U6-POL PRO::gRNA::U6 TERM cassette where the gRNA is designed to guide the dCAS9 protein to bind and cut the endogenous maize ALS gene, ii) an expression cassette containing a PLTP PRO::ZM-WUS2::IN2-1 TERM expression cassette and a PLTP PRO::CAMV-REPA::IN2-1 TERM expression cassette. The second Agrobacterium contains a T-DNA with a sequence (not in an expression cassette) that will be used as the editing template for modifying the ALS gene (the Test Plasmid). The first and second Agrobacteria are mixed in a ratio of 9:1 and used for transformation.

After Agrobacterium-mediated transformation with Agrobacterium strain LBA4404 THY—containing the control plasmid, during somatic embryo maturation and regeneration, TO plants are analyzed by qPCR. Using the control plasmid in the Agrobacterium, it is expected that the frequency of recovering TO plants (relative to the number of Agrobacterium-infected immature embryos) that contain a modified endogenous ALS locus in addition to the integrated T-DNA (with CAS9, WUS and gRNA expression cassettes at a 5-10% frequency. When the second treatment with the Agrobacterium is tested, it is expected that transformation frequencies will be higher than the control, the growth of transgenic somatic embryos will be accelerated and the frequency of recovering TO plants that TO plants containing the modified ALS gene with no additional integrations of the second T-DNA will be recovered at a higher frequency. It is expected that positioning the dCAS9, REPA and the WUS2 expression cassettes in the second T-DNA (within the lower titer Agrobacterium will provide an improved stimulation of ALs gene editing without the concomitant integration of the morphogenic gene expression cassettes.

### Example 8: Use of a Morphogenic Factor Expression Cassette and a Constitutively-Expressed dCas-Repressor Fusion Cassette Outside the T-DNA Borders Stimulates Transformation and Selection of Single-Copy TO Maize Plants that Contain No Morphogenic Factor or RepA Expression Cassettes

A “Dead-CAS9” (dCAS9) as used herein, is used to supply a transcriptional repressor domain. The dCAS9 has been mutated so that can no longer cut DNA. The dCAS0 can still bind when guided to a sequence by the gRNA and can also be fused to repressor elements (see Gilbert et al., Cell 2013 Jul. 18; 154(2): 442-451, Kiani et al., 2015 November Nature Methods Vol. 12 No. 11: 1051-1054). The dCAS9 fused to the repressor element, as described herein, is abbreviated to dCAS9-REP, where the repressor element (REP) can be any of the known repressor motifs that have been characterized in plants (see Kagale and Rozxadowski, 20010 Plant Signaling & Behavior5:6, 691-694 for review). An expressed guide RNA (gRNA) binds to the dCAS9-REP protein and targets the binding of the dCAS9-REP fusion protein to a specific predetermined nucleotide sequence within a promoter (a promoter within the T-DNA). For example, if this is expressed Beyond—the Border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the Beyond-the-Border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9-REP (Urritia et al., 2003, Genome Biol. 4:231) as described above.

For these experiments, two plasmids are compared. The first contains a T-DNA comprising the following components: RB+SI-UBI3 PRO::ZS-GREEN1::PINII TERM+SB-ALS PRO::ZM-HRA::SB-PEPC1 TERM+LB (the Control Plasmid). The second plasmid contains the same components within the T-DNA (RB+SI-UBI3 PRO::ZS-GREEN1::PINII TERM+SB-ALS PRO::ZM-HRA::SB-PEPC1 TERM+LB) plus three additional expression cassette immediately beyond the left border: i) an expression cassette containing ZM-U6 PRO::gRNA::ZM-U6 TERM; ii) ZM-UBI PRO::dCAS9˜REP::PINII TERM expression cassette; and iii) a PLTP PRO::ZM-WUS2::IN2-1 TERM expression cassette (the Test Plasmid). The gRNA within the first expression cassette has been designed to target the dCAS9-REP to the SB-ALS PRO sequence which transcriptionally silences HRA expression and renders the cell sensitive to imazapyr.

After Agrobacterium-mediated transformation with Agrobacterium strain LBA4404 THY—containing the control plasmid, during somatic embryo maturation and regeneration, TO plants are analyzed by qPCR. Using the control plasmid in the Agrobacterium, it is expected that the frequency of recovering TO plants (relative to the number of Agrobacterium-infected immature embryos) that are single copy for the Trait genes within the T-DNA with no vector (plasmid) backbone will be approximately 5%. When the Test plasmid is used in the Agrobacterium, it is expected that transformation frequencies will be higher than the control, the growth of transgenic somatic embryos will be accelerated and the frequency of recovering TO plants that are single copy for the Trait genes within the T-DNA while lacking integrated WUS and dCAS9-REP and no vector (plasmid) backbone will be increased relative to the control treatment. It is expected that positioning the dCAS9-REP and the WUS2 expression cassettes outside the T-DNA Left Border will provide an improved stimulation of transformation without the concomitant integration of these morphogenic gene expression cassettes.

Alternatively, a synthetic Zinc-Finger Nuclease fused to a repressor domain can be used in place of the gRNA and dCAS9-REP (Urritia et al., 2003, Genome Biol. 4:231) in the above example.

We claim:

1. A method of editing a target polynucleotide in the genome of a target cell, comprising:
(a) providing a Cas endonuclease to the target cell,
(b) providing at least one heterologous morphogenic factor to a non-target cell, wherein the heterologous morphogenic factor is not provided to the target cell, and
(c) incubating the target cell to allow for the Cas endonuclease and a guide RNA to form a complex that recognizes, binds to, and nicks or cleaves the target polynucleotide;
wherein the editing is selected from the group consisting of: insertion of at least one polynucleotide, deletion of at least one polynucleotide, molecular alteration of at least one polynucleotide, substitution of at least one polynucleotide, and a combination of at least two of the preceding.

2. The method of claim 1, wherein the composition of (a) is provided as a polynucleotide on a different construct than the composition of (b).

3. The method of claim 1, wherein the composition of (a) is provided as a polynucleotide on the same construct as the composition of (b).

4. The method of claim 1, wherein the morphogenic factor is provided as a polypeptide.

5. The method of claim 1, wherein the morphogenic factor is provided via upregulation of an endogenous morphogenic factor gene.

6. The method of claim 1, wherein a polynucleotide sequence encoding the Cas endonuclease or the guide RNA or both is provided as a recombinant construct on a T-DNA vector between the left and right borders, wherein the T-DNA vector further comprises the at least one morphogenic factor outside of the left and right borders.

7. The method of claim 1, wherein either the Cas endonuclease of (a) or the morphogenic factor of (b) or both further comprise(s) a cell penetrating peptide motif at the 3′ end, the 5′ end, or both ends.

8. The method of claim 1, wherein the Cas endonuclease of (a) is provided as a polynucleotide molecule on a first vector that further comprises, outside of the T-DNA borders, a repressor fused to a polynucleotide encoding a second Cas endonuclease that lacks nuclease activity, wherein said repressor is capable of binding to a regulatory element operably linked to the polynucleotide encoding the Cas endonuclease of (a).

9. A method of editing a polynucleotide in the genome of a target cell, comprising:
(a) providing a Cas endonuclease to a cell, and
(b) providing at least one morphogenic factor to the cell, wherein the morphogenic factor is provided as a recombinant construct as part of a T-DNA vector, wherein the polynucleotide sequence encoding the morphogenic factor is outside of the left and right T-DNA borders; and
(c) incubating the target cell to allow for the Cas endonuclease and a guide RNA to form a complex that recognizes, binds to, and nicks or cleaves the target polynucleotide;
wherein the editing is selected from the group consisting of: insertion of at least one polynucleotide, deletion of at least one polynucleotide, molecular alteration of at least one polynucleotide, substitution of at least one polynucleotide, and a combination of at least two of the preceding.

10. The method of claim 9, wherein the composition of (a) is provided as a polynucleotide on a different construct than the composition of (b).

11. The method of claim 9, wherein the composition of (a) is provided as a polynucleotide on the same construct as the composition of (b).

12. The method of claim 9, wherein the morphogenic factor is provided as a polypeptide.

13. The method of claim 9, wherein either the Cas endonuclease of (a) or the morphogenic factor of (b) or both further comprise(s) a cell penetrating peptide motif at the 3′ end, the 5′ end, or both ends.

14. The method of claim 9, wherein the Cas endonuclease of (a) is provided as a polynucleotide molecule on a first vector that further comprises, outside of the T-DNA borders, a repressor fused to a polynucleotide encoding a second Cas endonuclease that lacks nuclease activity, wherein said repressor is capable of binding to a regulatory element operably linked to the polynucleotide encoding the Cas endonuclease of (a).

15. A method of editing a polynucleotide in the genome of a target cell, comprising:
(a) providing a Cas endonuclease to a cell,
(b) providing at least one morphogenic factor to the cell, wherein the morphogenic factor is provided via stimulation or upregulation of an endogenous morphogenic factor gene, and
(c) incubating the target cell to allow for the Cas endonuclease and a guide RNA to form a complex that recognizes, binds to, and nicks or cleaves the target polynucleotide;
wherein the editing is selected from the group consisting of: insertion of at least one polynucleotide, deletion of at least one polynucleotide, molecular alteration of at least one polynucleotide, substitution of at least one polynucleotide, and a combination of at least two of the preceding.

16. The method of claim 1, claim 9, or claim 15, wherein the morphogenic factor is selected from the group consisting of: Wuschel, Ovule Development Protein, and Babyboom.

17. The method of claim 1, claim 9, or claim 15, further comprising introducing a polynucleotide modification template into the target cell.

18. The method of claim 1, claim 9, or claim 15, further comprising introducing a heterologous polynucleotide donor DNA molecule into said target cell.

19. The method of claim 1, claim 9, or claim 15, wherein the cell is a plant cell, and a benefit is conferred to the plant cell or to a tissue or organism comprising the plant cell; wherein said benefit is selected from the group consisting of: improved health, improved growth, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction.

20. The method of claim 1, claim 9, or claim 15, further comprising regenerating an organism or tissue from the cell.

21. The method of The method of claim 1, claim 9, or claim 15, wherein the editing of a polynucleotide in the genome of the cell modulates a trait of agronomic importance in a plant obtained or derived from the cell, wherein the trait of agronomic importance is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition; as compared to an isoline plant not comprising or derived from a cell whose genome was edited with said RNA-guided Cas endonuclease.


SEQUENCE LISTING (110 sequences)

SEQ ID NO: 1 - DNA, 2040 bp, Zea mays
  source 1..2040: mol_type = genomic DNA
atggcttcagcgaacaactggctgggcttctcgctctcgggccaggataacccgcagcctaaccaggacagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaagagctagaagaaatgaaacatatgactagacaggagtacattgcatacctaagaagaaatagcagtggattttctcgtggggcatcaaagtatcgtggagtaactagacatcatcagcatgggagatggcaagcaaggatagggagagttgcaggaaacaaggatctttacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 2 - DNA, 1680 bp, Oryza sativa
  source 1..1680: mol_type = genomic DNA
atggcctccatcaccaactggctcggcttctcctcctcctccttctccggcgccggcgccgaccccgtcctgccccacccgccgctgcaagagtgggggagcgcttatgagggcggcggcacggtggcggccgccggcggggaggagacggcggcgccgaagctggaggacttcctcggcatgcaggtgcagcaggagacggccgccgcggcggcggggcacggccgtggaggcagctcgtcggtcgttgggctgtccatgatcaagaactggctacgcagccagccgccgcccgcggtggttgggggagaagacgctatgatggcgctcgcggtgtcgacgtcggcgtcgccgccggtggacgcgacggtgccggcctgcatttcgccggatgggatggggtcgaaggcggccgacggcggcggcgcggccgaggcggcggcggcggcggcggcgcagaggatgaaggcggccatggacacgttcgggcagcggacgtccatctaccggggtgtcaccaagcacaggtggacaggaaggtatgaagcccatctttgggataacagctgcagaagagaaggtcagactcgcaaaggcagacaagtcaatgcaggaggatatgataaggaagaaaaagctgctagggcttatgatttggctgcccttaaatactggggcactacaacgacgacgaattttccggtaagcaactacgaaaaagagttggatgaaatgaagcacatgaataggcaggaatttgttgcatcccttagaagaaaaagcagtggattttcacgtggtgcttccatatatcgtggtgttacaagacaccatcagcatggaaggtggcaagcaaggataggacgggtggcaggaaacaaggatctgtatttgggcacatttggcacccaagaggaagctgcagaggcatatgatatcgctgcaatcaaattccgtggtctcaatgctgtgacaaactttgacatgagccggtacgatgtcaagagcatcattgaaagcagcaatctcccaattggtactggaaccacccggcgattgaaggactcctctgatcacactgataatgtcatggacatcaatgtcaataccgaacccaataatgtggtatcatcccacttcaccaatggggttggcaactatggttcgcagcattatggttacaatggatggtcgccaattagcatgcagccgatcccctcgcagtacgccaacggccagcccagggcatggttgaaacaagagcaggacagctctgtggttacagcggcgcagaacctgcacaatctacatcattttagttccttgggctacacccacaacttcttccagcaatctgatgttccagacgtcacaggtttcgttgatgcgccttcgaggtccagtgactcatactccttcaggtacaatggaacaaatggctttcatggtctcccgggtggaatcagctatgctatgccggttgcgacagcggtggaccaaggtcagggcatccatggctatggagaagatggtgtggcaggcattgacaccacacatgacctgtatggcagccgtaatgtgtactacctttccgagggttcgcttcttgccgatgtcgaaaaagaaggcgactatggccaatctgtggggggcaacagctgggttttgccgacaccgtag

SEQ ID NO: 3 - DNA, 2104 bp, Oryza sativa
  source 1..2104: mol_type = genomic DNA
atggcttctgcaaacaactggctgggcttctcgctctccggccaagagaatccgcagcctcaccaggatagctcgcctccggcagccatcgacgtctccggcgccggcgacttctatggcctgccgacgtcgcagccgacggcggccgacgcgcacctcggcgtggcggggcatcatcacaacgcctcgtatggcatcatggaggccttcaataggggagctcaagaggcacaagattggaacatgagggggctggactacaacggcggcgcctcggagctgtcgatgctcgtcggctccagcggcggcaagagggcggcggcggtggaggagaccgagccgaagctggaggacttcctcggcggcaactcgttcgtctccgagcaagatcatcacgcggcggggggcttcctcttctccggcgtcccgatggccagcagcaccaacagcaacagcgggagcaacactatggagctctccatgatcaagacctggctccggaacaacggccaggtgcccgccggccaccagccgcagcagcagcagccggcggccgcggccgccgccgcgcagcagcaggcgcacgaggcggcggagatgagcaccgacgcgagcgcgagcagcttcgggtgctcctccgacgcgatggggaggagtaacaacggcggcgcggtctcggcggcggccggcgggacgagctcgcagagcctggcgctctcgatgagcacgggctcgcactcgcacctgcctatcgtcgtcgccggcggcgggaacgccagcggcggagcggccgagagcacatcgtcggagaacaagcgggccagcggcgccatggattcgccgggcggtggcgcgatagaggccgtgccgaggaagtccatcgacacgttcgggcaaaggacctcgatatatcgaggtgtaacaaggcatagatggacagggcgatatgaggctcatctctgggataatagctgtagaagagaagggcagagtcgcaagggtaggcaaggtggctatgacaaggaggataaagcagcgagagcttatgatttggcagctctgaagtattggggcacaacaacaacaacaaatttcccaataagtaactatgaaaaagagctagatgaaatgaaacatatgaccaggcaggagtatattgcatacctaagaaggaatagcagtggattttctcgtggtgcatcgaaatatcgtggtgtaaccaggcaccatcagcatgggagatggcaagcaaggatagggagggttgcaggaaacaaggacctctacttaggcaccttcagcaccgaggaggaggcggcggaggcgtacgacatcgcggcgatcaagttccgggggctcaacgccgtcaccaactttgacatgagccgctacgacgtcaagagcatcctggagagcagcacgctgccggtgggcggcgcggcgaggcggctgaaggaggcggcggaccacgcggaggcggccggcgccaccatctggcgcgccgccgacatggacggcgccggcgtcatctccggcctggccgacgtcgggatgggcgcctacgccgcctcgtaccaccaccaccaccaccacggctggccgaccatcgcgttccagcagccgccgccgctcgccgtgcactacccgtacggccaggcgccggcggcgccgtcgcgcgggtggtgcaagcccgagcaggacgccgccgtcgctgccgccgcgcacagcctccaggacctccagcagctgcacctcggcagcgccgccgcccacaacttcttccaggcgtcgtcgagctcgacggtctacaacggcggcggcggcgggtaccagggcctcggtggcaacgccttcttgatgccggcgagcaccgtcgtggccgaccaggggcacagcagcacggccaccaaccatggaaacacctgcagctacggcaacgaggagcaggggaagctcatcgggtacgacgccatggcgatggcgagcggcgccgccggcggcgggtaccagctgtcgcagggctcggcgtcgacggtgagcatcgcgagggcgaacggctactcggccaactggagctcgcctttcaatggcgccatgggatgat

SEQ ID NO: 4 - DNA, 1977 bp, Oryza sativa
  source 1..1977: mol_type = genomic DNA
atggcttctgcagataactggctaggcttctcgctctccggccaaggcaacccacagcatcaccagaacggctcgccgtctgccgccggcgacgccgccatcgacatctccggctcaggcgacttctatggtctgccaacgccggacgcacaccacatcggcatggcgggcgaagacgcgccctatggcgtcatggatgctttcaacagaggcacccatgaaacccaagattgggcgatgaggggtttggactacggcggcggctcctccgacctctcgatgctcgtcggctcgagcggcggcgggaggaggacggtggccggcgacggcgtcggcgaggcgccgaagctggagaacttcctcgacggcaactcattctccgacgtgcacggccaagccgccggcgggtacctctactccggaagcgctgtcggcggcgccggtggttacagtaacggcggatgcggcggcggaaccatagagctgtccatgatcaagacgtggctccggagcaaccagtcgcagcagcagccatcgccgccgcagcacgctgatcagggcatgagcaccgacgccagcgcgagcagctacgcgtgctccgacgtgctggtggggagctgcggcggcggcggcgccgggggcacggcgagctcgcatgggcagggcctggcgctgtcgatgagcacggggtcggtggccgccgccggagggggcggcgccgtcgtcgcggccgagagctcgtcgtcggagaacaagcgggtggattcgccgggcggcgccgtggacggcgccgtcccgaggaaatccatcgacaccttcgggcaaaggacgtctatataccgaggtgtaacaaggcatagatggacaggaagatatgaagctcatctgtgggataatagctgtaggagagaaggccaaagtcgcaaggggagacaggtttgtgtaggcggttatgacaaagaagataaggcggctcgggcttatgatttggcagctctaaaatactggggcacgaccacaacaacaaatttcccaatgagtaattatgaaaaggagctagaggaaatgaaacacatgaccaggcaggagtacattgcacatcttagaaggaatagcagtggattttctcgtggtgcatccaaatatcgtggtgttactaggcatcatcagcatgggagatggcaggcaaggatagggcgagttgcaggcaacaaggatatctacctaggcaccttcagcaccgaggaggaggccgccgaggcgtacgacatcgccgccatcaagttccgcgggctcaacgccgtcaccaacttcgacatgagccggtacgacgtcaagagcatcctggacagcagcacgctgccggtcggcggcgcggcgcggcggctcaaggaggcggaggtcgccgccgccgccgcgggcggcggcgtgatcgtctcccacctggccgacggcggtgtgggtgggtactactacgggtgcggcccgaccatcgcgttcggcggcggcggccagcagccggcgccgctcgccgtgcactacccgtcgtacggccaggccagcgggtggtgcaagccggagcaggacgcggtgatcgcggccgggcactgcgcgacggacctccagcacctgcacctcgggagcggcggcgccgccgccacccacaacttcttccagcagccggcgtcaagctcggccgtctacggcaacggcggcggcggcggcggcaacgcgttcatgatgccgatgggcgccgtggtggccgccgccgatcacggcgggcagagcagcgcctacggcggtggcgacgagagcgggaggctcgtcgtggggtacgacggcgtcgtcgacccgtacgcggccatgagaagcgcgtacgagctctcgcagggctcgtcgtcgtcgtcggtgagcgtcgcgaaggcggcgaacgggtacccggacaactggagctcgccgttcaacggcatgggatga

SEQ ID NO: 5 - DNA, 2082 bp, Sorghum bicolor
  source 1..2082: mol_type = genomic DNA
atggcttcgacgaacaaccactggctgggtttctcgctctcgggccaggataacccgcagcctaatcatcaggacagctcgcctgccgccgccggcatcgacatctccggcgccagcgacttctatggcttgcccacgcagcagggctccgacgggaatctcggcgtgccgggcctgcgggacgatcacgcttcttatggcatcatggaggccttcaacagggttcctcaagaaacccaagattggaacatgaggggattggactacaacggcggtggctcggaactctcgatgcttgtggggtccagcggcggcggcgggggcggcggcaagagggccgtggaagacagcgagcccaagctcgaagatttcctcggcggcaactcgttcgtctccgagcatgatcagtccggcggttacctgttctctggagtcccgatggccagcagcaccaacagcaacagcgggagcaacaccatggagctctccatgatcaagacctggctccggaacaaccaggtgccccagccgcagccgccagcagctccgcatcaggcgccgcagactgaggagatgagcaccgacgccaacgccagcgccagcagctttggctgctcggattcgatggggaggaacggcacggtggcggctgctgggagctcccagagcctggcgctctcgatgagcacgggctcgcacctgccgatggttgtggccggcggcggcgccagcggagcggcctcggagagcacgtcatcggagaacaagcgagcgagcggcgccatggattcgcccggcagcgcggtagaagccgtcccgaggaagtccatcgacacgttcgggcaaaggacctctatatatcgaggtgtaacaagacatagatggacagggcgatatgaggctcatctatgggataatagttgtagaagagaagggcagagtcgcaagggtaggcaagtttaccttggtggctatgacaaggaagacaaggcagcaagggcttatgatttggcagctctcaagtattggggcactactacaacaacaaatttccctataagcaactatgaaaaggagctagaggaaatgaaacatatgactaggcaggagtatattgcatacctaagaagaaatagcagtggattttctcgtggcgcatcaaaatatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctgaacgccgtcaccaacttcgacatgagccgctacgacgtcaagagcatcctcgagagcagcacgctgcctgtcggcggcgcggccaggcgcctcaaggatgccgtggaccacgtggaggccggcgccaccatctggcgcgccgacatggacggcggcgtgatctcccagctcgccgaagccgggatgggcggctacgcctcgtacgggcaccacgcctggccgaccatcgcgttccagcagccgtcgccgctctccgtccactacccgtacgggcagccgccgtcccgcgggtggtgcaagcccgagcaggacgcggccgtcgccgccgccgcgcacagcctgcaggacctccagcagctgcacctcggcagcgcggcacacaacttcttccaggcgtcgtcgagctcggcagtctacaacagcggcggcggcggcgctagcggcgggtaccaccagggcctcggtggcggcagcagctccttcctcatgccgtcgagcactgtcgtggcgggggccgaccaggggcacagcagcagcacggccaaccaggggagcacgtgcagctacggggacgatcaccaggaagggaagctcatcgggtacgacgccatggtggcggcgaccgcagccggcggggacccgtacgccgcggcgaggagcgggtaccagttctcgtcgcagggctcgggatccacggtgagcatcgcgagggcgaacgggtactctaacaactggagctctcctttcaacggcggcatggggtga

SEQ ID NO: 6 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 7 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVNAGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 8 - protein, 700 aa, Oryza sativa
MASANNWLGFSLSGQENPQPHQDSSPPAAIDVSGAGDFYGLPTSQPTAADAHLGVAGHHHNASYGIMEAFNRGAQEAQDWNMRGLDYNGGASELSMLVGSSGGKRAAAVEETEPKLEDFLGGNSFVSEQDHHAAGGFLFSGVPMASSTNSNSGSNTMELSMIKTWLRNNGQVPAGHQPQQQQPAAAAAAAQQQAHEAAEMSTDASASSFGCSSDAMGRSNNGGAVSAAAGGTSSQSLALSMSTGSHSHLPIVVAGGGNASGGAAESTSSENKRASGAMDSPGGGAIEAVPRKSIDTFGQRTSIYRGVTRHRWTGRYEAHLWDNSCRREGQSRKGRQGGYDKEDKAARAYDLAALKYWGTTTTTNFPISNYEKELDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKEAADHAEAAGATIWRAADMDGAGVISGLADVGMGAYAASYHHHHHHGWPTIAFQQPPPLAVHYPYGQAPAAPSRGWCKPEQDAAVAAAAHSLQDLQQLHLGSAAAHNFFQASSSSTVYNGGGGGYQGLGGNAFLMPASTVVADQGHSSTATNHGNTCSYGNEEQGKLIGYDAMAMASGAAGGGYQLSQGSASTVSIARANGYSANWSSPFNGAMG

SEQ ID NO: 9 - protein, 658 aa, Oryza sativa
MASADNWLGFSLSGQGNPQHHQNGSPSAAGDAAIDISGSGDFYGLPTPDAHHIGMAGEDAPYGVMDAFNRGTHETQDWAMRGLDYGGGSSDLSMLVGSSGGGRRTVAGDGVGEAPKLENFLDGNSFSDVHGQAAGGYLYSGSAVGGAGGYSNGGCGGGTIELSMIKTWLRSNQSQQQPSPPQHADQGMSTDASASSYACSDVLVGSCGGGGAGGTASSHGQGLALSMSTGSVAAAGGGGAVVAAESSSSENKRVDSPGGAVDGAVPRKSIDTFGQRTSIYRGVTRHRWTGRYEAHLWDNSCRREGQSRKGRQVYLGGYDKEDKAARAYDLAALKYWGTTTTTNFPMSNYEKELEEMKHMTRQEYIAHLRRNSSGFSRGASKYRGVTRHHQHGRWQARIGRVAGNKDIYLGTFSTEEEAAEAYDIAAIKFRGLNAVTNFDMSRYDVKSILDSSTLPVGGAARRLKEAEVAAAAAGGGVIVSHLADGGVGGYYYGCGPTIAFGGGGQQPAPLAVHYPSYGQASGWCKPEQDAVIAAGHCATDLQHLHLGSGGAAATHNFFQQPASSSAVYGNGGGGGGNAFMMPMGAVVAAADHGGQSSAYGGGDESGRLVVGYDGVVDPYAAMRSAYELSQGSSSSSVSVAKAANGYPDNWSSPFNGMG

SEQ ID NO: 10 - protein, 693 aa, Sorghum bicolor
MASTNNHWLGFSLSGQDNPQPNHQDSSPAAAGIDISGASDFYGLPTQQGSDGNLGVPGLRDDHASYGIMEAFNRVPQETQDWNMRGLDYNGGGSELSMLVGSSGGGGGGGKRAVEDSEPKLEDFLGGNSFVSEHDQSGGYLFSGVPMASSTNSNSGSNTMELSMIKTWLRNNQVPQPQPPAAPHQAPQTEEMSTDANASASSFGCSDSMGRNGTVAAAGSSQSLALSMSTGSHLPMVVAGGGASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGGVISQLAEAGMGGYASYGHHAWPTIAFQQPSPLSVHYPYGQPPSRGWCKPEQDAAVAAAAHSLQDLQQLHLGSAAHNFFQASSSSAVYNSGGGGASGGYHQGLGGGSSSFLMPSSTVVAGADQGHSSSTANQGSTCSYGDDHQEGKLIGYDAMVAATAAGGDPYAAARSGYQFSSQGSGSTVSIARANGYSNNWSSPFNGGMG

SEQ ID NO: 11 - DNA, 2130 bp, Zea mays
  source 1..2130: mol_type = genomic DNA
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 12 - DNA, 2133 bp, Zea mays
  source 1..2133: mol_type = genomic DNA
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 13 - DNA, 2112 bp, Sorghum bicolor
  source 1..2112: mol_type = genomic DNA
atggctactgtgaacaactggctcgctttctccctctccccgcaggagctgccgcccacccagacggactccaccctcatctctgccgccaccaccgacgatgtctccggcgatgtctgcttcaacatcccccaagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggcatagatggactgggagatatgaagcacatctgtgggacaacagttgcagaagggaaggacaaactcgcaagggtcgtcaagtctatttaggtggctatgataaagaggagaaagctgctagggcttatgatctggctgctcttaagtactggggtcccacgacaacaacaaattttccagtgaataactacgaaaaggagctggaggatatgaagcacatgacaaggcaggagtttgtagcgtctctgagaaggaagagcagtggtttctccagaggtgcatccatttacaggggagtgactaggcatcaccagcatggaagatggcaagcacggattggacgagttgcagggaacaaggatctctacttgggcaccttcagcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatacttaa

SEQ ID NO: 14 - DNA, 2112 bp, Setaria italica; identical to SEQ ID NO: 13
  source 1..2112: mol_type = genomic DNA

SEQ ID NO: 15 - DNA, 2064 bp, Brachypodium distachyum
  source 1..2064: mol_type = genomic DNA
atggctaccgtgaacaactggctgggcttctccctctccccgcaggagctcccgtcctccgccgctgccgccgccggcgacgtctccggcgccgacgtctgcttcaacatcccgcaagattggggcatgaggggatcggagctgtcggcgctggtggcggagccgaagctggaggacttcctcggcggcatctcttcctactctgaccatcacaaggcagccagaagcaacaacatgaacatcaatggcgccgccgcctgctacgcgagctccggcagcagcggctaccagctttaccacgaccaccccaactccctccagttcgccgactctgtcatggtcgcttcctccgctggcggcgtccacaatgaacatggcatcatggcaagcaccactgccaatggtgcaggcaccaatggcggcatcgggctgtccatgatcaagagctggctgcggagccagccggcgccggcgcagcaggagcagcagagggcggaggggctgtcgctgtccatgaacatgccgctcctgcaggcggcggcggcggagactagcttgtccacgtcggcagcgcagcagggatctcggaaggagaataatggcagcagtagtgctggttctggagccgtggtgtcagccggtactaccagcgccggcgcggtggtggtggagtcgccggcagcggggaggaagaccgccgacacgttcgggcagcggacttcgatctaccgcggcgtcaccaggcatagatggacagggaggtatgaggctcacctgtgggacaacagctgcagaagagaaggacaaactcgcaagggtcgtcaagtctatctcggtggttatgacaaagaggagaaagctgcaagagcttatgatttggctgctctcaagtattggggccccaccacgacgacaaattttccagtagataactacgagaaggagctggaggagatgaagcacatgacaaggcaggagtttgtggcgtctctccgaaggaagagcagtggtttttcaagaggtgcatccatttatcgtggagtaactaggcaccaccaacatgggagatggcaagcaaggatagggagagttgcagggaacaaggatctctacttgggcactttcagcacgcaggaggaggcggcggaggcgtacgacatcgcggcgatcaagttccgggggctgaacgccgtcactaacttcgacatgtcccgctacgacgtcaagagcatcctcgacagcaccgccgcgctgcctgtcggcggcaccaagcgcctcagggacgcggctgccgccgatcagcactaccagcagcgcgccgggggcgtcgtcagctacgccgcgccgcagctcggtggtgtcaacgagaccgcccttgcctatggcgcgccctactaccaccaccaaacctccgccgcagcgtggccgaccatcgcgttccaggcggcgccgcaagcgtcgtcggggcacgggcacatgctgtaccacccgtacgggcagccattgatgcgcgggtggtgcaagcaagagcaggagcaagggcaagggcagcaggagccggaccacgcggtgatcgcggccgcgcacagcctgcaggacctccaccacctcaacctcggcgccggtgcgcacgacttcttctcccagcatgcccacgccatgcaccagcagcagcagcagcacggcggcctcggcagcgtcgacaacaacggcgcggcgtcgctggagcacagcactggctccaactccgtcgtctacaacggggccgctgccgccggggacaccaacaacagctacatgctgccgcccatgagcgctgcagcagcagctggcttcggcctccgcgatcagcaggacgaaggcgggaagatggcgtacgagaacttcctcctcggcgccgccaccgacggctactgtggccctggaaggatggcggccacctggacaccggtgtcggtttcggcggcccagccagtggcggcaacgagcagcggcagcgacatggccggtgccgtctgccatggcggcgcgcagctcttcagcgtctggaacgacgacagttag

SEQ ID NO: 16 - DNA, 3769 bp, Sorghum bicolor
  source 1..3769: mol_type = genomic DNA
atggctactgtgaacaactggctcgctttctccctctccccgcaggagctgccgcccacccagacggactccaccctcatctctgccgccaccaccgacgatgtctccggcgatgtctgcttcaacatcccccaaggtatgcatctatcgatcgatatatgtacgtacagtgcgcatatatatatatatctgcagtttgtggtacgaatactgattgaagctagcatgaaatgtcgtttgttctttcagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggtaataagggtccggtattacaatgaatcgtcacttcgtcagagaactaaactagcacaaatcagcaatgaatcaagtaatatcatgaaatttagaaaagccgttagcaatgcaaggagctatcattatagatttgattgcatctagacagttctgaattaaatgagtagggcaatgtgtagcctttgatgatctcgctgattattaggagtgccatttgtattggctatgattgtggtatatacagcagtagacaattaacaaaaggctaccactttcgaattattttaggcatagatggactgggagatatgaagcacatctgtgggacaacagttgcagaagggaaggacaaactcgcaagggtcgtcaaggtaccaatataatgcaatacaccgtatttaaatatatatgcttttctgtaattaagtttatactttcacaaaactgacattacttcgcattatcatttttggattgtcgtcgtcatgattggcgggattgaaatgaactattgaatctacagtctatttaggtaagcgatttcacttggttattaatttgggaccaactacttaatccagtttgtttttcccctataaccattattttttcatctgtgttctcaactcttacttttccatcttgttccactgataggtggctatgataaagaggagaaagctgctagggcttatgatctggctgctcttaagtactggggtcccacgacaacaacaaattttccagtatgtatatgtagaatgcagttttacttcactgaagatcatacctttgctatgtctcaaatgccgttcattagttagtggatctgaagtgaaggttctgtaatttttgttaactatgtacattgctggaattgtacttaaagtcatttgtttttgtatatctaggtgaataactacgaaaaggagctggaggatatgaagcacatgacaaggcaggagtttgtagcgtctctgagaaggtcggtcgaacagcattgattaatcaatgccaactctattgaataaacatctactctgttaattgttaaagtttgagagaaagatctgcatgttagatcttaatagaccactgtatatgaatgcaggaagagcagtggtttctccagaggtgcatccatttacaggggagtgactaggtatgaattcatataatggcgtcaacaaacacacatacactttgattgaggaggcgaatgcacgcatggattgaatgtgaatggtgttttacttgaactatgtaattataggcatcaccagcatggaagatggcaagcacggattggacgagttgcagggaacaaggatctctacttgggcaccttcagtaagtatcagagatgttttctcattgtatatagaggagtacttctatatgtatatatacattcagttattcaccacacaaaagcaaattgcagtcaactaataacaatctcaacgcaatgagaagcaagtgttacagctgatagtacacatttgtagaccttctgcatatggatgttatatatgatgactattaaaaatgtgaccattgcatcaagtcatgcaaagttgcattgcagtagtacatacattacttagtgcatgctcctcaagtggctttttcaaacctgatcccatgtctggcgctattgttgtctcccattcacccgtgcatcaggtcaaaatagtactatgcctcaataagaaacacatgagcatgcactggcagcagcagactaatcaagttctatcatttactaataaactaattaggctacagcatccaaaagattctacccattaagccacaactgttcatgcatgcattcataaaccaggataccaccatgcatgcgtgcaccgtgttcgtgcttggaatattgagctgagccgagtgcacccttgcgtggatgcaggcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatacttaa

SEQ ID NO: 17 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGNGGAQLFSVWNDT

SEQ ID NO: 18 - protein, 709 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 19 - protein, 703 aa, Sorghum bicolor
MATVNNWLAFSLSPQELPPTQTDSTLISAATTDDVSGDVCFNIPQDWSMRGSELSALVAEPKLEDFLGGISFSEQHHKANCNMIPSTSSTACYASSGATAGYHHQLYHQPTSSALHFADSVMVASSAGGVHDGGAMLSAASANGSAGAGAASANGSGSIGLSMIKNWLRSQPAPMQPRVAAAESVQGLSLSMNMAGATQGAAGMPLLAGERGRAPESVSTSAQGGAVVTAPKEDSGGSGVAATGALVAVSTDTGGSGASADNTARKTVDTFGQRTSIYRGVTRHRWTGRYEAHLWDNSCRREGQTRKGRQVYLGGYDKEEKAARAYDLAALKYWGPTTTTNFPVNNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVVSYDVGRIASQLGDGGALAAAYGAHYHGAWPTIAFQPSAATGLYHPYAQPMRGWCKQEQDHAVIAAAHSLQELHHLNLGAAAGAHDFFSAGQQAAMHGLGSMDNASLEHSTGSNSVVYNGVGDSNGSTVVGSGGYMMPMSAATATATTAMVSHEQVHARAQGDHHDEAKQAAQMGYESYLVNAENYGGGRMSAAWATVSAPPAASSNDNMADVGHGGAQLFSVWNDT

SEQ ID NO: 20 - protein, 703 aa, Setaria italica; identical to SEQ ID NO: 19

SEQ ID NO: 21 - protein, 687 aa, Brachypodium distachyum
MATVNNWLGFSLSPQELPSSAAAAAGDVSGADVCFNIPQDWGMRGSELSALVAEPKLEDFLGGISSYSDHHKAARSNNMNINGAAACYASSGSSGYQLYHDHPNSLQFADSVMVASSAGGVHNEHGIMASTTANGAGTNGGIGLSMIKSWLRSQPAPAQQEQQRAEGLSLSMNMPLLQAAAAETSLSTSAAQQGSRKENNGSSSAGSGAVVSAGTTSAGAVVVESPAAGRKTADTFGQRTSIYRGVTRHRWTGRYEAHLWDNSCRREGQTRKGRQVYLGGYDKEEKAARAYDLAALKYWGPTTTTNFPVDNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSTAALPVGGTKRLRDAAAADQHYQQRAGGVVSYAAPQLGGVNETALAYGAPYYHHQTSAAAWPTIAFQAAPQASSGHGHMLYHPYGQPLMRGWCKQEQEQGQGQQEPDHAVIAAAHSLQDLHHLNLGAGAHDFFSQHAHAMHQQQQQHGGLGSVDNNGAASLEHSTGSNSVVYNGAAAAGDTNNSYMLPPMSAAAAAGFGLRDQQDEGGKMAYENFLLGAATDGYCGPGRMAATWTPVSVSAAQPVAATSSGSDMAGAVCHGGAQLFSVWNDDS

SEQ ID NO: 22 - DNA, 2133 bp, Zea mays
  source 1..2133: mol_type = genomic DNA
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggaatggcggcgcgcagctcttcagtgtctggaacgacacttaa

SEQ ID NO: 23 - DNA, 909 bp, Zea mays
  source 1..909: mol_type = genomic DNA
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 24 - DNA, 666 bp, Zea mays
  source 1..666: mol_type = genomic DNA
tcaggcgcggcgcctccctcgcccaccctcggcttctacgccgccggcaatggcggcggatcggctgtgctgctggacacgagttccgactggggcagcagcggcgctgccatggccaccgagacatgcttcctgcaggtcggtgctgtagtacgttcttttcttgggcattgcgcgcagtttcacgttcgtacgtacgagttgatcgccgcgtcgttccatccaccggtatatataactgttaggtacggcggtgcgcgcccgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 25 - DNA, 3629 bp, Zea mays
  source 1..3629: mol_type = genomic DNA
gcgggattgccgcgggggagggagattgtcgcggtgacgccgagattgccgctggggcgagcgccgagattgccgggggagggcgaggcgggtgggatgcggggtgccccacggcagaaccgtgcaccactgtcgggctgtgaacgtctacaatacttacttatgtagtagtagatatatatatatatatatatataatgtaaaacaatattttaattataatgtaaacagtgttttacatgattatatacaagatcgactagatagagtttaaggccatatctagcggaacaggtatataagcgtgcaaaacaatgttttgttttgtacgctacactgtttttagagtggagttaactaaaaccttaacttttttttgcaaataaatctctaaataatgtcgtactgtatacactcgagatttccatcgcacaagacacgaaaaaatcccgatcaatttaacgaacattgttttgcattatagattatattgtttacagaatgaagttaactaaaaccttaaccttttgcagataaatctctaaatagtgccgtactgtatacactcgagatttccaccgcacaagacatgagaaaattccggtcgatttgacaaagactgggtgttattaattagaggaagcagatccagccacatgttgtctcacatctgatcccccacgtatagtcgtatacgtttggcccaaacctagctcgatccatgtatgaaacacgtctcgtctcgccttctacctcctttttctatcacaggagattaaagtgagagagagagggcgctcaatgaactgcggcattgaacaatggagctgcaagagcaatgatgcactagctagtgtaatgcagtgcatgcatggtagattggtagcttgcctttgcagtttgcaccaggcaccagcagcagctagaagacgacagacgacaggggtttggctgctaggttgcggaagggcagttaccagttgccacaaggggagcctggccctctgcatcctcctcatgatagctctgtctctctctctcacagacacacacacagagactcttccaaattccgaagcggccaatgcaatgcaagagccagcccccggccgtgtgtcaacttcacttgtctctctccaaaagatatcgtatcacccatggccatgacccccctcccccagccccaacctatatcacctagcgcagctacgctctcttctcccgctctcgctctctgcatgctagctaccttctagctatctagcctctaggtccaatgcactccctccttataaacaaggaaccctccttcgcctctcttgccatagaccggacaccggagaggtcactgcacaggagcgctcaggaaggccgctgcgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggtatgtgcgtcagtgcgtgtggtgtgggtagtatatatatatatatatatatatatatatatatatatatatatatatatatatatatatatatggcctctctttgcattggcacgccaatcggccatcgagatccaatcatatatatcatccaaacgtatagtattagtacatctgtacatgtgactgcaaactgacgtgcacgcgtgtgcaccgtcgtcatcactgatcaggcgcggcgcctccctcgcccaccctcggcttctacgccgccggcaatggcggcggatcggctgggctgctggacacgagttccgactggggcagcagcggcgctgccatggccaccgagacatgcttcctgcaggtgctgtagtacgttcttttcttggggattgcgcgcagtttcacgtacgtacgtacgttcgagtcgatcgccgcgtcgttccatccaccggccggtatatataactgttgggtacggcggtgcgcgcccgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtgagagcaacgcgagctaccactgggacgtgcgttgctgtcattgtcctaggttagtagctagtgccagttactagtaagcatcaggcataggagtatgtagtagaagcatgtctggagaaaggcaatagctagcgtttgggagatctctggcggtactattattagatagcgaatttgcatactatgcagcatgcatgttgccggccgggcgggctttagactccagctactgcatgcgtgcatgcggtggtcctcatgtatgtcggcatgtgtgcgtgtgtgtgtggcagcagcagcagcagtgaattctagtgcagtggtggtggttgtaacgtgtagtttgggggcaggggcacagttgtgtcgaacttgtgcggttgtcccttccttttgtgacttccagaaaagggaatgaggcctgcctgcgtgtctgcttttccatgctcacctcgcctgttgtctgttgagacttcactcagccctgttggtgccttgtagttccagttccagagcatatgcaggtgcaggaatggcatctctaccactgctttcgacttgttggcttgttttcttctatgatactagtgtacttggattgaatgatcacattttttgctggcctcgtatctttgaaatgatcatgttaatttggttacacataagacgataagggtaggtagttcaatgctattagaaaactaggtatttttagttatattttaatccaggaaaaaatatataaaaactaatcgtgatatatagaaatacgcatatgcaaacttagtataataattatgaatacaatagatagtaacctgattgtatatacttttaagggaaaatagatatggcaatgggcctcgatcttcgattctctgcggagaattcttccat

SEQ ID NO: 26 - DNA, 3313 bp, Zea mays
  source 1..3313: mol_type = genomic DNA
aataatggtgtacactcgagatttccaccgcacaagacatgaaaaaatcccggtaaatttgacaaacacttttttgcattgtaaactacaatatttatagagcggagttaactaaaacctcaactttttgcaaataaatctctaaatagtgcgggactgtatacactcgagatttccaccgcacaagacatgagaaaattccggtcaatttgacaaagactggatgttattaatcagaggaagcagatcaagccacatgttgtctcacatctgatcccccacgtattatacgtttggcccaaacctagctcgatccatgtattaaacacgtctcgtttcgccttctacctcctttttctatcacaggagattaaagtgagagagagagggcgctcaatgaactgcggcattgaacaatggagctgcaagagcaatgatgcactagctagtataatgcagaagcaatgcatggtagattggtagctagcctttgcagtttgcaccaggcaccagcagcagctagaagacgacagacgacaggggcttgactaggttgcggaagggcagttgccagttgccacaaggggagcctggacctctgcatcctcctcatgatagctctgtctctctcacacacacacagtcacacagagacacgcaaatgacttctgtctctaaggctatccgcagcggttccctctaaatttttccccctatatcacttttttgggtcacatcatcaatagtttatcccctactttttcatctcccgcagcggttccccctaaatactccccctatactccactacaatataaaatattattttctatacctactttttacctactatcaatttttctactactattaattcaaaccgggcccacacgaacagtgcaagggggagagagagcgcgcgctacagccagcggagtgctgttcatctccgccgcgtggggggcctcgcagggggccgcgctgcgggcgcaggggcgcccctgtagccgccactcaaggggagggggcggtcgcgcggcgagcgctgcggccagcctaactcttccaaatttcgaagcggccaatgcaagagccagcccccggccgtatgtcaacttcacttgtctctctccaaaagatatcgtatcacccatgggcaatggccatgacccccctcccagccccaacctatatcacctagcgcagctacgctctcttctcccgctctcgctctctgcatgctagctactttctagctatctagcctctaggtccaatgcactccctccttataaacaaggaaccctccttcgcctctcttgccatagaccggacaccggagagctaggtcacaggagcgctcaggaaggccgctgagatagaggcatggcggccaatgcgggcggcggtggagcgggaggaggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggtacgtgcgtcagtgcgtgtggtgtgtgggtagtatatatggtctctccttgcattggcacgccaatcggccatcgatccaatcatatcatcgtccaaacgtatatagtacatgtgactgcaaactgatgtgcaccgtcgtcatcactgatcaggcgcggcgcctccctcgcccaccctcggcttctacgccgccggcaatggcggcggatcggctgtgctgctggacacgagttccgactggggcagcagcggcgctgccatggccaccgagacatgcttcctgcaggtcggtgctgtagtacgttcttttcttgggcattgcgcgcagtttcacgttcgtacgtacgagttgatcgccgcgtcgttccatccaccggtatatataactgttaggtacggcggtgcgcgcccgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcacttatgtctctggaatggaaggaggaggaagtgagcatagcgttggtgcgttgctgtcattgtcctaggttagtagctagtgccagttactagtaagcatcaggcataggagtatgtagtagaagcatgcacgttgccggccagccaggctttagacgggaaaagaatttggtgcagccggctgcaaaacaggatgtttacagcccccacacaaaaaaaaaagattgaccctacctgtaacaataataacacaactaaaatgttatttgatggacctacaagtgggataaatccttctttttgtgaggtgctgcaaacattctggttcatcaatttttttcctttagactccagctactgcatgcatgcatgcggtggtcctcatgtatgtcggcgtgtgtgtgtggtagcagcagcagtgaattctctagtgcagtggtggtggttgtaacgtgtagtttgggggcaggggcacagttgtgtcgaacttgtgcggttgtcccttccttttgtgacttccagaaaaaggaatgagacctgcatgtgtgtctgcttttccatgctcacctcgcctgttgtctgttgagacttcactcagccctgctggtgccttgtagttccagttccagagcatatgcaggtgcaggaatggcatctctaccactgctttcgacttgttggcttgttttcttctatgatactagtgtacttgg

SEQ ID NO: 27 - DNA, 909 bp, Zea mays; identical to SEQ ID NO: 23
  source 1..909: mol_type = genomic DNA

SEQ ID NO: 28 - DNA, 879 bp, Arabidopsis thaliana
  source 1..879: mol_type = genomic DNA
atggagcctcctcagcaccaacatcaccaccatcaggccgatcaagagtccgggaacaacaacaacaacaagtccggttccggagggtacacttgcagacagacctccaccagatggacacctaccactgagcagatcaagatcctgaaggagttgtactacaacaatgccatccgctcccctactgctgatcagattcagaagatcaccgctcgcctgcgtcaattcggtaagatcgagggtaagaacgtcttctactggttccagaaccacaaggctcgtgagaggcagaagaagcgtttcaacgggactaacatgaccactccttcctcatcacccaattctgttatgatggccgccaacgaccactaccatcctttgcttcatcaccaccacggagttcctatgcaaagacccgcaaactccgtcaacgtgaagttgaaccaggaccatcacctctaccaccacaacaagccctacccctctttcaacaacgggaatctcaaccatgcctcctctggaaccgaatgtggagttgtcaacgcctccaacggctacatgtcttcccacgtgtacggctctatggagcaagattgctccatgaactacaacaacgttggaggcggatgggcaaacatggatcaccactactcctccgctccctacaacttcttcgatagggctaaacctctcttcggcttggagggacaccaagaagaagaggaatgcggaggtgatgcttacttggaacacagaaggactctccctctctttccaatgcacggtgaggaccacatcaacggaggatctggcgctatctggaagtatggacagtccgaggtcagaccatgcgcttccctggaattgagactgaactag

SEQ ID NO: 29 - DNA, 876 bp, Lotus japonicus
  source 1..876: mol_type = genomic DNA
atggaacaaccacctcaacaacaacaacagaatgaggatggtggtgggagtagtggtggaaaaggggggtttctgagcaggcaaagcagtacacggtggactcccaccacagaccagataagaatattgaaggagctttactacaacaatggaataagatccccgagtgcagagcagattcagaggatctctgctcggctgagacagtacggcaagattgaaggcaagaatgtcttttactggttccagaaccacaaagctagagagaggcagaagaaaaggttcacttctgaaactcatcttcttcccaatcccatacaacaacaacaaagaaatggaactaatgctgcttggaagcaacctgaagaacaacccattattaaccacaccaagtattctagtaacaacatttcagctcctgcagggatcatcacttctgctccatcttcttctgcagagattgtttctgttggccagataggaaactttggttatggatctttgcctatggaaaagagttttagggactgctcaatatctgctggaggcaacacaggctatgctggaagtgctataaaccacaacctgggatggatgggtgtggatccatattcctcagcctacaccaatttctttgacaaaataaggccaactgaagaagaaaccatggaagaagaaggacaagagaatggttcaccagagattgaaacccttcctttattcccgatgcatggtgaggacattcatagtggctatttcaacctcaagtctaattcttctcactatgctggtggctggtaccagactgaagaagggtacttcaacaatggttctcgtgcttccttggagctcagcctcaaatatcctgattttgct

SEQ ID NO: 30 - DNA, 888 bp, Glycine max
  source 1..888: mol_type = genomic DNA
atggaacctcaacaacaacaacaacaagcacaagggagccaacaacaacaacaaaacgaggatggtggcagtggaaaaggggggtttctgagcaggcaaagtagtacacggtggactccaacaaacgaccagataagaatattgaaggaactttactacaacaatggaattagatccccgagtgcagagcagattcagaggatctctgctaggctgaggcagtacggtaagattgaaggcaagaatgtcttttattggttccagaaccacaaagctcgagaaaggcagaagaaaaggttcacttctgatcataatcataataatgtccccatgcaaagacccccaactaatccttctgctgcttggaaacctgatctagctgatcccattcacaccaccaagtattgtaacatctcttctactgcagggatctcttcggcatcatcttctgttgagatggttactgtgggacagatggggaattatgggtatggttctgtgcctatggagaaaagttttagggactgctcgatatcagctgggggtagcagtggccatgttggattaataaaccacaacttggggtgggttggtgtggacccatataattcctcaacctatgccaacttctttgacaaaataaggccaagtgatcaagaaacccttgaagaagaagcagagaacattggtgctactaagattgaaaccctccctttattccctatgcacggtgaggacatccacggctattgcaacctcaagtctaattcgtataactatgatggaaacggctggtatcatactgaagaagggttcaagaatgcttctcgtgcttccttggagctcagtctcaactcctacactcgcaggtctccagattatgcttaa

SEQ ID NO: 31 - DNA, 897 bp, Camelina sativa
  source 1..897: mol_type = genomic DNA
atggagccaccgcagcatcagcatcatcaccatcaagccgaccaagaaagcggcaataacaacaagtccggttctggtggttacacttgtcgccaaaccagcacgaggtggacaccaacgacggagcagatcaaaatgctcaaagagctttattacaatagtggaatccggtcaccaacagcagatcagatccagaagatcactgctaggctgcgacagttcggaaagatcgagggcaagaacgtcttttactggttccagaaccacaaggctcgtgagcgtcagaagaagagattcaacggcacaaccatgaccacaccgtcttcatctcccaactcggttatgatggctaatgatcattatcatcaacatctccctctacttcaccatcatcatggcgttcccatgcagagacctgcgaactccgtcaacgttaaacttaaccaagagcatcatctctatcatcagaacaagtcatatcccagcttcaataacgggaatttaaatcatgcaagctcaggtactgaatgtggtgctgttaatgcttctaatggctactcaagtagccatatctatggatctatggaacaagactgttctatgaattacaacaacgtaggtggaggatggacggcaaacatggatcataatcatcattactcatcagcaccttacaacttcttcgatagaccaaaacctccgtttggtctagacggtcatcaggaagaagaagaatgtggtggcgatgcttatctggaacatcgacgtacgcttcctctcttccctatgcatggtgaagatcacataaatggtgccagctggaagtatggccaactggacggtcatgattgtcatggtagaggcccttgcgcttctcttgagcttcgtttgaactag

SEQ ID NO: 32 - DNA, 897 bp, Capsella rubella
  source 1..897: mol_type = genomic DNA
atggagccaccacagcatcagcatcatcatcatcaagccgaccaagaaagcggcaacaacaacaacaagtccggctctggtggctacacgtgtcgccaaaccagcactaggtggacaccaacgacggagcagatcagaatcctcaaggatctttactacaacagtgcgatccggtcaccaacagccgatcagatccagaagatcaccgcgaggctgagacagtacgggaagatcgaaggcaagaacgtcttttactggttccagaaccataaggctcgtgagcgtcagaagaagagattcaacggcacaaccatgaccactccatcttcatctcccaactcggttatggtggctaatgatcactatcatcctctacttcaccatcatcatggtgttcccatgcaaaggcctgctaactccgtcaacgtcaaacttaaccaagaccatcatctctatcatcagaacaagtcatatcccaccttcaacaatgggaatttaaatcatggaagctcaggtactgaatgtggtgttgttaatgcttctaatggctacatgagtagccatgtctatggatctatggaacaagactgttcaatgaactacaacaacgtaggtactggaggatacggatcaaacatggatcatagtcatcattactcatctgcaccatacaacttctttgatagatcaaagcctctctttggtctagaaggtcatcaagaagatgaagaagaatatggtataggcgatgcttatctggaacatcgacgtacgcttcctctcttccctatgcatggtgaagatcacatgaacggtagtggtgctagctggaagtttgatggccgtgattgccatggtagaggcccttgcgcttctcttgagcttcgtctgaactag

SEQ ID NO: 33 - DNA, 588 bp, Arabis alpina
  source 1..588: mol_type = genomic DNA
atggctactcatgtagccattagaagcattaacagcaccacattcagaacctgaacttgcatgatttaaattcccgttattgaagctgggatatgatttgttctgatgaagcagatggtgttcttggtcatgaagcttaacgttgagagaagcaggtctctgcatggaaacaccatgatgatgttgaagaagaggatgatgatgatggttatgatgataacgatcactggccatcataactgagttgggcgatgaagacgttggtgtggtggtcatggttgtgccattgaatctcttcttctgacgctcacgagccttatggttctgaaaccagtaaaagacgtttttgccctcgatcttaccatattgtctcagccttgcagagatcttctggatctcatcagcagttggtgaccggattccactattgtagtaaagatctttgaggattctgatttgctccgtggttggagtccatctcgtgctggtttgacgacaagtgtaaccaccagaaccggacttgttgttgttgttgccgctttcttggtcggcttgatgatgatgatgatgatgttgcggtggctccat

SEQ ID NO: 34 - DNA, 873 bp, Raphanus sativus
  source 1..873: mol_type = genomic DNA
atggagccaccgcaacattaccatcaagccgatcaagaaagcggcaacaacaacaagtccgtctctggtggttacacgtgtcgtcaaacgagcacaagatggacaccaacaaccgatcaaatcagaatactcaaagatctttactacaacaatggagtccggtcaccaacagccgaccagatccagaagatctctgcaacgctgagacagtacggaaaaatcgagggaaaaaatgtcttttactggtttcaaaaccataaggctcgtgagcgacagaagaagagattcaacagcacaaccatggccacaccaacgtcttcatcgcccaactcggttatgatgatggctagtgatcaccatcatcatcatggtgttaccatccagagacctgctttggtcaacgttaagctcgaccaagaaaatcatatgtttcaccagaacagatcatatcccagctccaataacgggagtataaatcatgcaagttcaggcacggaatatggtgttttcggcgcttctaatggctacattagtaaccatatctatggatctatggaacaagactgttcaatgagctacaacagcgtaggtggaggatggacaaacatggatcataatcatcattactcaactccagcttacaacttctttgatagaccatcgcctctgtctggactagaaggtcatcaagaagaaggacaatatggtggcgattcttatctggaacatcgacgtacacttcctctcttccctttgcacggtgaggatcacatcaacggtggtggtggttccatcttgaagtacggacaatcagacggttgtgatcgttatggtagaggcccttgtgcttctcttaagctatgtctgaactga

SEQ ID NO: 35 - DNA, 885 bp, Brassica napus
  source 1..885: mol_type = genomic DNA
atggagccagcgcaacatcaccatcaagccgaccaagaaagcggcaacaacaacaagtccgtctctggtggttacacgtgtcgtcaaacgagtacaagatggacaccaacgaccgatcaaatcagaatactcaaagatctttactacaacaacggagtccggtcaccaacagccgaccagatccagaagatctctgcaaggctgagacagtacggaaagatcgagggaaaaaatgtcttttactggtttcaaaaccataaggctcgtgagcgacagaagaagagattcaacagcacaaccatgaccataccaacgtcttcatcgcccaactcggttatgatggctagtgatcactatcaccataaccatcatcatcatggcgttaccatccagagacctgctttggtcaacgttaagctcgaccaagaaaatcatatgtttcatcagaacagatcatatcccagcttcaataacgggaatacaaatcatgcaagttcaggcacggaatatggtgttttcagtgcttctaatggctacattagtagccatatctatgaacctatggaacaagactgttcaatgagctacaacaacgtaggtggaggatggacaaacatagatcataatcaccattactcaactccagcctacaacttcttagatagaccaatgcctctgtctggactagaaggtcatcatcaagaagaaggagaatatggtggcgatgcttatctggaacatagacgcacacttcctctcttccctttgcacggtgaggatcacatcaacggcggtggtggttccatctggaagtacagacaatcggacggttgtgatcgttatggtagaggcccttgtgcttctcttaagctgtgtctgaactga

SEQ ID NO: 36 - DNA, 885 bp, Brassica oleracea
  source 1..885: mol_type = genomic DNA
atggagccagcgcaacatcaccatcaagccgaccaagaaagcggcaacaacaacaagtccgtctctggtggttacacgtgtcgtcaaacgagtacaagatggacaccaacgaccgatcaaatcagaatactcaaagatctttactacaacaacggagtccggtcaccaacagccgaccagatccagaagatctctgcaaggctgagacagtacggaaagatcgagggaaaaaatgtcttttactggtttcaaaaccataaggctcgtgagcgacagaagaagagattcaacagcacaaccatgaccataccaacgtcttcatcgcccaactcggttatgatggctagtgatcactatcaccataaccatcatcatcatggcgttaccatccagagacctgctttggtcaacgttaagctcgaccaagaaaatcatatgtttcatcagaacagatcatatcccagcttcaataacgggaatataaatcatgcaagttcaggcacggaatatggtgttttcagtgcttctaatggctacattagtagccatatctatggacctatggaacaagactgttcaatgagctacaacaacgtaggtggaggatggacaaatatagatcataatcaccattactcaactccagcctacaacttcttagatagaccaatgcctctgtctggactagaaggtcatcatcaagaagaaggagaatatggtggcgatgcttatctggaacatagacgcacacttcctctcttccctttgcacggtgaggatcacatcaacggcggtggtggttccatctggaagtacagacaatcggacggttgtgatcgttatggtagaggcccttgtgcttctcttaagctgtgtctgaactga

SEQ ID NO: 37 - DNA, 882 bp, Helianthus annuus
  source 1..882: mol_type = genomic DNA
atggaaactcaacaacaacaagcagatattcaagattttgggaacaaaaacagtaacacctatgcatgcaggcaaagcagtactaggtggacacccacaagtgatcagatcagaatactgaaagagctctactacaacaatggaatcagatcaccaactgctgatcagattcagagaatcgctgctcaactcagacagtacggcaagattgaaggcaagaacgtgttttattggtttcagaaccataaagctcgtgaacgccagaagaaacggttcactcctactgctcctccaccaccgtctcacttctccgatcatattaaccaccatcttcccaacgctgcccccatgcagattccgtctcaccaccaccactaccaccatcaagaaccaccacatgtctatgctcaccctcacaaactctacaccactcatcacattggggttggttcttcttcacaaggagtgatgggtgtagggtgtggctatggatctgttgctatggagaagagtttcaggaagtgttcaatatcaccaccgggagagagtaaggccacaggaggaattggccggaatatcggttcaagatcacggataagtgtcgattcgtgttccttcttcgacacaattaagccaaagacatatgagatgtttgagaatcatgatcaagatgaagaacaaggagagccttcaactgagatcgaaacacttccattattcccgattcacgacggcaaccatcatgatttcttcggcatgaggacggcaaatctgcaattggagcaaggcactggaggttactacactggaggaagctggtatcggtctgatgagagggcgtctcttgagctcagtctcaactcatatggttattataactaa

SEQ ID NO: 38 - DNA, 795 bp, Populus trichocarpa
  source 1..795: mol_type = genomic DNA
atggaacctcaccaacaacaaccaaatgaggataataacggtggcgccaaagggaactttctttgcaggcaaaccagtacaaggtggaatcccactactgaccagataagaatattgaaggaactgtactacatcaaaggtgttaggtccccaaatggagctgagattcagcagatctctgctaggcttagaaagtacggcaagattgaaggcaagaatgtattttattggtttcagaaccataaagctcgtgagaggcaaaagaaaagactcaccaatgaagtccccatgcaacaaagaactgcctggaaacctgaagattattactcttacaagtactccaacagtaataataatcctgggttttcttcggcttcttcatctgcaaataccggtgtggttactgttgggcagacagatagccatggatatggatcggtaaccatgcaggagaagaattcttgggactgttcagcaccagctggtggtagcaatggtgctggtagtggatccatgagcaatattaactatggatcaggggttgacattaactctcacagttcatcctatgctgtatttggtcaagaacaagaagcagctgcaaagattgagactcttcctctatttccaatgcttggtgaggacatcagtagctctttcaacatcaacaatatcaacccagacttttattacagtagcggctgtggctatggtgattatggcaatgacacttcttcccgtacttccctggaccttagtctctactcctacaatggccaaccacaagattattaa

SEQ ID NO: 39 - DNA, 843 bp, Vitus vinifera
  source 1..843: mol_type = genomic DNA
atggaacctcaacaacagctccagcagcagcagcaacaaaaccaacaacagccaaacgaggatagcggcagcagcaaaggcagttttctgtgcaggcaaagcagtacacgctggactcctacaactgaccagataagaatattgaaggacctttactacaacaatggagttaggtccccaagtgctgaacagattcagaggatctcagctaggctgaggcagtacggcaagatcgaaggcaagaacgtcttttattggtttcagaaccataaagctcgtgaaaggcagaagaagcgattcactactgatatgcccatgcaaagatcccttggaaatgccggttggagacctgatgatcccattcacaacaaatttcataccattcccactcctgggatatcttccccatcttcttcttcttcacccagtgtgcttgctgttgggcagatgggaagctttggatatggatctgtggagaggagttttacagactgttcaatatcagcaggtggtggtcgtggtggggttggtggatctataaaccagagcttcgaatgggttggcatggacccatattcttcatcttatgcactctttgacaagagaaaaacaatgggtgaaagcttcgaagaagagcaagaagaagaagcaactccggagattgaaacgctcccactctttcccatgcacgctgaggatatcactggcttttgcaacattaagcccgaatccgacgcctactactccggctggtacaggcccgccgacgccaagaccagttcccgcacttctcttgagcttagcctcaactcctacgccggcaggtccccggattccccctga

SEQ ID NO: 40 - DNA, 879 bp, Arabidopsis thaliana; identical to SEQ ID NO: 28
  source 1..879: mol_type = genomic DNA

SEQ ID NO: 41 - DNA, 879 bp, Lotus japonicus
  source 1..879: mol_type = genomic DNA
atggaacaacctccccaacagcagcaacaaaacgaagacggaggcggatcaagtgggggaaaaggagggttcctcagtaggcagtccagtactaggtggacacccaccacagaccagattcgcatcctcaaggagctctactacaacaacggaatacgctcaccctccgcagaacagatccagaggatctccgcaagactcagacagtacggcaagatcgagggcaagaacgtcttctactggttccaaaaccacaaagcccgcgaaaggcagaagaagcgcttcaccagtgagacacacttgctgcccaacccaattcagcagcaacagcgcaacggaacaaacgcagcttggaagcagccagaggagcaacccatcatcaaccacaccaagtactcctccaacaacatatccgcacccgcagggattatcacaagtgccccatcttcttccgcagaaatagtctccgtggggcagatcggaaacttcggatacggatctctgccaatggagaagtccttcagggactgcagtatctcagccggaggtaacacaggatacgccgggtctgctattaaccacaacctcggatggatgggagtcgacccatactcctctgcttacaccaacttcttcgacaaaatacgcccaacagaggaggagactatggaggaagaaggacaggagaacggctcaccagaaatcgagaccctgcctttgttcccaatgcacggagaggacatccactccggctacttcaacctcaagagtaactcatcccactacgctggcggatggtaccaaactgaggaaggctacttcaacaacggatcacgcgccagtctggagctgtccctgaagtaccctgacttcgcttag

SEQ ID NO: 42 - DNA, 909 bp, Medicago trunculata
  source 1..909: mol_type = genomic DNA
atggaacaacctcagcagcagcagcaaccacagacccaacagcactctccaaacaacggaatcatggggtccagacagagttccaccagatggaccccaacaaccgaccagatccgcattctcaaagacctctactacaacaacggaataaggtctccaagtgccgagcaaatccagaggatctccgcaaggctcagacagtacgggaagatcgagggcaagaacgtcttctactggttccaaaaccacaaagccagagagcgccagaaaaagcgcttcacctccgacgtcaacgtcgtccctatcatccagcgcgcccccaacaacaatactatcataagtgccgccaactggaagcctgaccaccacgaacaacagcagaacattaatgtccacacaaaccactccacatacaacatctcctccgctggcctcagttctgcttcctgctcctcagcagaaatggtcactgtggggcaaataggcaactacggctacgggtctgtcccaatggagaagtctttccgcgaatgcaccatctcagctggatgctcatcatcccaggttggctcaaccataaacccccacatcggctggatcggacaccacgtcgacccatattcaagtgcctacgctaacctcttcgagaaaatacgccccaacgaggagatcatggaagagtacgaccagggacaggaaaacggttccccagagatcgagacactcccactcttccctatgcacggagaagacatccacggagggtattgcaacctcaaatcaaattcctccaactacggaggatggtaccaggcagaagacgcagggttcatgtacggaagtcgtaccacttctctcgagctcagtctgaactcctacggatgcaggtcccccgactacgccaattaa

SEQ ID NO: 43 - DNA, 924 bp, Petunia hybrida
  source 1..924: mol_type = genomic DNA
atggaaacagcacagcaccagcagaacaaccaacagcactacctccaccaacacctctccatcggacaaggaacaaacatcgaggacggcagtaacaagaacaactcctccaacttcatgtgcaggcaaaatagtacccgctggacaccaaccactgaccagatcaggatattaaaggacctctactacaacaacggagtcagatcacctaccgctgaacagatccagagaatctccgccaagctcaggcagtacggcaagatcgagggcaagaacgtcttctactggttccagaaccataaagcccgcgagagacaaaagaagcgcctcatcgccgcagccaccaccgataacactaacctcccaatgcagatgcagttccagagaggagtctggagatcctctgcagacgaccccatccaccacaagtatactaatccaggagtgcactgcccttcagcttcttcccacggagttctcgcagtcggacagaacggcaatcacggatacggagccttggcaatggaaaaatccttccgcgactgctccatcagtccaggcagttccatgtcacaccaccaccaccaaaacttcgcttgggcaggagtcgatccttattcctcaaccacaacctaccctttcctcgagaagaccaagcacttcgagaatgaaaccctcgaagctgatgaagagcagcaagaggaggaccaggagaactactactaccaaaggaccacctctgccatcgaaaccctgcctctcttccctatgcacgaggagaacatctcctccttctgcaacctcaagcaccaggagtcatccggaggattctacaccgagtggtacagagcagacgacaacctcgcagcagccagagcttccctcgagctctccttgaactccttcatcggcaactcctcctag

SEQ ID NO: 44 - DNA, 864 bp, Phaseolus vulgaris
  source 1..864: mol_type = genomic DNA
atggaaccacagcagccccagggcagtcaaccaaacgaggacggtggaagtgggaaaggcggatttctctccagacagtcctccactaggtggactccaaccactgaccagatcagaatactcaaagacctctactacaacaacggaatcagatccccttccgcagaacagatccaaaggatctccgctaggctcagacagtacgggaagatcgagggcaagaacgtcttctactggttccaaaaccacaaggccagggagagacagaagaagaggttcacctccgacaacgtccccatgcaaagacctgcaccaacaaatgctgcacctccttggaaacccgaccaagaccccattcacaccaagtactccaacatctcctccacaggaatctccagtgcatcctcctcctcagtcgagatgatcaccgtcggacaaatggggaactacgggtacggaagtgtgccaatggaaaagtccttcagagactgctccatatccgccggaggatcttccggtcatgttggaataaaccacaacctcggctgggtgggagtcgatccttactcttctgcatacgccaacttcttcgataaaataagaccaaacgaagagaccctcgaggaagaagaagaggaggaggaggaggaagaagaagaggacggaggagcagaaattgagaccctccccctgttcccaatgcacggagaggacattcacggctactgcaacctcaagtccaacagttacaactacgacggcaacggctggtatcacagtgaggatggcttcaaaaacggcagtagggcctccctcgagctctccctcaactcttacaccagaagatcccccgacttcgcatag

SEQ ID NO: 45 - DNA, 963 bp, Zea mays
  source 1..963: mol_type = genomic DNA
atggcggccaacgtgggcgcgggcaggagtgctggcggcggcggagccggcactggcactggcactgctgctggcagcggcggcgtgtcgacggccgtgtgccgccctagcggctcgcggtggacgccgacgccggagcagatcaggatcctcaaggagctctactacggctgcggcatccggtcgcccaactcggagcagatccagcgcatcaccgccatgctgcggcagcacggcaagatcgagggcaagaacgtcttctactggttccagaaccacaaggcccgcgagcgccagaagcgccgcctcaccaacctcgacgtcaacgtgcccgtcgccgccgacgacagcgcccaccgccttggcgtcctctcgttgtcgccttcttcaggttgttcaggcgcggcgcctccgtcgcccaccctcggcttctacgccggcggcaatggctccgctgtgatgctggacacgagttccgattggggcagcgctgctgccatggccactgaggcatgcttcatgcaggactacatgggcgtgatgggcggcgcgtcaccgtgggcatgctcctcctcgtcgtcggaggacccgatggcggcgctggcgctggcgccgaaggtgacccgggcgcccgagacgctccctctcttcccgaccggcggcggcggagacgataggcagcccccgcggccgcggcagtctgtcccagcaggcgaggccatccgcggcggcagcagcagcagcagctaccttccgttctggggtgccgcgcccaccccaactggcagtgccacttccgttgcgatccagcagcaacaccagctgatgcagatgcaagagcagtacagcttttacagcaacgcccagctgctgcccggcaccggcagccaggatgcagcagcaacatccctggagctgagcctcagctcctggtgctccccttaccctgcagggaccatgtga

SEQ ID NO: 46 - DNA, 978 bp, Zea mays
  source 1..978: mol_type = genomic DNA
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggcgcggcgcctccctcgcccaccctcggcttctacgccgccggcaatggcggcggatcggctgggctgctggacacgagttccgactggggcagcagcggcgctgccatggccaccgagacatgcttcctgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtga

SEQ ID NO: 47 - DNA, 666 bp, Zea mays
  source 1..666: mol_type = genomic DNA
atggaggcgctgagcgggcgggtaggcgtcaagtgcgggcggtggaaccctacggcggagcaggtgaaggtcctgacggagctcttccgcgcggggctgcggacgcccagcacggagcagatccagcgcatctccacccacctcagcgccttcggcaaggtggagagcaagaacgtcttctactggttccagaaccacaaggcccgcgagcgccaccaccacaagaagcgacgccgcggcgcgtcgtcgtcctcccccgacagcggcagcggcaggggaagcaacaacgaggaagacggccgtggtgccgcctcgcagtcgcacgacgccgacgccgacgccgacctcgtgctgcaaccgccagagagcaagcgggaggccagaagctatggccaccatcaccggctcgtgacatgctacgtcagggacgtggtggagcagcaggaggcgtcgccgtcgtgggagcggccgacgagggaggtggagacgctagagctcttccccctcaagtcgtacggcgacctcgaggcggcggagaaggtccggtcgtacgtcagaggaagcggcgccaccagcgagcagtgcagggagttgtccttcttcgacgtcgtctccgccggccgggatccgccgctcgagctcaggctctgcagcttcggtccctag

SEQ ID NO: 48 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 49 - protein, 343 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPSGAAPPSPTLGFYAAGNGGGSAVLLDTSSDWGSSGAAMATETCFLQVGAVVRSFLGHCAQFHVRTYELIAASFHPPVYITVRYGGARPQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 50 - protein, 325 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAGLLDTSSDWGSSGAAMATETCFLQDYMGVTDTGSSSQWPCFSSSDTIMAAAAAAARVATTRAPETLPLFPTCGDDDDDDSQPPPRPRHAVPVPAGETIRGGGGSSSSYLPFWGAGAASTTAGATSSVAIQQQHQLQEQYSFYSNSTQLAGTGSQDVSASAAALELSLSSWCSPYPAAGSM

SEQ ID NO: 51 - protein, 300 aa, Zea mays
MAANAGGGGAGGGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 52 - protein, 302 aa, Zea mays; identical to SEQ ID NO: 48

SEQ ID NO: 53 - protein, 292 aa, Arabidopsis thaliana
MEPPQHQHHHHQADQESGNNNNNKSGSGGYTCRQTSTRWTPTTEQIKILKELYYNNAIRSPTADQIQKITARLRQFGKIEGKNVFYWFQNHKARERQKKRFNGTNMTTPSSSPNSVMMAANDHYHPLLHHHHGVPMQRPANSVNVKLNQDHHLYHHNKPYPSFNNGNLNHASSGTECGVVNASNGYMSSHVYGSMEQDCSMNYNNVGGGWANMDHHYSSAPYNFFDRAKPLFGLEGHQEEEECGGDAYLEHRRTLPLFPMHGEDHINGGSGAIWKYGQSEVRPCASLELRLN

SEQ ID NO: 54 - protein, 292 aa, Lotus japonicus
MEQPPQQQQQNEDGGGSSGGKGGFLSRQSSTRWTPTTDQIRILKELYYNNGIRSPSAEQIQRISARLRQYGKIEGKNVFYWFQNHKARERQKKRFTSETHLLPNPIQQQQRNGTNAAWKQPEEQPIINHTKYSSNNISAPAGIITSAPSSSAEIVSVGQIGNFGYGSLPMEKSFRDCSISAGGNTGYAGSAINHNLGWMGVDPYSSAYTNFFDKIRPTEEETMEEEGQENGSPEIETLPLFPMHGEDIHSGYFNLKSNSSHYAGGWYQTEEGYFNNGSRASLELSLKYPDFA

SEQ ID NO: 55 - protein, 295 aa, Glycine max
MEPQQQQQQAQGSQQQQQNEDGGSGKGGFLSRQSSTRWTPTNDQIRILKELYYNNGIRSPSAEQIQRISARLRQYGKIEGKNVFYWFQNHKARERQKKRFTSDHNHNNVPMQRPPTNPSAAWKPDLADPIHTTKYCNISSTAGISSASSSVEMVTVGQMGNYGYGSVPMEKSFRDCSISAGGSSGHVGLINHNLGWVGVDPYNSSTYANFFDKIRPSDQETLEEEAENIGATKIETLPLFPMHGEDIHGYCNLKSNSYNYDGNGWYHTEEGFKNASRASLELSLNSYTRRSPDYA

SEQ ID NO: 56 - protein, 298 aa, Camelina sativa
MEPPQHQHHHHQADQESGNNNKSGSGGYTCRQTSTRWTPTTEQIKMLKELYYNSGIRSPTADQIQKITARLRQFGKIEGKNVFYWFQNHKARERQKKRFNGTTMTTPSSSPNSVMMANDHYHQHLPLLHHHHGVPMQRPANSVNVKLNQEHHLYHQNKSYPSFNNGNLNHASSGTECGAVNASNGYSSSHIYGSMEQDCSMNYNNVGGGWTANMDHNHHYSSAPYNFFDRPKPPFGLDGHQEEEECGGDAYLEHRRTLPLFPMHGEDHINGASWKYGQLDGHDCHGRGPCASLELRLN

SEQ ID NO: 57 - protein, 298 aa, Capsella rubella
MEPPQHQHHHHQADQESGNNNNKSGSGGYTCRQTSTRWTPTTEQIRILKDLYYNSAIRSPTADQIQKITARLRQYGKIEGKNVFYWFQNHKARERQKKRFNGTTMTTPSSSPNSVMVANDHYHPLLHHHHGVPMQRPANSVNVKLNQDHHLYHQNKSYPTFNNGNLNHGSSGTECGVVNASNGYMSSHVYGSMEQDCSMNYNNVGTGGYGSNMDHSHHYSSAPYNFFDRSKPLFGLEGHQEDEEEYGIGDAYLEHRRTLPLFPMHGEDHMNGSGASWKFDGRDCHGRGPCASLELRLN

SEQ ID NO: 58 - protein, 304 aa, Arabis alpina
MEPPQHHHHHHQADQESGNNNNKSGSGGYTCRQTSTRWTPTTEQIRILKDLYYNSGIRSPTADEIQKISARLRQYGKIEGKNVFYWFQNHKARERQKKRFNGTTMTTTPTSSSPNSVMMASDRYHHNHHHHPLLQHHHGVSMQRPASLNVKLHDQEHHLLHQNKSYPSFNNGNLNHASSGSECGAVNASNGYMSSHVYGSMEQDYSMSNNNIGGGGWSNMDHNHHYSSPAYNFFDRPKPLFGLEGHQEEDQYGGDAYLEHRRTLPLFPMHGEDHINGGGGAIWKYRQLDGLELRLNSYAGVAPE

SEQ ID NO: 59 - protein, 290 aa, Raphanus sativus
MEPPQHYHQADQESGNNNKSVSGGYTCRQTSTRWTPTTDQIRILKDLYYNNGVRSPTADQIQKISATLRQYGKIEGKNVFYWFQNHKARERQKKRFNSTTMATPTSSSPNSVMMMASDHHHHHGVTIQRPALVNVKLDQENHMFHQNRSYPSSNNGSINHASSGTEYGVFGASNGYISNHIYGSMEQDCSMSYNSVGGGWTNMDHNHHYSTPAYNFFDRPSPLSGLEGHQEEGQYGGDSYLEHRRTLPLFPLHGEDHINGGGGSILKYGQSDGCDRYGRGPCASLKLCLN

SEQ ID NO: 60 - protein, 294 aa, Brassica napus
MEPAQHHHQADQESGNNNKSVSGGYTCRQTSTRWTPTTDQIRILKDLYYNNGVRSPTADQIQKISARLRQYGKIEGKNVFYWFQNHKARERQKKRFNSTTMTIPTSSSPNSVMMASDHYHHNHHHHGVTIQRPALVNVKLDQENHMFHQNRSYPSFNNGNTNHASSGTEYGVFSASNGYISSHIYEPMEQDCSMSYNNVGGGWTNIDHNHHYSTPAYNFLDRPMPLSGLEGHHQEEGEYGGDAYLEHRRTLPLFPLHGEDHINGGGGSIWKYRQSDGCDRYGRGPCASLKLCLN

SEQ ID NO: 61 - protein, 294 aa, Brassica oleracea
MEPAQHHHQADQESGNNNKSVSGGYTCRQTSTRWTPTTDQIRILKDLYYNNGVRSPTADQIQKISARLRQYGKIEGKNVFYWFQNHKARERQKKRFNSTTMTIPTSSSPNSVMMASDHYHHNHHHHGVTIQRPALVNVKLDQENHMFHQNRSYPSFNNGNINHASSGTEYGVFSASNGYISSHIYGPMEQDCSMSYNNVGGGWTNIDHNHHYSTPAYNFLDRPMPLSGLEGHHQEEGEYGGDAYLEHRRTLPLFPLHGEDHINGGGGSIWKYRQSDGCDRYGRGPCASLKLCLN

SEQ ID NO: 62 - protein, 298 aa, Helianthus annuus
METQQQQADIQDFGNKNSNTYACRQSSTRWTPTSDQIRILKELYYNNGIRSPTADQIQRIAAQLRQYGKIEGKNVFYWFQNHKARERQKKRFTPTAPPPPSHFSDHINHHLPNAAPMQIPSHHHHYHHQEPPHVYAHPHKLYTTHHIGTHNIRVGSSSQGVMGVGCGYGSVAMEKSFRKCSISPPGESKATGGIGRNIGSRSRISVDSCSFFDTIKPKTYEMFENRDQDEEQGEPSTEIETLPLFPIHDGNHHDFFGMRTANLPLEQGTGGYYTGGSWYRSDERASLELSLNSYGYYN

SEQ ID NO: 63 - protein, 264 aa, Populus trichocarpa
MEPHQQQPNEDNNGGAKGNFLCRQTSTRWNPTTDQIRILKELYYIKGVRSPNGAEIQQISARLRKYGKIEGKNVFYWFQNHKARERQKKRLTNEVPMQQRTAWKPEDYYSYKYSNSNNNPGFSSASSSANTGVVTVGQTDSHGYGSVTMQEKNSWDCSAPAGGSNGAGSGSMSNINYGSGVDINSHSSSYAVFGQEQEAAAKIETLPLFPMLGEDISSSFNINNINPDFYYSSGCGYGDYGNDTSSRTSLDLSLYSYNGQPQDY

SEQ ID NO: 64 - protein, 280 aa, Vitus vinifera
MEPQQQLQQQQQQNQQQPNEDSGSSKGSFLCRQSSTRWTPTTDQIRILKDLYYNNGVRSPSAEQIQRISARLRQYGKIEGKNVFYWFQNHKARERQKKRFTTDMPMQRSLGNAGWRPDDPIHNKFHTIPTPGISSPSSSSSPSVLAVGQMGSFGYGSVERSFTDCSISAGGGRGGVGGSINQSFEWVGMDPYSSSYALFDKRKTMGESFEEEQEEEATPEIETLPLFPMHAEDITGFCNIKPESDAYYSGWYRPADAKTSSRTSLELSLNSYAGRSPDSP

SEQ ID NO: 65 - protein, 292 aa, Arabidopsis thaliana; identical to SEQ ID NO: 53

SEQ ID NO: 66 - protein, 292 aa, Lotus japonicus; identical to SEQ ID NO: 54

SEQ ID NO: 67 - protein, 302 aa, Medicago trunculata
MEQPQQQQQPQTQQHSPNNGIMGSRQSSTRWTPTTDQIRILKDLYYNNGIRSPSAEQIQRISARLRQYGKIEGKNVFYWFQNHKARERQKKRFTSDVNVVPIIQRAPNNNTIISAANWKPDHHEQQQNINVHTNHSTYNISSAGLSSASCSSAEMVTVGQIGNYGYGSVPMEKSFRECTISAGCSSSQVGSTINPHIGWIGHHVDPYSSAYANLFEKIRPNEEIMEEYDQGQENGSPEIETLPLFPMHGEDIHGGYCNLKSNSSNYGGWYQAEDAGFMYGSRTTSLELSLNSYGCRSPDYAN

SEQ ID NO: 68 - protein, 307 aa, Petunia hybrida
METAQHQQNNQQHYLHQHLSIGQGTNIEDGSNKNNSSNFMCRQNSTRWTPTTDQIRILKDLYYNNGVRSPTAEQIQRISAKLRQYGKIEGKNVFYWFQNHKARERQKKRLIAAATTDNTNLPMQMQFQRGVWRSSADDPIHHKYTNPGVHCPSASSHGVLAVGQNGNHGYGALAMEKSFRDCSISPGSSMSHHHHQNFAWAGVDPYSSTTTYPFLEKTKHFENETLEADEEQQEEDQENYYYQRTTSAIETLPLFPMHEENISSFCNLKHQESSGGFYTEWYRADDNLAAARASLELSLNSFIGNSS

SEQ ID NO: 69 - protein, 287 aa, Phaseolus vulgaris
MEPQQPQGSQPNEDGGSGKGGFLSRQSSTRWTPTTDQIRILKDLYYNNGIRSPSAEQIQRISARLRQYGKIEGKNVFYWFQNHKARERQKKRFTSDNVPMQRPAPTNAAPPWKPDQDPIHTKYSNISSTGISSASSSSVEMITVGQMGNYGYGSVPMEKSFRDCSISAGGSSGHVGINHNLGWVGVDPYSSAYANFFDKIRPNEETLEEEEEEEEEEEEEDGGAEIETLPLFPMHGEDIHGYCNLKSNSYNYDGNGWYHSEDGFKNGSRASLELSLNSYTRRSPDFA

SEQ ID NO: 70 - protein, 320 aa, Zea mays
MAANVGAGRSAGGGGAGTGTGTAAGSGGVSTAVCRPSGSRWTPTPEQIRILKELYYGCGIRSPNSEQIQRITAMLRQHGKIEGKNVFYWFQNHKARERQKRRLTNLDVNVPVAADDSAHRLGVLSLSPSSGCSGAAPPSPTLGFYAGGNGSAVMLDTSSDWGSAAAMATEACFMQDYMGVMGGASPWACSSSSSEDPMAALALAPKVTRAPETLPLFPTGGGGDDRQPPRPRQSVPAGEAIRGGSSSSSYLPFWGAAPTPTGSATSVAIQQQHQLMQMQEQYSFYSNAQLLPGTGSQDAAATSLELSLSSWCSPYPAGTM

SEQ ID NO: 71 - protein, 325 aa, Zea mays; identical to SEQ ID NO: 50

SEQ ID NO: 72 - protein, 221 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGSGATSEQCRELSFFDVVSAGRDPPLELRLCSFGP

SEQ ID NO: 73 - DNA, 368 bp, Zea mays
  source 1..368: mol_type = genomic DNA
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgcc

SEQ ID NO: 74 - DNA, 971 bp, Zea mays
  source 1..971: mol_type = genomic DNA
ccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctag

SEQ ID NO: 75 - DNA, 896 bp, Zea mays
  source 1..896: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 76 - DNA, 189 bp, Solanum tuberosum
  source 1..189: mol_type = genomic DNA
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcag

SEQ ID NO: 77 - DNA, 795 bp, Escherichia coli
  source 1..795: mol_type = genomic DNA
atggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 78 - DNA, 48 bp, synthetic construct
  misc_feature 1..48: note = donor DNA homology arm 1
  source 1..48: mol_type = other DNA
gaagttcctattccgaagttcctattctctagaaagtataggaacttc

SEQ ID NO: 79 - DNA, 48 bp, synthetic construct
  misc_feature 1..48: note = donor DNA homology arm 2
  source 1..48: mol_type = other DNA
gaagttcctattccgaagttcctattcttcaaaaagtataggaacttc

SEQ ID NO: 80 - DNA, 20 bp, synthetic construct
  misc_feature 1..20: note = gRNA
  source 1..20: mol_type = other DNA
ggattccgcggaaatgggtg

SEQ ID NO: 81 - DNA, 19 bp, synthetic construct
  misc_feature 1..19: note = gRNA
  source 1..19: mol_type = other DNA
gtcaaggacatacgagacc

SEQ ID NO: 82 - DNA, 77 bp, synthetic construct
  misc_feature 1..77: note = gRNA
  source 1..77: mol_type = other DNA
gttttagagctagaaatagcaagttaaaataaggctagtccgttatcaacttgaaaaagtggcaccgagtcggtgct

SEQ ID NO: 83 - DNA, 297 bp, Arabidopsis thaliana
  source 1..297: mol_type = genomic DNA
acgtgcgccaaggacatccaaaaagcagccgcggaggcggcactcgcctttcaggacgagacctgcgacaccaccaccaccaaccacggcctcgacatggaggagaccatggtcgaagccatctacacccccgagcagagcgagggcgccttctacatggacgaagagacaatgttcggcatgccaaccctcctcgacaacatggccgaaggcatgctgctgccgcccccgagcgtccagtggaaccacaactacgacggcgaaggcgacggggatgtgtccctgtggagctactag

SEQ ID NO: 84 - DNA, 1000 bp, Zea mays
  source 1..1000: mol_type = genomic DNA
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 85 - DNA, 18 bp, synthetic construct
  misc_feature 1..18: note = gRNA
  source 1..18: mol_type = other DNA
gcctttgcagtttgcacc

SEQ ID NO: 86 - DNA, 18 bp, synthetic construct
  misc_feature 1..18: note = gRNA
  source 1..18: mol_type = other DNA
gcctttgcagtttgcacc

SEQ ID NO: 87 - DNA, 18 bp, synthetic construct
  misc_feature 1..18: note = gRNA
  source 1..18: mol_type = other DNA
gttgccacaaggggagcc

SEQ ID NO: 88 - DNA, 20 bp, synthetic construct
  misc_feature 1..20: note = gRNA
  source 1..20: mol_type = other DNA
gcaaatgacttctgtctcta

SEQ ID NO: 89 - DNA, 20 bp, synthetic construct
  misc_feature 1..20: note = gRNA
  source 1..20: mol_type = other DNA
gactcttccaaatttcgaag

SEQ ID NO: 90 - DNA, 20 bp, synthetic construct
  misc_feature 1..20: note = gRNA
  source 1..20: mol_type = other DNA
gtcgtatcacccatgggcaa

SEQ ID NO: 91 - DNA, 971 bp, Zea mays; identical to SEQ ID NO: 74
  source 1..971: mol_type = genomic DNA

SEQ ID NO: 92 - DNA, 91 bp, Zea mays
  source 1..91: mol_type = genomic DNA
ctctagcaaagcacttgccatctaccgaccgccgcattccaaacagcccgacgagctagcagagcggcaggcacctccctcctcaaggaac

SEQ ID NO: 93 - DNA, 1200 bp, Setarica italica
  source 1..1200: mol_type = genomic DNA
tagcgtggcatttggcaccgccgcacactatgcgagctaggagtgctagtttacagatgaaactatgaaaccaagaaaacacaattgtccgtagctagcgcgtggcgtcgtgtcaagcttcacacagtatcttcgttcttcactataaatcgcgcctgttgttcgtgagacccaccaccgtggataacagtcccctaataataagcgatcaacagcactgctgatggctgaacaccgaggcgccgccgcgcgtaaccatccgccggagcagcccgtcgtgccggcgaacgacgccttccccgaccgcgcccagcagctctgcctcgacgccgggcgcgcgctcgcgatgtgcggcgtgctcgtcgccgtccctcccgcattttacgcgtcgacgtcgcggacgacaccgcacttgccttcgtcggcttcgtcctctggatcatcggtgcgtggctgtgcctcctggcgctcacgcccttggctccacgggcggtgagggcgggcggggccgccgccagcaccttgctgatcaggtgcctgtccctttcatgaactactagcgcccgcgcccgcggccgccgggcacaagagaacacatcgatgttcgctgcgtacgtcagcaataaggcactctttgtcagtagtcgagtgcatatgttgggtgtttgccctgggagatttcaagtgttgtggccattatcccgcaggataaaaaatcggcggcttttggattgatacggaaaataacgatgtttcatgctcgtgttgtgtgtgtgacatcttgtcagccaagtttttaaccgttgctactccccctctgtgggctcagtgtgtggaccggaatacgtcgtacataggccgtagcctaagcccaatatgccgtttttcaggttggcctgtgtgacgacaagcgggtctcaaaagcccatcacgcaactggcctgcaattattggaggagagcgcgtcgagttggttccagaggctgcgtggtggggccggactctgcgacgacctctcgttcgcgtccgatttggttgccgtgagccgtgacgtaacgcctccttccgttcctccctatataagttccacccccgggtccggtttccccaagccccagtgacccccgatcccgatcgcgaaatctccgcacccgccggcaagggaccgaagcaagcctctctcccgaccgtcgcatcaag

SEQ ID NO: 94 - DNA, 1021 bp, Setarica italica
  source 1..1021: mol_type = genomic DNA
gtacgcgtccgctcgatctgtcctcttccttctcgatttgacttgctgtggtgcccgatcacgggatcagaatcgcaccagtgtagatttcgcatgttctaagatgtgttagtttccaggctaatccagttctcttgacgcccgatccgatcgagctccgtgtttatttcagcctaatcgtagtattcccctaaaatttcctcattagggggttcgcaagattgttggtcgacaggttcacttatgctcgatcgcttacttttagtcgattcatgctgcgtactattgttgccccccgtggttttatcttttctcgtgtgaaatccagtagatatagtagatatcgtagcattaggttttaattcaatcgattgattattgtatcatcaaaattgagaagttcgaatcatgcctatcatgctgcctcaaccgaaatcctgtatattgaagtttcaattcaattgattgatcattcattagattcaaccaaaatctagaaggttagatgacgcatgtcatgctgcctgcgtagtttacctttgatctgtttatgattaccttatcagtaggatatttgtgattttgggaagtagcactaactgattcatgctgacatgatgttccctttcagattatactgttgagtcctatctaattgatgctttgtatgctatatgaatacgtcatggtaaattagcagttaattagcatattagcaattgggcgtacctcatcaggattgctcttgtagatgtgtgtggtgatgtttcttgttcaattcatgttctgctttaaaagtatgtgttctctgtttgttaattaagattcatggttataatgatttggacctaattaatcagggttgcccttttagatgcgtggttacttgttgagttcatttttttcctgctatagttgtatgcgtttgttcattcatggttagatttgatttgtttcttctctattccatgccttccatatacttcaattgtagcttaacatgagatttctgtatatgcag

SEQ ID NO: 95 - DNA, 310 bp, Solanum tuberosum
  source 1..310: mol_type = genomic DNA
ctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 96 - DNA, 870 bp, Sorghum bicolor
  source 1..870: mol_type = genomic DNA
gcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaac

SEQ ID NO: 97 - DNA, 1917 bp, Zea mays
  source 1..1917: mol_type = genomic DNA
atggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactga

SEQ ID NO: 98 - DNA, 997 bp, Sorghum bicolor
  source 1..997: mol_type = genomic DNA
gccagatcctcggtgtacaaataacccgtcttatcctatgagacgggccggcgtcagtgtgttctggaggaatttttatgtcagagcctttttctttgtgtgcttgatgtagatgccaagggaagcttattggctgttgaagcttgatgcaaaataaattatggaactctgttttttgtttatctaataataactagcaaatatgcttccattgcattgaaactaacagccttttgtgtttccaagttttattttgtgacaatgtcatctatttcaattagttgtggaatcggaaacttgcaggactaacttggaaactccaatccctcagcatcctggactttttcctggtgtaatccatgtagatattattttaatcatcattttagttctggaggtttttccatctccggttttgctcccctttcttcaaaaaaaaaaaaaaaatgccgtaggcgccgcaacgcccacctgttgttcaaactcatgggcacgagtggctcgaagattttatacaacaattgctgtagtttcaccgttgctggtgaagaagcatttttttaaaaaaatatagtggtattcattttaattagtttagttgtgcagcgagcaaaatttggacatgctctgctcggaatctgatcgacctagacacagattagcagcagtagctttgtcatctgttccaagagttgcgatctgatagaagaaaaaaaaaccttcccttcaatgtaaaaccgaaactaacaagaaagaagcacagtgccgtttaggcaagtatggagtacgtattaagcatgtagaaggccatgcatgaaccactaacaagaaagaagcgcagtgccattcaggcaagcataagcatgtagattggcatgcatgaacaactaacagtagatcgtctctggtctgattagaagttttttgggaagccaagaaatcatgtacaactggttccatctcaaattccgtggcaaaaagaggcctaagcaacata

SEQ ID NO: 99 - DNA, 881 bp, Oryza sativa
  source 1..881: mol_type = genomic DNA
tgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcacc

SEQ ID NO: 100 - protein, 264 aa, Escherichia coli
MVEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 101 - protein, 98 aa, Arabidopsis thaliana
TCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 102 - DNA, 4293 bp, Streptococcus pyogenes
  source 1..4293: mol_type = genomic DNA
atggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagac

SEQ ID NO: 103 - DNA, 375 bp, Streptococcus pyogenes
  source 1..375: mol_type = genomic DNA
atggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgag

SEQ ID NO: 104 - DNA, 3729 bp, Streptococcus pyogenes
  source 1..3729: mol_type = genomic DNA
gtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagac

SEQ ID NO: 105 - DNA, 4293 bp, Streptococcus pyogenes
  source 1..4293: mol_type = genomic DNA
atggacaagaagtacagcatcggcctcgccatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgacgccatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagac

SEQ ID NO: 106 - protein, 360 aa, synthetic construct
  REGION 1..360: note = WUS consensus sequence
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPNSGAAPPSPTLGFYAAGNGGGSAVLLDTSSDWGSSGAAMATETCFLQDNNNNNNNNNNNNNNNNNNNNNNNNNNNNNYMGVTDTGSSSQWPRFSSSDTIMAAAAARANNATTRAPETLPLFPTCGDDGGSGSNNNNNNNNNNNNNNNNNNNNNNNNNSSYLPFWGNNAASTTAGATSSVAIQQQHQLQEQYSFYSNSNSTQLAGTGNQDVSATAAAAAALELSLSSWCSPYPAAGSM

SEQ ID NO: 107 - protein, 1368 aa, Streptococcus pyogenes [sequence omitted]

SEQ ID NO: 108 - protein, 125 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDE

SEQ ID NO: 109 - protein, 1243 aa, Streptococcus pyogenes
VAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 110 - protein, 1425 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVSFCFYLYIYNNYHLVVIYFKYFFQNKRMYIAIAFLFISVYILIYNFSNIPKHGDVQVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD